(19) United States
(12) Patent Application Publication
(10) Pub. No.: US 2020/0024574 A1
(43) Pub. Date: Jan. 23, 2020

(54) STEM CELL-DERIVED ASTROCYTES, METHODS OF MAKING AND METHODS OF USE

(71) Applicant: MEMORIAL SLOAN-KETTERING CANCER CENTER, New York, NY (US)

(72) Inventors: Lorenz Studer, New York, NY (US); Jason Tchieu, New York, NY (US)

(73) Assignee: MEMORIAL SLOAN-KETTERING CANCER CENTER, New York, NY (US)

(21) Appl. No.: 16/576,956

(22) Filed: Sep. 20, 2019

Related U.S. Application Data
(63) Continuation of application No. PCT/US2018/023551, filed on Mar. 21, 2018.
(60) Provisional application No. 62/474,596, filed on Mar. 21, 2017.
(60) Provisional application No. 62/474,429, filed on Mar. 21, 2017.

(51) Int. Cl. C12N 5/079; A61K 35/30

(52) U.S. Cl. CPC C12N 5/0622; A61K 35/30; C12N 2501/11; C12N 2501/115; C12N 2501/13; C12N 2506/45; C12N 2501/155; C12N 2501/235; C12N 2501/41; C12N 2506/02; C12N 2501/15

(57) ABSTRACT

The presently disclosed subject matter provides in vitro methods of inducing differentiation of stem cells into glial competent cells (e.g., astrocyte precursors) and astrocytes, and glial competent cells (e.g., astrocyte precursors) and astrocytes generated by such methods. The presently disclosed subject matter also provides uses of such glial competent cells (e.g., astrocyte precursors) and astrocytes for treating neurodegenerative disorders.

### CROSS-REFERENCE TO RELATED APPLICATION

This application is a Continuation of International Application No. PCT/US2018/023551 filed on Mar. 21, 2018, which claims priority to U.S. Provisional Application No. 62/474,429 filed on Mar. 21, 2017 and U.S. Provisional Application No. 62/474,596 filed on Mar. 21, 2017, the contents of each of which are incorporated by reference in their entirety, and to each of which priority is claimed.

### 1. INTRODUCTION

The presently disclosed subject matter relates to glial competent cells (e.g., astrocyte cell precursors) and astrocyte cells derived from stem cells (e.g., human stem cells) and uses thereof for cell-based treatment of neurological disorders.

### 2. BACKGROUND OF THE INVENTION

Astrocytes are glial cells that function to regulate amino acid, nutrient and ion metabolism in the brain, couple neuronal activity and cerebral blood flow, and modulate excitatory synaptic transmission. Astrocytes have been reported as having inclusion bodies in brains of patients with prion disease, Alzheimer's disease, and Parkinson's disease, and correlate with severity of disease in post mortem brains. Additionally, when Mecp2 is knocked out of astrocytes, the cells failed to support normal dendritic morphology of wild type neurons, and further, have been observed to release mitochondria that enter neurons after stroke. Accordingly, astrocytes may play a role in the pathogenesis of some neurological disease, as well as modulate the severity of neurological damage from injury. As such, an in vitro method of generating stable astrocyte lines would be useful for studying such conditions. Furthermore, astrocytes prepared through methods of in vitro differentiation could be administered therapeutically to patients with neurological disorders, or to reduce the severity of damage in patients who have suffered a neurological injury. However, human pluripotent stem cell (hPSC) derived astrocytes have only been established in vitro after 75-200 days in culture, as such, there are no robust methods for induction of astrocytes from hPSC.

### 3. SUMMARY OF THE INVENTION

The presently disclosed subject matter relates to astrocytes and glial competent cells (e.g., astrocyte precursors), derived from stem cells, e.g., by in vitro differentiation.

The presently disclosed subject matter is based, at least in part, on the discovery that (i) promoting nuclear factor I-A (NFIA) signaling (e.g., increasing expression of NFIA) in a neural stem cell (NSC) initiates a glial competency program. Human embryonic stem cell derived NSC populations include, but are not limited to, those that are derived from dual SMAD inhibition and LTNSCs (also known as “LT-hESCNSCs”, long-term self-renewing rosette-type human embryonic stem cell (ESC) derived neural stem cells). The presently disclosed subject matter is also based, at least in part, on the discovery that (ii) after achieving glial competency, reducing NFIA signaling (e.g., decreasing the expression of NFIA) promotes differentiation of the glial competent cells to astrocytes. Furthermore, exposing the glial competent cells to leukemia inhibitory factor (LIF) (or one or more derivative, analog or activator thereof) promotes differentiation of glial competent cells to astrocytes.

The presently disclosed subject matter is also based, at least in part, on the discovery that (iii) exposing the NSCs, (e.g., Rosette-type NSCs, e.g., LT-hESCNSCs) to fetal bovine serum (FBS) (e.g., in the absence of FGF2) induces differentiations of the NSCs into astrocytes.

The present disclosure provides in vitro methods for inducing differentiation of cells expressing one or more neural stem cell (NSC) marker (e.g., NSCs, e.g., Rosette-type NSCs, e.g., LT-hESCNSCs) into a cell population comprising at least about 10% differentiated cells expressing at least one glial competent cell marker. In certain embodiments, said method comprises promoting NFIA signaling in a population of cells expressing one or more neural stem cell (NSC) marker for an effective period of time to obtain a cell population comprising at least about 10% differentiated cells expressing one or more glial competent cell marker.

In certain embodiments, said method comprises lengthening G1 phase of the cell cycle of a population of cells expressing one or more neural stem cell marker to obtain a cell population comprising at least about 10% differentiated cells expressing one or more glial competent cell marker.

In certain embodiments, the one or more glial competent neural stem cell marker is selected from the group consisting of PAX6, NESTIN, SOX1, SOX2, PLZF, ZO-1, and BRN2. In certain embodiments, the one or more glial competent neural stem cell marker is selected from the group consisting of PAX6, SOX1, PLZF and ZO-1.

The present disclosure also provides in vitro methods for differentiating stem cells (e.g., human stem cells, e.g., pluripotent stem cells) into a cell population comprising at least about 10% differentiated cells expressing one or more glial competent cell marker. In certain embodiments, the method comprises exposing a population of stem cells to an effective amount of one or more inhibitor of SMAD signaling (referred to as “SMAD inhibitor”), and promoting NFIA signaling in the cells to obtain a cell population comprising at least about 10% differentiated cells expressing one or more glial competent cell marker. In certain embodiments, the promotion of NFIA signaling is after or concurrent with the exposure of the cells to the one or more SMAD inhibitor. In certain embodiments, the initial promotion of NFIA signaling is about 2, 3, 4, 5, 6, 7, 8, 9, 10, 11 or 12 days from the initial exposure of the cells to the one or more SMAD inhibitor.

In certain embodiments, the method comprises exposing a population of stem cells to an effective amount of one or more inhibitor of SMAD signaling, and lengthening G1 phase of the cell cycle of the cells, to obtain a cell population comprising at least about 10% differentiated cells expressing one or more glial competent cell marker. In certain embodiments, the initial lengthening of the G1 phase is at least about 8 days from the initial exposure of the cells to the one or more inhibitor of SMAD signaling.

In certain embodiments, the differentiated cells are glial competent cells. In certain embodiments, the glial competent cells are astrocyte precursor cells.

In certain embodiments, said promoting NFIA signaling in the cells comprises increasing expression of NFIA in the cells.

In certain embodiments, increasing expression of NFIA comprises modifying the cells to induce overexpression of NFIA. In certain embodiments, the modified cells express a recombinant NFIA protein, for example, an NFIA nucleic acid wherein expression of said NFIA nucleic acid is operably linked to an inducible promoter.

In certain embodiments, said promoting NFIA signaling in the cells comprises exposing the cells to one or more activator of NFIA (referred to as “NFIA activator”). In certain embodiments, the one or more activator of NFIA comprises an upstream activator of NFIA gene. In certain embodiments, the upstream activator of NFIA gene is TGFβ1. In certain embodiments, the one or more NFIA activator comprises NFIA protein exogenously exposed to the cells.

An effective period of time is a period of time during which a detectable level of at least one glial competent cell marker is achieved, and/or there has been an increase of at least about 10% in the expression level of the at least one glial competent cell marker.

In certain embodiments, the method comprises promoting NFIA signaling in the cells for at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days or more; or for up to about 2 days, for up to about 3 days, for up to about 4 days, for up to about 5 days, for up to about 6 days, for up to about 7 days, for up to about 8 days, for up to about 9 days, for up to about 10 days, for up to about 11 days, for up to about 12 days, for up to about 13 days, for up to about 14 days, for up to about 15 days, for up to about 16 days, for up to about 17 days, for up to about 18 days, for up to about 19 days, for up to about 20 days or more. In certain embodiments, the method comprises promoting NFIA signaling in the cells for about 5 days.

In certain embodiments, the method comprises promoting NFIA signaling in the cells for between about 5 days and about 15 days. In certain embodiments, the method comprises promoting NFIA signaling in the cells for about 8 days. In certain embodiments, the method comprises promoting NFIA signaling in the cells for between about 10 days and about 20 days. In certain embodiments, the method comprises promoting NFIA signaling in the cells for about 15 days.

In certain embodiments, the initial promotion of NFIA signaling is at least about 8 days from the initial exposure of the stem cells to the one or more inhibitor of SMAD signaling. In certain embodiments, a detectable level of the one or more glial competent cell marker is present at least about 5 days from the initial promotion of NFIA signaling in the cells.

In certain embodiments, the glial competent cell marker is selected from the group consisting of CD44, AQP4, SOX2, and NESTIN. In certain embodiments, the glial competent cell marker is selected from the group consisting of CD44, and AQP4.

In certain embodiments, the cell population comprises at least about 10%, about 20%, about 30%, about 40%, about 50%, about 60%, about 70%, about 80%, about 90%, about 95%, about 99% or more of the differentiated cells expressing the one or more glial competent cell marker. In certain embodiments, at least about 99% or more of the differentiated cells express detectable levels of CD44 and at least about 10% differentiated cells express detectable levels of AQP4.

In certain embodiments, the method further comprises exposing the cells to an effective amount of one or more activator of EGF and/or FGF2 signaling. In certain embodiments, the exposure to the one or more activator of EGF and/or FGF2 signaling is concurrent with the promotion of NFIA signaling.

In certain embodiments, the cell population comprises less than about 15% cells expressing a detectable level of one or more neuronal marker. In certain embodiments, the one or more neuronal marker is selected from the group consisting of Tuj1, MAP2, and DCX.

In certain embodiments, the promotion of NFIA signaling in the cells is discontinued, decreased or otherwise inhibited (e.g., by exposure of the cells to an NFIA inhibitor) after about 2, 3, 4, 5, 6, 7, 8, 9, 10, 11, 12, 13, 14, 15, 16, 17, 18, 19, 20 or more days. In certain embodiments, the promotion of NFIA signaling in the cells is discontinued following an about 5-day or about 8-day exposure period. In certain embodiments, the level of expression of functional NFIA is decreased by at least about 10%, about 20%, about 30%, about 40%, about 50%, about 60%, about 70%, about 75%, about 80%, about 85%, about 90%, about 95%, about 98%, about 99% or about 100% compared to the level of expression of functional NFIA initially exposed to the cells. In certain embodiments, the level of expression of functional NFIA is decreased by at least about 10% compared to the level of expression of functional NFIA initially exposed to the cells. In certain embodiments, the level of expression of functional NFIA is decreased by at least about 90% compared to the level of expression of functional NFIA initially exposed to the cells.

In certain embodiments, the promotion of NFIA signaling in the cells is discontinued for a period of time effective to increase a detectable level of expression of one or more astrocyte marker in a plurality of the cells. In certain embodiments, at least 10%, about 20%, about 30%, about 40%, about 50%, about 60%, about 70%, about 80%, about 90%, about 95%, about 99% or more of the cells expresses detectable levels of one or more astrocyte marker. In certain embodiments, at least about 50% or more of the cells expresses detectable levels of one or more astrocyte marker. In certain embodiments, the one or more astrocyte marker is selected from the group consisting of GFAP (Glial fibrillary acidic protein), AQP4 (Aquaporin 4), CD44, S100b (calcium-binding protein B), SOX9 (SRY-Box 9), NFIA, GLT-1, and CSRP1. In certain embodiments, the promotion of NFIA signaling is discontinued or decreased for a period of time effective to decrease a detectable level of expression of SOX2, NESTIN, or both in a plurality of the cells. In certain embodiments, at least about 10%, 20%, 30%, 40%, 50%, 60%, 70%, 80%, 90%, 95%, 99% or more of the cells do not express a detectable level of SOX2, NESTIN, or both. In certain embodiments, at least about 50% or more of the cells do not express a detectable level of SOX2, NESTIN, or both.

In certain embodiments, said effective period of time is at least about 1 day, at least about 2 days, at least about 3 days, at least about 4 days, at least about 5 days, at least about 6 days, at least about 7 days, at least about 8 days, at least about 9 days, at least about 10 days or more; or for up to about 1 day, for up to about 2 days, for up to about 3 days, for up to about 4 days, for up to about 5 days, for up to about 6 days, for up to about 7 days, for up to about 8 days, for up to about 9 days, or for up to about 10 days or more. In certain embodiments, the effective period of time is about 5 days.

In certain embodiments, a detectable level of the one or more glial competent cell marker is present at least about 10 days from the initial lengthening of the G1 phase. In certain embodiments, said lengthening the G1 phase comprises exposing the cells to one or more compound that is capable of lengthening G1 phase of the cell cycle (referred to as “G1 phase lengthening compound”. In certain embodiments, the one or more G1 lengthening compound comprises a small molecule compound. In certain embodiments, the small molecule compound comprises Olomoucine (Olo). In certain embodiments, the method comprises exposing the cells to the one or more G1 lengthening compound for no more than about 2 days.

In certain embodiments, said lengthening the G1 phase comprises increasing expression of FZR1 in the cells.

In certain embodiments, the cells expressing one or more glial competent marker are cortical glial competent cells or spinal glial competent cells.

In certain embodiments, the cells are exposed to the one or more SMAD inhibitor for about 10, 11 or 12 days.

In certain embodiments, the methods further comprising subjecting the cell population comprising at least about 10% cells expressing one or more glial competent cell marker to conditions suitable to promote differentiation of the cells into a cell population comprising at least about 10% cells expressing one or more astrocyte marker. In certain embodiments, the conditions comprise exposing the cells to an effective amount of LIF (or one or more derivative, analog and/or activator thereof), to increase the detectable level of the one or more astrocyte marker. In certain embodiments, the cells are contacted to the effective amount of LIF for at least about 1, 2, 3, 4, 5, 6, 7, 8, 9, 10, 11, 12, 13, 14, 15 days or more; or for up to about 1, 2, 3, 4, 5, 6, 7, 8, 9, 10, 11, 12, 13, 14, 15 or more days.

In certain embodiments, the cells are exposed to the effective amount of LIF for about 7, 8, 9 or 10 days. In certain embodiments, the initial exposure of the cells to LIF, one or more derivative thereof, one or more analog thereof, and/or one or more activator thereof is at least about 10 days from the initial exposure of the stem cells to the one or more inhibitor of SMAD signaling. In certain embodiments, the cells are exposed to the LIF one or more derivative thereof, one or more analog thereof, and/or one or more activator thereof after or concurrently with the promotion of NFIA signaling. In certain embodiments, the initial exposure of the cells to the LIF, one or more derivative thereof, one or more analog thereof, and/or one or more activator thereof is about 1, 2, 3, 4, or 5 days from the initial promotion of NFIA signaling. In certain embodiments, the initial exposure of the cells to LIF, one or more derivative, analog, and/or activator thereof is at least about 2 days or at least about 5 days from the initial promotion of NFIA signaling in the cells.

In certain embodiments, the cells expressing one or more astrocyte marker are cortical astrocytes or spinal cord astrocytes.

Furthermore, the present disclosure provides in vitro methods for differentiating stem cells to a cell population comprising at least about 10% differentiated cells expressing one or more astrocyte marker. In certain embodiments, the method comprises exposing a population of stem cells to an effective amount of one or more inhibitor of SMAD signaling, and exposing the cells to an effective amount of fetal bovine serum (“FBS”) to obtain a cell population comprising at least about 10% differentiated cells expressing one or more astrocyte marker. In certain embodiments, the stem cells are differentiated to said cell population at least about 30 days from the initial exposure of the cells to the FBS.

In certain embodiments, the one or more SMAD inhibitor comprises a TGFβ/Activin-Nodal signaling inhibitor and/or an inhibitor of bone morphogenetic protein (BMP) signaling (referred to as “BMP inhibitor”). In certain embodiments, the one or more inhibitor of TGFβ/Activin-Nodal signaling comprises a compound selected from the group consisting of SB431542, derivatives thereof, and mixtures thereof. In certain embodiments, the one or more inhibitor of TGFβ/Activin-Nodal signaling comprises SB431542. In certain embodiments, the one or more BMP inhibitor comprises a compound selected from the group consisting of LDN193189, derivatives thereof, and mixtures thereof. In certain embodiments, the one or more BMP inhibitor comprises LDN193189.

The presently disclosed subject matter also provides in vitro methods for differentiating stem cells to a cell population comprising at least about 10% differentiated cells expressing one or more spinal cord progenitor marker. In certain embodiments, the method comprises exposing a population of stem cells to an effective amount of one or more SMAD inhibitor, and exposing the cells to an effective amount of one or more activator of retinoic acid (RA) signaling (referred to as “RA activator”) and an effective amount of one or more activator of Sonic hedgehog (SHH) signaling (referred to as “SHH activator”), to obtain a cell population comprising at least about 10% differentiated cells expressing one or more spinal cord progenitor marker.

In certain embodiments, the initial exposure of the cells to the one or more RA activator and the one or more SHH activator at least about one day from the initial exposure of the cells to the one or more SMAD inhibitor.

In certain embodiments, a detectable level of the one or more spinal cord progenitor marker is present at least about 12 days from the initial exposure of the cells to the one or more RA activator and the one or more SHH activator. In certain embodiments, the one or more spinal cord progenitor marker is selected from the group consisting of HOXB4, ISL1, and NKX6.1.

In certain embodiments, the stem cells are human stem cells. In certain embodiments, the stem cells are pluripotent stem cells. In certain embodiments, the pluripotent stem cells are selected from the group consisting of embryonic stem cells, induced pluripotent stem cells, and combinations thereof. In certain embodiments, said human stem cells are selected from the group consisting of human embryonic stem cells, human induced pluripotent stem cells, human parthenogenetic stem cells, primordial germ cell-like pluripotent stem cells, epiblast stem cells, and F-class pluripotent stem cells, and combinations thereof. In certain embodiments, the stem cells are NSCs. In certain embodiments, the stem cells are Rosette-type NSCs. In certain embodiments, the stem cells are LT-NSCs.

The presently disclosed subject matter further provides a population of in vitro differentiated cells expressing at least about 10% one or more glial competent cell marker and/or one or more astrocyte marker, wherein said differentiated cell population is derived from the method disclosed herein. The presently disclosed subject matter further provides compositions comprising said cell population. In certain embodiments, the composition is a pharmaceutical composition.

Furthermore, the presently disclosed subject matter provides kits for inducing differentiation of stem cells. In certain embodiments, the kit comprises one or more of the following: (a) one or more inhibitor of TGFβ/Activin-Nodal signaling, (b) one or more BMP inhibitor, (c) one or more NFIA activator, (d) LIF, one or more derivative, analog, and/or activator thereof, (e) FBS, and (f) instructions for inducing differentiation of the stem cells into a population of differentiated cells that express one or more astrocyte marker, and/or one or more glial competent cell marker.

The presently disclosed subject matter further provides a composition comprising a population of in vitro differentiated cells, wherein at least about 50% of the population of cells express one or more NSC marker and wherein less than about 25% of the population of cells express one or more stem cell marker. The presently disclosed subject matter also provides a composition comprising a population of in vitro differentiated cells, wherein at least about 50% of the population of cells express one or more glial competent cell marker or glial competent cell marker, and wherein less than about 25% of the population of cells express one or more marker selected from the group consisting of stem cell markers, NSC markers, and neuronal markers. The presently disclosed subject matter also provides composition comprising a population of in vitro differentiated cells, wherein at least about 50% of the population of cells express one or more astrocyte marker, and wherein less than about 25% of the population of cells express one or more marker selected from the group consisting of stem cell markers, NSC markers, neuronal markers, and glial competent NSC markers/glial competent cell markers.

In certain embodiments, the one or more stem cell marker is selected from the group consisting of OCT4, NANOG, SOX2, LIN28, SSEA4 and SSEA3. In certain embodiments, the one or more neural stem cell (NSC) marker is selected from the group consisting of PAX6, NESTIN, SOX1, SOX2, PLZF, ZO-1, and BRN2. In certain embodiments, the one or more neural stem cell marker is selected from the group consisting of PAX6, SOX1, PLZF, and ZO-1. In certain embodiments, the one or more glial competent cell marker is selected from the group consisting of CD44, AQP4, SOX2, and NECTIN. In certain embodiments, the one or more glial competent cell marker is selected from the group consisting of CD44 and AQP4. In certain embodiments, the one or more astrocyte marker is selected from the group consisting of GFAP, AQP4, CD44, S100b, SOX9, NFIA, GLT-1 and CSRP1. In certain embodiments, the one or more neuronal marker is selected from the group consisting of Tuj1, MAP2, and DCX.

The presently disclosed subject matter further provides methods of treating a neurodegenerative disorder in a subject, or for reducing damage due to neurological injury, for example, ischemia or stroke, in a subject. In certain embodiments, the method comprises administering the subject the differentiated cell population described herein or the composition described herein. In certain embodiments, the subject suffers from a neurodegenerative disorder and/or has experienced a neurological injury.

The presently disclosed subject matter further provides a differentiated cell population described herein or a composition comprising thereof for treating a neurodegenerative disorder in a subject in need thereof, or for reducing damage due to neurological injury. In certain embodiments, the subject has been diagnosed with or at risk of having a neurodegenerative disorder.

The presently disclosed subject matter further provides uses of the differentiated cell population described herein or the composition described herein in the manufacture of a medicament for treating a neurodegenerative disorder, for reducing damage from a neurological injury, or to reduce severity of damage due to neurological injury, such as stroke.

In certain embodiments, the neurodegenerative disorder is Parkinson's disease, Alzheimer's disease, amyotrophic lateral sclerosis (ALS), or Rett syndrome.

A1. In certain non-limiting embodiments, the presently disclosed subject matter provides an in vitro method for differentiating stem cells, comprising exposing a population of stem cells to an effective amount of one or more inhibitor of SMAD signaling, and one or more activator of NFIA, to obtain a cell population comprising at least about 10% differentiated cells expressing one or more glial competent cell marker.

A2. The method of A1, wherein the population of stem cells are initially exposed to the one or more activator of NFIA at least about 8 days from the initial exposure of the cells to the one or more inhibitor of SMAD signaling.

A3. The method of A2, wherein a detectable level of the one or more glial competent cell marker is present at least about 5 days from the initial exposure of the cells to the one or more activator of NFIA, optionally wherein the one or more glial competent cell marker is selected from the group consisting of CD44, AQP4, SOX2, and NESTIN.

A4. The method of A3, wherein the level of expression of functional NFIA activity is decreased in the plurality of cells after the presence of a detectable level of the one or more glial competent cell marker, after which the cells are cultured under conditions to promote differentiation of the cells into cells expressing one or more astrocyte marker.

A5. The method of A4, wherein the one or more astrocyte marker is selected from the group consisting of GFAP, AQP4, CD44, S100b, SOX9, NFIA, GLT-1, and CSRP1.

A6. The method of A5, wherein the one or more astrocyte marker comprises GFAP.

A7. The method of A4, wherein the level of expression of functional NFIA activity is decreased by at least about 90%.

A8. The method of A1, wherein the cells exposed to the one or more activator of NFIA do not express a detectable level of one or more neuronal marker, optionally wherein the one or more neuronal marker is selected from the group consisting of Tuj1, MAP2, and DCX.

A9. The method of A1, further comprising exposing the cells to leukemia inhibitory factor (LIF), one or more derivative thereof, one or more analog thereof, and/or one or more activator thereof.

A10. The method of A9, wherein the initial exposure of the cells to LIF, one or more derivative thereof, one or more analog thereof, and/or one or more activator thereof is at least about 10 days from the initial exposure of the cells to the one or more inhibitor of SMAD signaling.

A11. The method of A1, wherein the one or more inhibitor of SMAD signaling comprises one or more inhibitor of transforming growth factor beta (TGFβ)/Activin-Nodal signaling, and one or more inhibitor of bone morphogenetic protein (BMP) signaling.

A12. The method of A11, wherein the one or more inhibitor of TGFβ/Activin-Nodal signaling comprises a compound selected from the group consisting of SB431542, derivatives thereof, and mixtures thereof.

A13. The method of A11, wherein the one or more inhibitor of bone morphogenetic protein (BMP) signaling comprises a compound selected from the group consisting of LDN193189, derivatives thereof, and mixtures thereof.

A14. The method of A1, wherein the one or more activator of NFIA comprises NFIA protein exogenously exposed to the stem cells.

A15. The method of A1, wherein the one or more activator of NFIA comprises a recombinant NFIA protein expressed by the stem cells.

A16. In certain non-limiting embodiments, the presently disclosed subject matter provides an in vitro method for differentiating stem cells comprising exposing a population of stem cells to an effective amount of one or more inhibitor of SMAD signaling, and fetal bovine serum, to obtain cell population comprising at least about 10% differentiated cells expressing one or more astrocyte marker.

A17. The method of A16, wherein the stem cells are differentiated to said cell population at least about 30 from the initial exposure of the cells to the fetal bovine serum.

A18. The method of any preceding method, wherein the stem cells are human stem cells.

A19. The method of any preceding method, wherein the stem cells are pluripotent stem cells.

A20. The method of any preceding method, wherein the stem cells are selected from the group consisting of human embryonic stem cells, human induced pluripotent stem cells, human parthenogenetic stem cells, primordial germ cell-like pluripotent stem cells, epiblast stem cells, and F-class pluripotent stem cells.

A21. In certain non-limiting embodiments, the presently disclosed subject matter provides an in vitro method for differentiating pluripotent stem cells, comprising exposing a population of cells expressing one or more glial competent neural stem cell marker to an effective amount of one or more activator of NFIA, to obtain a cell population comprising at least about 10% differentiated cells expressing one or more glial competent cell marker.

A22. The method of A21, wherein a detectable level of the one or more glial competent cell marker is present at least about 5 days from the initial exposure of the cells to the one or more activator of NFIA, optionally wherein the one or more glial competent cell marker is selected from the group consisting of CD44, AQP4, SOX2, and NESTIN.

A23. The method of A22, wherein the level of expression of functional NFIA activity is decreased in the plurality of cells after a detectable level of the one or more glial competent cell marker is expressed by the plurality of cells, after which and the cells are cultured under conditions to promote differentiation of the cells into cells expressing one or more astrocyte marker.

A24. The method of A23, wherein the one or more astrocyte marker is selected from the group consisting of GFAP, AQP4, CD44, S100b, SOX9, NFIA, GLT-1, and CSRP1.

A25. In certain non-limiting embodiments, the presently disclosed subject matter provides a cell population comprising at least about 10% in vitro differentiated cells expressing one or more astrocyte marker, and/or one or more glial competent cell marker, optionally wherein said cell population is obtained by the methods of any one of A1-24.

A26. In certain non-limiting embodiments, the presently disclosed subject matter provides a composition comprising the cell population of A25, optionally the composition is a pharmaceutical composition.

A27. In certain non-limiting embodiments, the presently disclosed subject matter provides a method of treating a neurodegenerative disorder, or reducing damage from a neurological injury, in a subject, comprising administering an effective amount of the population of in vitro differentiated cells according to A25 or the composition of A26 into a subject in need thereof.

A28. The method of A27, wherein the subject has been diagnosed with or at risk of having a neurodegenerative disorder.

A29. The method of A28, wherein the neurodegenerative disorder is selected from the group consisting of Parkinson's disease, Alzheimer's disease, amyotrophic lateral sclerosis (ALS), and Rett syndrome.

A30. In certain non-limiting embodiments, the presently disclosed subject matter provides use of the population of in vitro differentiated cells according to A25 or the composition of A26 in the manufacture of a medicament for treating a neurodegenerative disorder or for reducing damage from a neurological injury.

A31. In certain non-limiting embodiments, the presently disclosed subject matter provides a kit for inducing differentiation of stem cells, comprising one or more of:

(a) one or more inhibitor of transforming growth factor beta (TGFβ)/Activin-Nodal signaling,

(b) one or more inhibitor of BMP signaling;

(c) one or more activator of NFIA;

(d) LIF, a derivative thereof, analog thereof, and/or activator thereof; and

(e) instructions for inducing differentiation of the stem cells into a cell population comprising at least about 10% differentiated cells expressing one or more glial competent cell marker and/or one or more astrocyte marker.

A32. In certain non-limiting embodiments, the presently disclosed subject matter provides a kit comprising a cell population of A25.

### 4. BRIEF DESCRIPTION OF THE DRAWINGS

FIG. 1A-1H shows that dual SMAD inhibition generated a highly homogenous neural stem cell population of long-term self-renewing rosette-type human embryonic stem cell (ESC) derived neural stem cells (LTNSCs). FIG. 1A shows schematic of differentiation strategy to generate LTNSCs. Depiction of the dual SMAD culture protocol used to differentiate hESCs into LTNSCs, wherein the hESCs were differentiated into neural stem cells (NSC) followed by culture in FGF2/EGF. FIG. 1B shows the morphology of the LTNSCs resembles a very early neuroectoderm. FIG. 1C shows immunofluorescence of SOX2, NESTIN, SOX1, ZO-1 and PLZF in LTNSCs (p20) compared to a glial competent NSCs (NSCEGF/FGF). The LTNSCs express neural stem cell (NSC) markers SOX2, NESTIN and SOX1, and also express PLZF and focal expression of ZO-1, markers of rosette or early NSC development. FIG. 1D shows Immunofluorescence staining of LTNSCs (p3 and p20) and NSCEGF/FGF for regional markers FOXG1 and OTX2. Sustained culture of the LTNSCs resulted in increased expression of forebrain markers OTX2 and FOXG1, followed by FIG. 1E more caudal and ventral markers such as GBX2 and NKX2.1, respectively. FIG. 1E shows bar chart of quantitative PCR analysis of additional regional markers in LTNSCs (p20). FIG. 1F shows immunofluorescence of βIII-Tubulin and GFAP during the differentiation of LTNSCs and glial competent NSCs. Sustained culture of the LTNSCs resulted in differentiation into neurons expressing the neuronal marker TUJ1. FIG. 1G shows immunofluorescence of SOX2, NES, ZO-1, β-tubulin, and PLZF in LTNSCs (p30). FIG. 1H shows immunofluorescence of CD44, NFIA, GFAP, AQO4, SOX2, NESTIN, and TUJ1 shows that LTNSCs are a highly homogenous population.

FIG. 2A-B shows that FIG. 2A shows the LTNSC express stem cell markers SOX2 and NESTIN, but do not express glial competency markers (i.e., astrocyte precursor markers) CD44, GFAP, and AQP4, or the neural marker TUJ1. FIG. 2B shows immunofluorescence of βIII-Tubulin and GFAP as LTNSCs are treated with different factors for 14 days. Culturing the LTNSCs in media containing gliogenic molecules LIF, BMP4 or FBS increased expression of the neuronal marker TUJ1 rather than the astrocyte marker GFAP.

FIGS. 3A-3E. Serum factors accelerate the onset of gliogenesis from human NSCs. FIG. 3A shows Diagram depicting the differentiation strategy and time course analysis of NSC differentiation. FIG. 3B shows immunofluorescence analysis of GFAP, MAP2 and AQP4::H2B GFP after 30 days of treatment in 1% FBS. Scale bars are 50 μm. FIG. 3C shows diagram depicting the differentiation strategy and time course analysis of NSC differentiation. FIG. 3D shows that culturing neural rosettes or LTNSCs in FBS for an extended time period of about 30 days increased expression of glial competency markers AQP4 and GFAP compared to culture with no growth factors, Notch inhibitors, LIF, or BMP4. FIG. 3E shows that, additionally, the FBS treated cells also expressed higher levels of gliogenic factors SOX9 and NFIA compared to the other treatments.

FIG. 4A-C. FIG. 4A shows overexpression of NFIA was achieved by viral infection of LTNSCs with a doxycycline inducible vector comprising the NFIA cDNA. FIG. 4B shows after 7 days of doxycycline treatment, there was a large morphological change in the cell population where the NFIA induced cells expressed CD44. Expression of the astrocyte marker GFAP was observed when the cells were cultured in FBS. FIG. 4C shows after induced expression of NFIA was discontinued, culturing the cells in LIF, BMP4, and FBS enhanced the expression of GFAP especially in the absence of FGF2.

FIG. 5 shows that NFIA allowed for acquisition of glial competency but was inhibitory to glial differentiation. Increasing NFIA expression in the absence of FBS did not increase expression of the astrocyte marker GFAP. However, a subsequent decrease of NFIA expression and culture in LIF increased the expression of GFAP.

FIGS. 6A-6K shows that NFIA allowed for acquisition of glial competency but cannot maintain the glial competent state. FIG. 6A shows FACS plot of CD44 expressing cells treated with continuous doxycycline or doxycycline removed demonstrates that CD44 expression is lost after doxycycline removal. FIG. 6B shows immunofluorescence staining for NFIA, GFAP and TUBB3 in NSCs, NFIA-induced NSCs and NFIA-induced NSCs with doxycycline removal. FIG. 6C shows schematic representation of cells induced with NFIA and attaining glial competency then reversal to glial incompetency with doxycycline withdrawal. FIG. 6D shows quantitative PCR data of NFIA expression along the timecourse represented in C. FIG. 6E shows sample distance plot for RNA expression of NSCs at different timepoints related C. FIG. 6F shows sample distance plot for chromatin accessibility compared to glial competent NSCs (gcNSCs) and hPSC-derived astrocytes (200 days of in vitro culture). FIG. 6G shows example ATAC-seq tracks at the GFAP locus that depicts the lack of chromatin accessibility in several NSC samples. FIG. 6H shows bisulfite sequencing of the promoter region of the GFAP promoter suggests that CD44 positive cells resulting from overexpression of NFIA leads to demethylation of a specific CpG on the GFAP promoter. Error bars are calculated by S.E.M. FIG. 6I shows schematic showing that GFAP promoter is methylated in the neurogenic state. FIG. 6J shows increasing NFIA expression induced a corresponding increase in CD44 expression, a marker of glial competent cells. FIG. 6K shows GFAP expression in neurogenic cells is blocked by methylation at the STAT3 CpG site (arrow), while this site is demethylated in glial cells (arrowhead), wherein GFAP is expressed. The STAT3 CpG site is demethylated in CD44+ cells induced by NFIA (arrowhead). FIG. 6L shows brightfield shows morphology of cells treated with continuous doxycycline or doxycycline removed demonstrates. FIG. 6M shows immunofluorescence staining of DAPI, GFAP and NFIA in NSCs, NFIA-induced NSCs and NFIA-induced NSCs with doxycycline removal. FIG. 6N shows FACS plot of CD44 expressing cells treated with continuous doxycycline or doxycycline removed demonstrates that CD44 expression is lost after doxycycline removal. FIG. 6O shows immunofluorescence staining of DAPI, GFAP, NFIA, AQP4, and CD44 in NSCs, NFIA-induced NSCs and NFIA-induced NSCs with doxycycline removal. FIG. 6P shows immunofluorescence staining of DAPI, NFIA, and TUJ1 in LTNSCs, NFIA-induced NSCs with doxycycline removal, and NFIA-induced NSCs with doxycycline continuous present. LTNSCs regain their neurogenic potential after continuous NFIA activation. FIG. 6Q shows NFIA overexpression top motifs and astrocyte top motifs associated with FIG. 6F.

FIG. 7 shows a strategy for culturing a recombinant NFIA-inducible hESC line under the control of doxycycline into astrocytes, wherein the cells were cultured with dual SMAD inhibition to generate neural stem cells, followed by an increase in NFIA expression that induced differentiation of glial competent cells expressing CD44 and GFAP, followed by downregulation of NFIA expression and culture in LIF that induced differentiation of the cells to astrocytes expressing GFAP, CD44 and AQP4.

FIGS. 8A-8E. No-limiting examples of protocols for the generation of NFIA-induced astrocytes. FIG. 8A shows schematic diagram of the protocol to induce astrocytes from NSCs using transient NFIA expression compared to FIG. 8B, which is a summary of current protracted glial differentiation strategies from hPSCs. FIGS. 8C-8E shows non-limiting examples of protocols for FIG. 8C differentiating stem cells (LTNSCs) into astrocytes by culturing the cells in FBS, or FIG. 8D modulating the expression of NFIA in combination with culture in LIF. FIG. 8E shows no-limiting examples of protocols for the generation of NFIA-induced astrocytes.

FIGS. 9A-9C. FIG. 9A shows schematic of the culturing protocol. FIG. 9B shows culturing oMN with wild type astrocytes increased survival of the motor neurons compared to culture with SOD1A4V astrocytes, while the wild type astrocytes reduced cell death of sMN compared to SOD1A4V astrocytes. FIG. 9C shows immunofluorescent staining of VACHT+ cells.

FIGS. 10A-10L. Transient expression of NFIA in neuroepithelial stem cells endows glial competency. FIG. 10A shows quantitative PCR of candidate genes associated with glial competency treated in serum conditions for 30 days (n=3), p-value <0.01 (NFIA) and p-value <0.015 (LIN28B) was calculated using a paired t-test comparing serum and N2. FIG. 10B shows overexpression of NFIA leads to profound morphological changes within 5 days of doxycycline treatment marked by yellow arrowheads. FIG. 10C shows immunofluorescence staining of NFIA (red), GFAP (green) and CD44 (far red) in NSCs treated with doxycycline for 5 days. FIG. 10D shows immunofluorescence staining of the AQP4-H2B-GFP reporter and SOX2 in NSCs treated with doxycycline for 5 days. FIG. 10E shows quantitative PCR analysis of GFAP and NFIA expression in NSCs treated with doxycycline for 5 days and subsequent removal for an additional 3 and 5 days or continuous treatment (+dox) (n=2). FIG. 10F shows immunofluorescence staining of GFAP in NSCs transiently induced with NFIA and subsequently maintained in either FGF2 or LIF containing media. FIG. 10G shows brightfield image of a representative astrocyte (60 days after CD44 sort). FIG. 10H shows immunofluorescence staining of GFAP and SLC1A2 in d60 astrocyte culture. FIG. 10I shows quantitative PCR analysis of genes associated with NSCs, neurons, astrocytes and oligodendrocytes from NFIA-induced astrocytes (n=2). FIG. 10J shows quantitative PCR analysis of genes associated with fetal and adult astrocytes in multiple derivations and passages of NFIA-induced astrocytes with primary human astrocytes (n=2). Scale bars are 50 sum. Error bars are calculated by S.E.M. FIG. 10K shows immunofluorescence staining of NFIA (red), GFAP (green) and CD44 (far red) in NSCs treated with doxycycline for 4 days. FIG. 10L shows immunofluorescence staining of the AQP4-H2B-GFP reporter, SOX, NIFA and GFAP in NSCs treated with doxycycline for 5 days.

FIGS. 11A-11K. NFIA-induced astrocytes are functional. FIG. 11A shows immunofluorescence staining of neurons either cultured alone or co-cultured with NFIA-induced astrocytes. FIG. 11B shows western blot analysis of markers of maturity, MUNC13.1 and synapsin I in neurons cultured with or without astrocytes. FIG. 11C shows a bar chart representing cell survival after glutamate excitotoxicity assays in cultures similar to B. P-value (p-value <0.0001) calculated with a t-test (n=3) and error bars are calculated by S.E.M. FIG. 11D shows bar chart representing the amount of complement (C3) released from NFIA-induced or primary astrocytes treated with IL1α, TNF and C1q for 24 hours (n=2). FIG. 11E shows ratiometric plots of purified NFIA-induced astrocytes (60 days) incubated with the Fura-2 calcium dye and stimulated with ATP, KCl and Glutamate. All data points are plotted as a heatmap below. FIG. 11F shows FACS histograms of AQP4-H2B-GFP intensities in astrocytes only cultures (red) and astrocytes co-cultured with neurons (blue) with the GFP negative fraction plotted in the histogram below. FIG. 11G shows ratiometric plots of NFIA-induced astrocytes co-cultured with neurons incubated with the Fura-2 calcium dye and stimulated with ATP, KCl and Glutamate. Ratios were calculated on GFP positive nuclei. All data points are plotted as a heatmap below. FIG. 11H shows quantification of the number of astrocytes responding to ATP, KCl or Glutamate from data presented in FIG. 11E, F and Extended Data FIG. 7. FIG. 11I shows immunofluorescence of NFIA-induced glial progenitors transplanted into the mouse cortex depict migration through the corpus callosum. Scale bar 50 μm FIG. 11J shows immunofluorescence of NFIA-induced astrocytes demonstrate co-expression of AQP4-H2B-GFP, GFAP and the human specific marker SC-121. Scale bar 10 μm. FIG. 11K shows immunofluorescence image of a long human astrocyte process. Scale bar 5 μm.

FIGS. 12A-12N. NFIA induces a slower G1 cell cycle phase to induce glial competency. FIG. 12A shows unbiased hierarchical clustering of genes during the timecourse with three major clusters highlighted. FIG. 12B shows gene ontology analysis of the significant biological processes from each cluster represented in A. FIG. 12C shows global analysis of all genes and genes specifically in the cell cycle ontology. P-value calculated using the hypergeometric distribution. FIG. 12D shows graph of expression dynamics for all cell division cycle (CDC) genes during the timecourse. FIG. 12E shows cell cycle analysis by FACS on NSCs with or without dox for 7 days. FIG. 12F shows western blot analysis of CCNA1 and CDKN1A in LTNSCs with or without dox for 7 days. FIG. 12G shows quantitative PCR assessing the knockdown efficiency of shFZR1 in the presence of NFIA over expression (n=3). FIG. 12H shows analysis of the cell cycle phase with or without shRNA to FZR1. FIG. 12I shows quantitative PCR analysis of CD44 expression in NFIA induced cells with or without shFZR1 (n=3). FIG. 12J shows quantitative PCR analysis of GFAP expression in NFIA induced cells similar to I but further induced with LIF (n=2). FIG. 12K shows Western blot analysis of NFIA when induced with various levels of TGFβ1 for 7 days. (L) Cell cycle analysis by FACS on NSCs treated with or without TGFβ1 for 48 hours. FIG. 12M shows immunofluorescence staining for NFIA and GFAP on cultures treated with or without TGFβ1 for 14 days. FIG. 12N shows model of NFIA-induced glial competency. Scale bars are 50 μm.

FIGS. 13A-13I. Generation and characterization of an aquaporin-4 knock-in hESC reporter line. FIG. 13A shows schematic of knock-in strategy to incorporate an H2B-GFP reporter line into the AQP4 locus. FIG. 13B shows genomic PCR confirmation of heterozygous knock-in. FIG. 13D shows immunofluorescence of OCT4 and SOX2 in AQP4-H2B-GFP hESCs. FIG. 13C shows immunofluorescence of PLZF and PAX6 as the AQP4-H2B-GFP line is differentiated toward the neuroectoderm. FIG. 13E shows immunofluorescence of AQP4 and GFP as the AQP4-H2B-GFP line is differentiated in long term cultures toward astrocytes. FIG. 13F shows same as in FIG. 13E but immunofluorescence of GFP and βIII-Tubulin. Same as FIG. 13E but immunofluorescence of GFP and GFAP. FIG. 13H shows similar as FIG. 13E immunofluorescence of GFP and SOX2. Scale bars are 50 μm. FIG. 13I shows brightfield and immunofluorescence of AQP4 and GFP as the AQP4-H2B-GFP line is differentiated in more than 100 days cultures toward astrocytes.

FIGS. 14A-14D. LIF promotes efficient differentiation towards astrocytes. FIG. 14A shows immunofluorescence analysis of GFAP expression after NFIA induced cells were treated with BMP4 and 1% FBS. FIGS. 14B and 14C show immunofluorescence staining of NFIA expression and the AQP4-H2B-GFP reporter signal upon treatment with FGF2/EGF, FGF2, HB-EGF, BMP4, 1% FBS and LIF after NFIA-induction. Scale bars are 100 μm. FIG. 14D shows NFIA and other glial related proteins are not present at the LTNSC stage.

FIGS. 15A-15D. NFIA-induction is applicable to forebrain and spinal cord patterned NSCs. FIGS. 15A and 15C show quantitative PCR measurements of anterior markers OTX2, FOXG1, PAX6 and posterior markers HOXB4, FOXA2, and NKX6.1 after the differentiation of several hPSCs towards the neuroectoderm (day 10 from pluripotency). FIG. 15B shows immunofluorescence of OTX2, NFIA and GFAP after NFIA transient expression of NFIA (day 18 from pluripotency). Error bars are calculated by S.E.M. FIG. 15D shows non-limiting examples of protocol for inducing astrocytes from different regionally patterned progenitors.

FIGS. 16A-16F. Calcium imaging of primary astrocytes, hPSC-derived progenitors and astrocytes. FIG. 16A shows brightfield and GFP image of late NSC population derived from the AQP4-H2B-GFP reporter line. FIG. 16B shows timecourse of calcium imaging of hPSC-derived NSCs treated with KCl, Glutamate and ATP, each grey line represents an individual cell trace. Black line represents the mean signal. FIG. 16C shows brightfield and GFP image of AQP4::H2B-GFP sorted astrocytes. FIG. 16D is similar to FIG. 16B but with hPSC-derived astrocytes (d120). (E) Quantification of the number of cells responding to particular stimuli. FIG. 16F is similar to FIG. 16B with commercially available primary astrocytes. Each line represents a cell (top). Heatmap of all cells analyzed in a heatmap format (bottom).

FIGS. 17A-17C. ATAC-seq motif analysis shows enrichment of NFI-motifs. FIG. 17A shows immunofluorescence staining of NFIA and GFAP in LTNSCs with continuous doxycycline (dox) treatment (left) or after dox was removed (right). Scale bars are 50 μm. FIG. 17B shows ATAC-seq tracks at the SOX2 locus displaying the open chromatin. FIG. 17C shows motif analysis of ATAC-seq peaks in the four conditions; values on the X-axis represent the −log 10 p-value. FACS analysis and sorting of CD44 positive and negative fraction for bisulfite sequencing. Scale bars are 50 sum.

FIGS. 18A-18B. Analysis of Group I and II clusters during transient NFIA-activation. FIG. 18A shows Group I highlights gene expression changes related to markers of fetal astrocytes. FIG. 18B shows Group II, emphasizing gene expression changes related to induction of growth factor related genes.

FIGS. 19A-19B. Gene expression changes in Cyclin related genes. FIG. 19A shows bar chart indicating the expression patterns of Cyclin genes during transient NFIA activation. FIG. 19B shows quantitative PCR validation of CCNA1 upregulation due to NFIA overexpression (n=3).

FIGS. 20A-20E. Altered astrocyte differentiation potential due to titration of NFIA. FIG. 20A shows quantitative PCR of NFIA expression over a timecourse of differentiation (n=2).

FIG. 20B shows G1 timing analysis of LTNSCs and LTNSCs induced with NFIA using the FUCCI-O reporter construct. FIG. 20C shows bar chart of cell cycle profiles during a dox titration for 5 days. FIG. 20D shows quantitative PCR for the expression of NFIA, CD44 and GFAP during the titration of NFIA expression (n=3). FIG. 20E shows immunofluorescence of GFAP and NFIA expression in LTNSCs treated with varying concentration of dox after 10 days. Scale bars are 50 sum. Error bars are calculated by S.E.M.

FIGS. 21A-21F. Chemically induced G1 lengthening by Olomoucine can upregulate glial competent gene expression. FIG. 21A shows FACS analysis of the percentage of cells in the G1 phase treated with Olomoucine. FIG. 21B shows quantitative PCR analysis of NFIA, CD44 and S100β after the treatment with Olomoucine after 48 hours (n=2). FIG. 21C shows quantitative PCR analysis of NFIA, CD44 and S100β expression after LTNSCs were treated with or without Olomoucine for 12 days (n=3). FIG. 21D shows immunofluorescence staining of GFAP on cells treated with LIF and Olomoucine after 12 days. FIG. 21E shows FACS analysis of LTNSCs treated with 1% FBS demonstrating a G1 arrest. FIG. 21F shows immunofluorescence staining of NFIA and CD44 in FBS treated LTNSCs with or without Olomoucine. Scale bars are 50 μm. Error bars are calculated by S.E.M.5.

### DETAILED DESCRIPTION

The presently disclosed subject matter as described herein relates to methods of preparing glial competent cells (e.g., “astrocyte precursors) and/or astrocytes derived from stem cells, and methods for producing such cells. Also provided are uses of such cells for treating a neurodegenerative disorder.

For purposes of clarity of disclosure and not by way of limitation, the detailed description is divided into the following subsections:

5.1 Definitions;

5.2 Methods of Differentiating Stem Cells;

5.3 In Vitro Differentiation of Stem Cells to Regional Astrocytes

5.4 Compositions Comprising Differentiated Cell Populations;

5.5 Methods of Preventing and/or Treating Neurodegenerative Disorders; and

5.6 Kits

### 5.1 Definitions

The terms used in this specification generally have their ordinary meanings in the art, within the context of this invention and in the specific context where each term is used. Certain terms are discussed below, or elsewhere in the specification, to provide additional guidance to the practitioner in describing the compositions and methods of the invention and how to make and use them.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold, or within 2-fold, of a value.

As used herein, the term “neural stem cells” or “NSCs” refers to stem cells that are neurogenic and have not undergone gliogenic switch. In certain embodiments, the NSCs are Rosette-stage neural stem cells. In certain embodiments, the NSCs are Long-term NSCs (LTNSCs). In certain embodiments, the NSCs express one or more neural stem cell marker. Non-limiting examples of neural stem cell markers include PAX6, NESTIN, SOX1, SOX2, PLZF, ZO-1, and BRN2. In certain embodiments, the neural stem cell marker is selected from the group consisting of PAX6, SOX1, PLZF, ZO-1, and combinations thereof,

As used herein, the terms “LTNSC” or “LT-hESCNSCs” refer to long-term self-renewing Rosette-type human embryonic stem cell (ESC) derived neural stem cells). The morphology of the LTNSCs resemble a very early neuroectoderm. LTNSCs express several neural stem cell (NSC) markers such as SOX2, NESTIN and SOX1 but also express PLZF and display a focal expression of ZO-1 indicating their rosette or early NSC nature.

As used herein, the term “glial competence” refers to the competence of a NSC directed towards glial differentiation.

As used herein, the term “glial competent cells” refers to cells that have undergone gliogenic switch and have possessed glial competence. In certain embodiments, the glial competent cells express one or more glial competent cell marker. Non-limiting examples of glial competent cell markers include CD44, AQP4, SOX2, and NECTIN. In certain embodiments, the glial competent cell marker is selected from the group consisting of CD44, AQP4, and a combination thereof. In certain embodiments, the glial competent cells are astrocyte precursors.

As used herein, the term “signaling” in reference to a “signal transduction protein” refers to a protein that is activated or otherwise affected by ligand binding to a membrane receptor protein or some other stimulus. Examples of signal transduction protein include, but are not limited to, a SMAD, transforming growth factor beta (TGFβ), Activin, Nodal, bone morphogenic (BMP) and NFIA proteins. For many cell surface receptors or internal receptor proteins, ligand-receptor interactions are not directly linked to the cell's response. The ligand activated receptor can first interact with other proteins inside the cell before the ultimate physiological effect of the ligand on the cell's behavior is produced. Often, the behavior of a chain of several interacting cell proteins is altered following receptor activation or inhibition. The entire set of cell changes induced by receptor activation is called a signal transduction mechanism or signaling pathway.

As used herein, the term “signals” refer to internal and external factors that control changes in cell structure and function. They can be chemical or physical in nature.

As used herein, the term “ligands” refers to molecules and proteins that bind to receptors, e.g., transforming growth factor-beta (TFGβ), Activin, Nodal, bone morphogenic proteins (BMPs), etc.

“Inhibitor” as used herein, refers to a compound or molecule (e.g., small molecule, peptide, peptidomimetic, natural compound, siRNA, anti-sense nucleic acid, aptamer, or antibody) that interferes with (e.g., reduces, decreases, suppresses, eliminates, or blocks) the signaling function of the molecule or pathway. An inhibitor can be any compound or molecule that changes any activity of a named protein (signaling molecule, any molecule involved with the named signaling molecule, or a named associated molecule) (e.g., including, but not limited to, the signaling molecules described herein). For one example, an inhibitor of SMAD signaling can function, for example, via directly contacting SMAD, contacting SMAD mRNA, causing conformational changes of SMAD, decreasing SMAD protein levels, or interfering with SMAD interactions with signaling partners, and affecting the expression of SMAD target genes. Inhibitors also include molecules that indirectly regulate SMAD biological activity by intercepting upstream signaling molecules (e.g., within the extracellular domain). Examples of a SMAD signaling inhibitor molecules and an effect include: Noggin which sequesters bone morphogenic proteins, inhibiting activation of ALK receptors 1,2,3, and 6, thus preventing downstream SMAD activation. Likewise, Chordin, Cerberus, Follistatin, similarly sequester extracellular activators of SMAD signaling. Bambi, a transmembrane protein, also acts as a pseudo-receptor to sequester extracellular TGFβ signaling molecules. Antibodies that block activins, nodal, TGFβ, and BMPs are contemplated for use to neutralize extracellular activators of SMAD signaling, and the like. Although the foregoing example relates to SMAD signaling inhibition, similar or analogous mechanisms can be used to inhibit other signaling molecules. Examples of inhibitors include, but are not limited to: LDN193189 (LDN) and SB431542 (SB) (LSB) for SMAD signaling inhibition.

Inhibitors are described in terms of competitive inhibition (binds to the active site in a manner as to exclude or reduce the binding of another known binding compound) and allosteric inhibition (binds to a protein in a manner to change the protein conformation in a manner which interferes with binding of a compound to that protein's active site) in addition to inhibition induced by binding to and affecting a molecule upstream from the named signaling molecule that in turn causes inhibition of the named molecule. An inhibitor can be a “direct inhibitor” that inhibits a signaling target or a signaling target pathway by actually contacting the signaling target.

“Activators”, as used herein, refer to compounds that increase, induce, stimulate, activate, facilitate, or enhance activation of a protein or molecule, or the signaling function of the protein, molecule or pathway, e.g., activating NFIA transcription factor activity.

As used herein, the term “derivative” refers to a chemical compound with a similar core structure.

As used herein, the term “a population of cells” or “a cell population” refers to a group of at least two cells. In non-limiting examples, a cell population can include at least about 10, at least about 100, at least about 200, at least about 300, at least about 400, at least about 500, at least about 600, at least about 700, at least about 800, at least about 900, at least about 1000 cells, at least about 5,000 cells or at least about 10,000 cells or at least about 100,000 cells or at least about 1,000,000 cells. The population may be a pure population comprising one cell type, such as a population of glial competent cells (e.g., astrocyte precursors), or a population of undifferentiated stem cells. Alternatively, the population may comprise more than one cell type, for example a mixed cell population.

As used herein, the term “stem cell” refers to a cell with the ability to divide for indefinite periods in culture and to give rise to specialized cells. A human stem cell refers to a stem cell that is from a human.

As used herein, the term “embryonic stem cell” refers to a primitive (undifferentiated) cell that is derived from preimplantation-stage embryo, capable of dividing without differentiating for a prolonged period in culture, and are known to develop into cells and tissues of the three primary germ layers. A human embryonic stem cell refers to an embryonic stem cell that is from a human. As used herein, the term “human embryonic stem cell” or “hESC” refers to a type of pluripotent stem cells derived from early stage human embryos, up to and including the blastocyst stage, that is capable of dividing without differentiating for a prolonged period in culture, and are known to develop into cells and tissues of the three primary germ layers.

As used herein, the term “embryonic stem cell line” refers to a population of embryonic stem cells which have been cultured under in vitro conditions that allow proliferation without differentiation for up to days, months to years. For example, “embryonic stem cell” can refers to a primitive (undifferentiated) cell that is derived from preimplantation-stage embryo, capable of dividing without differentiating for a prolonged period in culture, and are known to develop into cells and tissues of the three primary germ layers. A human embryonic stem cell refers to an embryonic stem cell that is from a human. As used herein, the term “human embryonic stem cell” or “hESC” refers to a type of pluripotent stem cells derived from early stage human embryos, up to and including the blastocyst stage, that is capable of dividing without differentiating for a prolonged period in culture, and are known to develop into cells and tissues of the three primary germ layers.

As used herein, the term “pluripotent” refers to an ability to develop into the three developmental germ layers of the organism including endoderm, mesoderm, and ectoderm.

As used herein, the term “induced pluripotent stem cell” or “iPSC” refers to a type of pluripotent stem cell, similar to an embryonic stem cell, formed by the introduction of certain embryonic genes (such as a OCT4, SOX2, and KLF4 transgenes) (see, for example, Takahashi and Yamanaka Cell 126, 663-676 (2006), herein incorporated by reference) into a somatic cell, for examples, CI 4, C72, and the like.

As used herein, the term “somatic cell” refers to any cell in the body other than gametes (egg or sperm); sometimes referred to as “adult” cells.

As used herein, the term “somatic (adult) stem cell” refers to a relatively rare undifferentiated cell found in many organs and differentiated tissues with a limited capacity for both self-renewal (in the laboratory) and differentiation. Such cells vary in their differentiation capacity, but it is usually limited to cell types in the organ of origin.

As used herein, the term “neuron” refers to a nerve cell, the principal functional units of the nervous system. A neuron consists of a cell body and its processes—an axon and one or more dendrites. Neurons transmit information to other neurons or cells by releasing neurotransmitters at synapses.

As used herein, the term “proliferation” refers to an increase in cell number.

As used herein, the term “undifferentiated” refers to a cell that has not yet developed into a specialized cell type.

As used herein, the term “differentiation” refers to a process whereby an unspecialized embryonic cell acquires the features of a specialized cell such as a heart, liver, or muscle cell.

Differentiation is controlled by the interaction of a cell's genes with the physical and chemical conditions outside the cell, usually through signaling pathways involving proteins embedded in the cell surface.

As used herein, the term “directed differentiation” refers to a manipulation of stem cell culture conditions to induce differentiation into a particular (for example, desired) cell type, such as astrocytes and precursors thereof. In certain embodiments, the term “directed differentiation” in reference to a stem cell refers to the use of small molecules, growth factor proteins, and other growth conditions to promote the transition of a stem cell from the pluripotent state into a more mature or specialized cell fate (e.g. astrocytes, etc.).

As used herein, the term “inducing differentiation” in reference to a cell refers to changing the default cell type (genotype and/or phenotype) to a non-default cell type (genotype and/or phenotype). Thus, “inducing differentiation in a stem cell” refers to inducing the stem cell (e.g., human stem cell) to divide into progeny cells with characteristics that are different from the stem cell, such as genotype (e.g., change in gene expression as determined by genetic analysis such as a microarray) and/or phenotype (e.g., change in expression of a protein, such as GFAP, AQP4, CD44, S100b, SOX9, GLT-1, CSRP1 and/or NFIA).

As used herein, the term “culture medium” refers to a liquid that covers cells in a culture vessel, such as a Petri plate, a multi-well plate, and the like, and contains nutrients to nourish and support the cells. Culture medium may also include growth factors added to produce desired changes in the cells.

As used herein, the term “contacting” cells with a compound (e.g., one or more inhibitor, activator, and/or inducer) refers to exposing cells to a compound, for example, placing the compound in a location that will allow it to touch the cell. The contacting may be accomplished using any suitable methods. For example, contacting can be accomplished by adding the compound to a tube of cells. Contacting may also be accomplished by adding the compound to a culture medium comprising the cells. Each of the compounds (e.g., the inhibitors, activators, and/or inducers) can be added to a culture medium comprising the cells as a solution (e.g., a concentrated solution). Alternatively or additionally, the compounds (e.g., the inhibitors, activators, and inducers disclosed herein) as well as the cells can be present in a formulated cell culture medium.

An effective amount is an amount that produces a desired effect.

As used herein, the term “in vitro” refers to an artificial environment and to processes or reactions that occur within an artificial environment. In vitro environments exemplified, but are not limited to, test tubes and cell cultures.

As used herein, the term “in vivo” refers to the natural environment (e.g., an animal or a cell) and to processes or reactions that occur within a natural environment, such as embryonic development, cell differentiation, neural tube formation, etc.

As used herein, the term “expressing” in relation to a gene or protein refers to making an mRNA or protein which can be observed using assays such as microarray assays, antibody staining assays, and the like.

As used herein, the term “marker” or “cell marker” refers to gene or protein that identifies a particular cell or cell type, e.g., astrocyte, or glial competent cell (e.g., astrocyte precursor). A marker for a cell may not be limited to one marker, markers may refer to a “pattern” of markers such that a designated group of markers may identity a cell or cell type from another cell or cell type.

As used herein, the term “derived from” or “established from” or “differentiated from” when made in reference to any cell disclosed herein refers to a cell that was obtained from (e.g., isolated, purified, etc.) a parent cell in a cell line, tissue (such as a dissociated embryo, or fluids using any manipulation, such as, without limitation, single cell isolation, cultured in vitro, treatment and/or mutagenesis using for example proteins, chemicals, radiation, infection with virus, transfection with DNA sequences, such as with a morphogen, etc., selection (such as by serial culture) of any cell that is contained in cultured parent cells. A derived cell can be selected from a mixed population by virtue of response to a growth factor, cytokine, selected progression of cytokine treatments, adhesiveness, lack of adhesiveness, sorting procedure, and the like.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

As used herein, the term “disease” refers to any condition or disorder that damages or interferes with the normal function of a cell, tissue, or organ.

As used herein, the term “treating” or “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

The term “differentiation day” as used herein, refers to a time line having twenty-four-hour intervals (i.e., days) after a stem cell culture is contacted by differentiation molecules. For example, such molecules may include, but are not limited to, SMAD inhibitor molecules and NFIA activators. The day of contacting the culture with the molecules is referred to as differentiation day 1. For example, differentiation day 2 represents anytime between twenty-four and forty-eight hours after the stem cell culture had been contacted by a differentiation molecule.

### 5.2 Methods of Differentiation Stem Cells

The presently disclosed subject matter provides in vitro methods for inducing differentiation of stem cells. In certain embodiments, the stem cells are human stem cells. Non-limiting examples of human stem cells include human embryonic stem cells (hESC), human pluripotent stem cell (hPSC), human induced pluripotent stem cells (hiPSC), human parthenogenetic stem cells, primordial germ cell-like pluripotent stem cells, epiblast stem cells, F-class pluripotent stem cells, somatic stem cells, cancer stem cells, or any other cell capable of lineage specific differentiation. In certain embodiments, the human stem cell is a human embryonic stem cell (hESC). In certain embodiments, the human stem cell is a human induced pluripotent stem cell (hiPSC). In certain embodiments, the stem cells are non-human stem cells. Non-limiting examples of non-human stem cells non-human primate stem cells, rodent stem cells, dog stem cells, cat stem cells. In certain embodiments, the stem cells are pluripotent stem cells. In certain embodiments, the stem cells are embryonic stem cells. In certain embodiments, the stem cells are induced pluripotent stem cells (iPSCs). In certain embodiments, the stem cells are non-human stem cells, including, but not limited to, mammalian stem cells, primate stem cells, or stem cells from a rodent, a mouse, a rat, a dog, a cat, a horse, a pig, a cow, a sheep, etc.

The presently disclosed subject matter provides stem-cell-derived glial competent cells (e.g., astrocyte precursors) and astrocytes. In certain embodiments, the differentiation of stem cells to astrocytes include three phases: (a) in vitro differentiation of stem cells to NSCs, (b) in vitro differentiation of NSCs to glial competent cells (e.g., astrocyte precursors), and (c) in vitro differentiation of glial competent cells to astrocytes. In certain embodiments, a population of stem cells are in vitro differentiated to a population of NSCs, which are in vitro differentiated to a population of glial competent cells (e.g., astrocyte precursors), which are further differentiated in vitro to a population of astrocytes. In certain embodiments, the NSCs are in vitro differentiated from stem cells by SMAD inhibition. In certain embodiments, the method comprises contacting a population of stem cells (e.g., human stem cells) with one or more SMAD inhibitor signaling (e.g., one or more inhibitor of TGFβ/Activin-Nodal signaling and/or one or more BMP inhibitor. In certain embodiments, the glial competent cells are astrocyte precursors.

In certain embodiments, the glial competent cells are in vitro differentiated from NSCs by inducing glial competency. In certain embodiments, inducing glial competency is achieved by promoting NFIA signaling in the cells (e.g., the NSCs derived from stem cells by inhibition of SMAD signaling). In certain embodiments, inducing glial competency is achieved by lengthening G1 phase of the cell cycle in the cells (e.g., the NSCs derived from stem cells by inhibition of SMAD signaling).

In certain embodiments, the astrocytes are in vitro differentiated from glial competent cells by accelerating astrocyte differentiation. In certain embodiments, astrocyte differentiation is achieved by decreasing the expression of NFIA in the cells (e.g., the glial competent cells derived from NSCs) (e.g., by exposing the cells to an inhibitor of NFIA signaling). In certain embodiments, astrocyte differentiation is achieved by exposing the cells (e.g., the glial competent cells derived from NSCs) to LIF, one or more derivative thereof, one or more analog thereof, and/or one or more activator thereof.

In certain embodiments, the differentiation of stems cells to astrocytes include two phases: (a) in vitro differentiation of stem cells to NSCs, and (b) in vitro differentiation of NSCs to astrocytes. In certain embodiments, a population of stem cells are in vitro differentiated to a population of NSCs, which are in vitro differentiated to a population of astrocytes. In certain embodiments, the astrocytes are in vitro differentiated from NSCs by inducing astrocyte differentiation. In certain embodiments, inducing astrocyte differentiation is achieved by exposing a population of NSCs (e.g., the NSCs derived from stem cells by inhibition of SMAD signaling) with a fetal bovine serum (FBS).

The presently disclosed subject matter is also directed to stem-cell-derived regional glial competent cells and regional astrocytes. In certain embodiments, the differentiation of stem cells to regional astrocytes include three phases: (a) in vitro differentiation of stem cells to regionally patterned progenitors, (b) in vitro differentiation of regionally patterned progenitors to regional glial competent cells, and (c) in vitro differentiation or maturation of regional glial competent cells to regional astrocytes. In certain embodiments, a population of stem cells are in vitro differentiated to a population of regionally pattered precursors, which are in vitro differentiated to a population of regional glial competent cells, which are in vitro differentiated to a population of regional astrocytes.

In certain embodiments, the regionally patterned progenitors are cortical progenitors, the regional glial competent cells are cortical glial competent cells, and the regional astrocytes are cortical astrocytes.

In certain embodiments, the regionally patterned progenitors are spinal cord progenitors, the regional glial competent cells are spinal cord glial competent cells, and the regional astrocytes are spinal cord astrocytes.

In certain embodiments, the cortical progenitors are in vitro differentiated from stem cells by inhibition of Wnt signaling (referred to as “Wnt inhibitor”). In certain embodiments, the method comprises exposing a population of stem cells (e.g., human stem cells) to one or more Wnt inhibitor.

In certain embodiments, the spinal cord progenitors are in vitro differentiated from stem cells by inducing glial competency. In certain embodiments, the inducing glial competency is achieved by exposing a population of stem cells (e.g., human stem cells) to one or more RA activator and/or one or more SHH activator.

In certain embodiments, the cortical glial competent cells are in vitro differentiated from cortical progenitors by promoting NFIA signaling in the cortical progenitors. In certain embodiments, the cortical glial competent cells are in vitro differentiated from cortical progenitors by lengthening G1 phase of the cell cycle in the cortical progenitors.

In certain embodiments, the cortical astrocytes are in vitro differentiated from cortical glial competent cells by accelerating astrocyte differentiation. In certain embodiments, the astrocyte differentiation is achieved by decreasing the expression of NFIA (or exposing the cells to one or more NFIA inhibitor) in the cortical glial competent cells. In certain embodiments, the astrocyte differentiation is achieved by exposing a population of cortical glial competent cells to LIF, one or more derivative thereof, one or more analog thereof, and/or or one or more activator thereof.

In certain embodiments, the spinal cord glial competent cells are in vitro differentiated from spinal cord progenitors by inducing glial competency. In certain embodiments, the inducing glial competency is achieved by promoting NFIA signaling in the spinal cord progenitors. In certain embodiments, the inducing glial competency is achieved by lengthening G1 phase of the cell cycle in the spinal cord progenitors.

In certain embodiments, the spinal cord astrocytes are in vitro differentiated from spinal cord glial competent cells by accelerating astrocyte differentiation. In certain embodiments, the accelerating astrocyte differentiation is achieved by decreasing the expression of NFIA (or contacting a NFIA inhibitor) in the spinal cord glial competent cells. In certain embodiments, the accelerating astrocyte differentiation is achieved by exposing the spinal cord glial competent cells to LIF, one or more derivative thereof, one or more analog thereof, and/or one or more activator thereof.

### 5.2.1. In Vitro Three-Phase Differentiation of Stem Cells to Astrocytes

In certain embodiments, the differentiation of stem cells to astrocytes include three phases: (a) in vitro differentiation of stem cells to NSCs, (b) in vitro differentiation of NSCs to glial competent cells, and (c) in vitro differentiation or maturation of glial competent cells to astrocytes. For example, stem cells are in vitro differentiated to cells expressing one or more NSC marker (including, but not limited to PAX6, NESTIN, SOX1, SOX2, PLZF, ZO-1, BRN2 (e.g., NSCs), which are in vitro differentiated to cells expressing one or more glial competent cell marker (e.g., glial competent cells, e.g., astrocyte precursors), which are further induced in vitro to cells expressing one or more astrocyte marker (e.g., astrocytes).

### 5.2.1.1. In Vitro Differentiation of Stem Cells to NSCs

In certain embodiments, the method of in vitro inducing differentiation of stem cells to NSCs (e.g., Rosette-stage neural stem cells, e.g., LT-NSC) comprises contacting a population of stem cells with one or more SMAD inhibitor. Non-limiting examples of SMAD inhibitors include inhibitors of TGFβ/Activin-Nodal signaling and BMP inhibitors.

In certain embodiments, a presently disclosed differentiation method comprises exposing a population of stem cells with one or more inhibitor of transforming growth factor beta (TGFβ)/Activin-Nodal signaling, which thereby inhibits SMAD signaling. In certain embodiments, the inhibitor of TGFβ/Activin-Nodal signaling neutralizes the ligands including TGFβs, BMPs, Nodal, and activins, or blocking their signal pathways through blocking the receptors and downstream effectors. Non-limiting examples of inhibitors of TGFβ/Activin-Nodal signaling are disclosed in WO/2010/096496, WO/2011/149762, WO/2013/067362, WO/2014/176606, WO/2015/077648, Chambers et al., Nature Biotechnology 27, 275-280 (2009), and Chambers et al., Nature biotechnology 30, 715-720 (2012), which are incorporated by reference in their entireties for all purposes. In certain embodiments, the one or more inhibitor of TGFβ/Activin-Nodal signaling is a small molecule selected from the group consisting of SB431542, derivatives thereof, and mixtures thereof. In certain embodiments, the one or more inhibitor of TGFβ/Activin-Nodal signaling comprises SB431542.

“SB431542” refers to a molecule with a number CAS 301836-41-9, a molecular formula of C22H18N4O3, and a name of 4-[4-(1,3-benzodioxol-5-yl)-5-(2-pyridinyl)-1H-imidazol-2-yl]-benzamide, for example, see structure below:

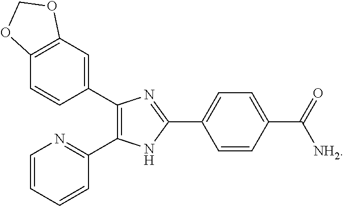 [SMILES: NC(=O)c1ccc(-c2nc(-c3ccc4c(c3)OCO4)c(-c3ccccn3)[nH]2)cc1]

In certain embodiments, the method of in vitro inducing differentiation of stem cells to NSCs (e.g., Rosette-stage neural stem cells, e.g., LTNSC) further comprises contacting the stem cells with one or more BMP inhibitor, which thereby inhibits SMAD signaling. Non-limiting examples of BMP inhibitors are disclosed in WO/2010/096496, WO/2011/149762, WO/2013/067362, WO/2014/176606, WO/2015/077648, Chambers et al., Nature Biotechnology 27, 275-280 (2009), and Chambers et al., Nature biotechnology 30, 715-720 (2012), which are incorporated by reference in their entireties for all purposes. In certain embodiments, the one or more BMP inhibitor is a small molecule selected from the group consisting of LDN193189, derivatives thereof, and mixtures thereof. In certain embodiments, the one or more BMP inhibitor comprises LDN193189.

“LDN193189” refers to a small molecule DM-3189, IUPAC name 4-(6-(4-(piperazin-1-yl)phenyl)pyrazolo[1,5-a]pyrimidin-3-yl)quinoline, with a chemical formula of C₂₅H₂₂N₆ with the following formula:

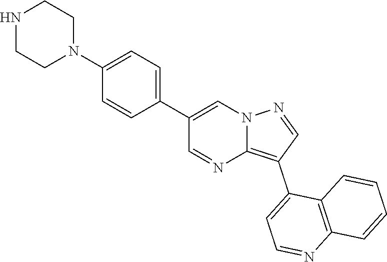 [SMILES: c1ccc2c(-c3cnn4cc(-c5ccc(N6CCNCC6)cc5)cnc34)ccnc2c1]

LDN193189 is capable of functioning as a SMAD signaling inhibitor. LDN193189 is also highly potent small-molecule inhibitor of ALK2, ALK3, and ALK6, protein tyrosine kinases (PTK), inhibiting signaling of members of the ALK1 and ALK3 families of type I TGFβ receptors, resulting in the inhibition of the transmission of multiple biological signals, including the bone morphogenetic proteins (BMP) BMP2, BMP4, BMP6, BMP7, and Activin cytokine signals and subsequently SMAD phosphorylation of Smadl, Smad5, and Smad8 (Yu et al. (2008) Nat Med 14:1363-1369; Cuny et al. (2008) Bioorg. Med. Chem. Lett. 18: 4388-4392, herein incorporated by reference).

For in vitro differentiation of stem cells to NSCs, the stem cells can be contacted with the one or more inhibitor of TGFβ/Activin-Nodal signaling for at least about 5 days, at least about 6 days, at least about 7 days, at least about 8 days, at least about 9 days, at least about 10 days, at least about 11 days, at least about 12 days, at least about 13 days, at least about 14 days, or at least about 15 days. In certain embodiments, the stem cells are contacted with the one or more inhibitor of TGFβ/Activin-Nodal signaling for up to about 5 days, for up to about 6 days, for up to about 7 days, for up to about 8 days, for up to about 9 days, for up to about 10 days, for up to about 11 days, for up to about 12 days, for up to about 13 days, for up to about 14 days, or for up to about 15 days. In certain embodiments, the stem cells are contacted with the one or more inhibitor of TGFβ/Activin-Nodal signaling for between about 5 days and about 15 days, between about 5 days and about 10 days, or between about 10 days and about 15 days. In certain embodiments, the stem cells are contacted with the one or more inhibitor of TGFβ/Activin-Nodal signaling for between about 10 days and about 15 days. In certain embodiments, the stem cells are contacted with the one or more inhibitor of TGFβ/Activin-Nodal signaling for about 5 days, about 6 days, about 7 days, about 8 days, about 9 days, about 10 days, about 11 days, about 12 days, about 13 days, about 14 days, or about 15 days. In certain embodiments, the stem cells are contacted with the one or more inhibitor of TGFβ/Activin-Nodal signaling for about 8 days. In certain embodiments, the stem cells are contacted with the one or more inhibitor of TGFβ/Activin-Nodal signaling for about 10 days. In certain embodiments, the stem cells are contacted with the one or more inhibitor of TGFβ/Activin-Nodal signaling for about 11 days. In certain embodiments, the stem cells are contacted with the one or more inhibitor of TGFβ/Activin-Nodal signaling for about 12 days.

For in vitro differentiation of stem cells to NSCs, the stem cells can be contacted with the one or more BMP inhibitor for at least about 5 days, at least about 6 days, at least about 7 days, at least about 8 days, at least about 9 days, at least about 10 days, at least about 11 days, at least about 12 days, at least about 13 days, at least about 14 days, or at least about 15 days. In certain embodiments, the stem cells are contacted with the one or more BMP inhibitor for up to about 5 days, for up to about 6 days, for up to about 7 days, for up to about 8 days, for up to about 9 days, for up to about 10 days, for up to about 11 days, for up to about 12 days, for up to about 13 days, for up to about 14 days, or for up to about 15 days. In certain embodiments, the stem cells are contacted with the one or more BMP inhibitor for between about 5 days and about 15 days, between about 5 days and about 10 days, or between about 10 days and about 15 days. In certain embodiments, the stem cells are contacted with the one or more BMP inhibitor for between about 10 days and about 15 days. In certain embodiments, the stem cells are contacted with the one or more BMP inhibitor for about 5 days, about 6 days, about 7 days, about 8 days, about 9 days, about 10 days, about 11 days, about 12 days, about 13 days, about 14 days, or about 15 days. In certain embodiments, the stem cells are contacted with the one or more BMP inhibitor for about 8 days. In certain embodiments, the stem cells are contacted with the one or more BMP inhibitor for about 10 days. In certain embodiments, the stem cells are contacted with the one or more BMP inhibitor for about 11 days. In certain embodiments, the stem cells are contacted with the one or more BMP inhibitor for about 12 days.

In certain embodiments, the stem cells are contacted with the one or more inhibitor of TGFβ/Activin-Nodal signaling in a concentration of from about 1 μM to about 100 μM, from about 1 μM to about 20 μM, from about 1 μM to about 15 μM, from about 1 μM to about 10 μM, from about 1 μM to about 5 μM, from about 5 μM to about 10 μM, from about 5 μM to about 15 μM, from about 15 μM to about 20 μM, from about 20 μM to about 30 μM, from about 30 μM to about 40 μM, from about 40 μM to about 50 μM, from about 50 μM to about 60 μM, from about 60 μM to about 70 μM, from about 70 μM to about 80 μM, from about 80 μM to about 90 μM, or from about 90 μM to about 100 μM. In certain embodiments, the stem cells are contacted with the one or more inhibitor of TGFβ/Activin-Nodal signaling in a concentration of from about from about 5 μM to about 15 μM. In certain embodiments, the stem cells are contacted with the one or more inhibitor of TGFβ/Activin-Nodal signaling in a concentration of about 10 μM. In certain embodiments, the stem cells are contacted with the one or more inhibitor of TGFβ/Activin-Nodal signaling in any one of the above-described concentrations daily, every other day or every two days. In certain embodiments, the stem cells are contacted with the one or more inhibitor of TGFβ/Activin-Nodal signaling in a concentration of about 10 μM daily.

In certain embodiments, the stem cells are contacted with the one or more BMP inhibitor in a concentration of from about 1 nM to about 300 nM, from about 5 nM to about 250 nM, from about 10 nM to about 200 nM, from about 10 nM to about 50 nM, from about 50 nM to about 150 nM, from about 80 nM to about 120 nM, from about 90 nM to about 110 nM, from about 50 nM to about 100 nM, from about 100 nM to about 150 nM, from about 150 nM to about 200 nM, from about 200 nM to about 250 nM, or from about 250 nM to about 300 nM. In certain embodiments, the stem cells are contacted with the one or more BMP inhibitor in a concentration of from about 80 nM to about 120 nM. In certain embodiments, the stem cells are contacted with the one or more BMP inhibitor in a concentration of about 100 nM. In certain embodiments, the stem cells are contacted with the one or more BMP inhibitor in any one of the above-described concentrations daily, every other day or every two days. In certain embodiments, the stem cells are contacted with the one or more BMP inhibitor in a concentration of about 100 nM daily.

In certain embodiments, the stem cells (e.g., human stem cells) are contacted with the one or more inhibitor of TGFβ/Activin-Nodal signaling and/or the one or more BMP inhibitor in effective amounts to produce a cell population comprising at least about 10% (e.g., at least about 20%, about 30%, about 40%, about 50%, about 60%, about 70%, about 80%, about 90%, about 95%, about 99% or more) cells expressing one or more neural stem cell (NSC) marker. Non-limiting examples of NSC markers include PAX6, NESTIN, SOX1, SOX2, PLZF, ZO-1, and BRN2. In certain embodiments, the one or more NSC marker is selected from the group consisting of PAX6, SOX1, PLZF, and ZO-1.

### 5.2.1.2. In Vitro Differentiation of NSCs to Glial Competent Cells

In certain embodiments, the method for inducing differentiation of NSCs to glial competent cells comprises promoting NFIA signaling in the NSCs (e.g., cells expressing one or more NSC marker, e.g., the differentiated cells obtained by the method described in Section 5.2.1.1) to produce a cell population comprising at least about 10% differentiated cells that express one or more glial competent cell marker.

In certain embodiments, the method for inducing differentiation of NSCs to glial competent cells comprises lengthening G1 phase of the cell cycle of the NSCs (e.g., cells expressing one or more NSC marker, e.g., the differentiated cells obtained by the method described in Section 5.2.1.1) to produce a cell population comprising at least about 10% differentiated cells that express one or more glial competent cell marker.

In certain embodiments, lengthening G1 phase of the cell cycle of the NSCs comprises exposing the NSCs to one or more G1 phase lengthening compound (e.g., Olomoucine). In certain embodiments, lengthening G1 phase of the cell cycle of the NSCs comprises increasing expression of FZR1 (also known as APC^{CDH1}).

In certain embodiments, the glial competent cells are astrocyte precursors.

In certain embodiments, the method further comprises contacting the cells with one or more activator of FGF signaling (“FGF activator”) and/or one or more EGF-family protein.

Non-limiting examples of FGF activators include FGF1, FGF2, FGF3, FGF4, FGF7, FGF8, FGF10, FGF18, derivatives, and mixtures thereof. In certain embodiments, the one or more FGF activator is FGF2.

Non-limiting examples of EGF-family protein include EGF, Heparin-binding EGF-like growth factor (HB-EGF), Epiregulin (EPR), Epigen, Betacellulin (BTC), neuregulin-1 (NRG1), neuregulin-2 (NRG2), neuregulin-3 (NRG3), neuregulin-4 (NRG4), and mixtures thereof. In certain embodiments, the one or more EGF-family protein is EGF.

### 5.2.1.2.1. Promoting NFIA Signaling

In certain embodiments, promoting NFIA signaling in the NSCs comprises exposing the NSCs to one or more NFIA activator. In certain embodiments, promoting NFIA signaling in the NSCs comprises increasing expression of NFIA.

In certain embodiments, the NFIA activator is an upstream activator of NFIA. In certain embodiments, the upstream activator of NFIA is TGFβ1.

In certain embodiments, increasing expression of NFIA comprises modifying the NSCs to induce overexpression of NFIA. In certain embodiments, the modified cells express a recombinant NFIA protein, for example, a NFIA nucleic acid (e.g., NFIA cDNA). In certain embodiments, expression of the NFIA nucleic acid is operably linked to an inducible promoter.

In certain embodiments, the NFIA nucleic acid is delivered into the cells using a retroviral vector, e.g., gamma-retroviral vectors, and lentiviral vectors. Combinations of retroviral vector and an appropriate packaging line are suitable, where the capsid proteins can be functional for infecting human cells. Non-limiting examples of amphotropic virus-producing cell lines include PA12 (Miller, et al. (1985) Mol. Cell. Biol. 5:431-437); PA317 (Miller, et al. (1986) Mol. Cell. Biol. 6:2895-2902); and CRIP (Danos, et al. (1988) Proc. Natl. Acad. Sci. USA 85:6460-6464). Non-amphotropic particles can also be used. Non-limiting examples of non-amphotropic particles include particles pseudotyped with VSVG, RD114 or GALV envelope and any other known in the art.

Non-limiting examples of transduction methods include cell culture with producer cells (e.g., by the method of Bregni, et al. (1992) Blood 80:1418-1422), cell culture with viral supernatant alone or concentrated vector stocks with or without appropriate growth factors and polycations (e.g., by the method of Xu, et al. (1994) Exp. Hemat. 22:223-230; and Hughes, et al. (1992) J. Clin. Invest. 89:1817). Alternatively or additionally, transducing viral vectors can be used to deliver the NFIA nucleic acid to the cells. In certain embodiments, the vector exhibits high efficiency of infection and stable integration and expression. Non-limiting examples of viral vectors include retroviral vectors, adenoviral vectors, lentiviral vectors, and adena-associated viral vectors, vaccinia virus, a bovine papilloma virus, or a herpes virus (e.g., Epstein-Barr Virus).

Non-viral approaches can also be employed for delivering the NFIA nucleic acid to the cells. For example, a nucleic acid molecule can be introduced into the NSCs by administering the nucleic acid in the presence of lipofection, asialoorosomucoid-polylysine conjugation, or by micro-injection under surgical conditions. Other non-viral means for gene transfer include transfection in vitro using calcium phosphate, DEAE dextran, electroporation, and protoplast fusion. Liposomes can also be potentially beneficial for delivery of nucleic acid molecules into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically.

In certain embodiments, the promotion of NFIA signaling is concurrent with the exposure of the cells to the one or more FGF activator and/or one or more EGF-family protein. In certain embodiments, the one or more FGF activator and/or the one or more EGF-family protein are both present in a cell culture medium comprising the cells whose NFIA signaling has been or is being promoted. In certain embodiments, the promotion of NFIA signaling is exposing the cells to one or more NFIA activator (e.g., TGFβ1). In certain embodiments, the one or more NFIA activator, the one or more FGF activator and the one or more EGF-family protein are added together daily (or every other day or every two days) to a cell culture medium comprising the NSCs.

In certain embodiments, the NFIA signaling is promoted in the NSCs (and optionally the NSCs are exposed to an effective amount of the one or more FGF activator and/or an effective amount of the one of more EGF-family protein) for at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days or more; or for up to about 2 days, for up to about 3 days, for up to about 4 days, for up to about 5 days, for up to about 6 days, for up to about 7 days, for up to about 8 days, for up to about 9 days, for up to about 10 days, for up to about 11 days, for up to about 12 days, for up to about 13 days, for up to about 14 days, for up to about 15 days, for up to about 16 days, for up to about 17 days, for up to about 18 days, for up to about 19 days, for up to about 20 days, to produce glial competent cells. In certain embodiments, the NFIA signaling is promoted in the NSCs and optionally the NSCs are contacted with an effective amount of the one or more FGF activator and/or an effective amount of the one of more EGF-family protein) for about 2, 3, 4, 5, 6, 7, 8, 9, or 10 days. In certain embodiments, the NFIA signaling is promoted in the NSCs and optionally the NSCs are contacted with an effective amount of the one or more FGF activator and/or an effective amount of the one of more EGF-family protein) for about 5 days.

In certain embodiments, the promotion of NFIA signaling is exposing the cells to one or more NFIA activator. In certain embodiments, the NFIA activator is an upstream activator of NFIA. In certain embodiments, the upstream activator of NFIA is TGFβ1.

In certain embodiments, the NSCs are contacted with the one or more NFIA activator in a concentration of from about 1 ng/ml to 100 ng/ml, from about 1 ng/ml to 20 ng/ml, from about 1 ng/ml to 15 ng/ml, from about 1 ng/ml to 10 ng/ml, from about 1 ng/ml to 5 ng/ml, from about 5 ng/ml to 10 ng/ml, from about 5 ng/ml to 15 ng/ml, from about 15 ng/ml to 25 ng/ml, from about 15 ng/ml to 20 ng/ml, from about 20 ng/ml to 30 ng/ml, from about 30 ng/ml to 40 ng/ml, from about 40 ng/ml to 50 ng/ml, from about 50 ng/ml to 60 ng/ml, from about 60 ng/ml to 70 ng/ml, from about 70 ng/ml to 80 ng/ml, from about 80 ng/ml to 90 ng/ml, or from about 90 ng/ml to 100 ng/ml to produce glial competent NSCs. In certain embodiments, the cells (e.g., NSCs) are contacted with the one or more NFIA activator (e.g., TGFβ1) in a concentration of from about 5 ng/ml to 15 ng/ml to produce glial competent NSCs. In certain embodiments, the cells (e.g., NSCs) are contacted with the one or more TGFβ1 in a concentration of about 10 ng/ml to produce glial competent NSCs. In certain embodiments, the cells (e.g., NSCs) are contacted with the one or more TGFβ1 in any one of the above-described concentrations daily, every other day or every two days to produce glial competent NSCs. In certain embodiments, the cells (e.g., NSCs) are contacted with the one or more NFIA activator (e.g., TGFβ1) in a concentration of about 10 ng/ml daily to produce glial competent NSCs. In certain embodiments, the cells (e.g., NSCs) are contacted with the one or more NFIA activator (e.g., TGFβ1) in a concentration of about 10 ng/ml daily to produce glial competent NSCs.

In certain embodiments, the NSCs are contacted with the one or more NFIA activator (e.g., TGFβ1) for at least about 5 days, least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days or more; and/or for up to about 5 days, for up to about 6 days, for up to about 7 days, for up to about 8 days, for up to about 9 days, for up to about 10 days, for up to about 11 days, for up to about 12 days, for up to about 13 days, for up to about 14 days, for up to about 15 days, for up to about 16 days, for up to about 17 days, for up to about 18 days, for up to about 19 days, for up to about 20 days, for up to about 21 days, for up to about 22 days, for up to about 23 days, for up to about 24 days, for up to about 25 days, for up to about 26 days, for up to about 27 days, for up to about 28 days, for up to about 29 days, or for up to about 30 days, to produce glial competent cells. In certain embodiments, the NSCs are contacted with an effective amount of the one or more NFIA activator (e.g., TGFβ1) for between about 5 days and about 15 days, or between about 10 days and about 20 days, to produce the glial competent cells. In certain embodiments, the NSCs are contacted with an effective amount of the one or more NFIA activator (e.g., TGFβ1) for about 5 days (e.g., about 7 days) to produce the glial competent NSCs. In certain embodiments, the NSCs are contacted with an effective amount of the one or more TGFβ1 for about 15 (e.g., about 14) days to produce the glial competent NSCs.

In certain embodiments, the cells (e.g., NSCs) are contacted with the one or more FGF activator in a concentration of from about 1 ng/ml to 100 ng/ml, from about 1 ng/ml to 20 ng/ml, from about 1 ng/ml to 15 ng/ml, from about 1 ng/ml to 10 ng/ml, from about 1 ng/ml to 5 ng/ml, from about 5 ng/ml to 10 ng/ml, from about 5 ng/ml to 15 ng/ml, from about 15 ng/ml to 25 ng/ml, from about 15 ng/ml to 20 ng/ml, from about 20 ng/ml to 30 ng/ml, from about 30 ng/ml to 40 ng/ml, from about 40 ng/ml to 50 ng/ml, from about 50 ng/ml to 60 ng/ml, from about 60 ng/ml to 70 ng/ml, from about 70 ng/ml to 80 ng/ml, from about 80 ng/ml to 90 ng/ml, or from about 90 ng/ml to 100 ng/ml to produce glial competent cells. In certain embodiments, the cells (e.g., NSCs) are contacted with the one or more FGF activator in a concentration of from about from about 5 ng/ml to 15 ng/ml to produce glial competent cells. In certain embodiments, the cells (e.g., NSCs) are contacted with the one or more FGF activator in a concentration of about 10 ng/ml to produce glial competent cells. In certain embodiments, the cells (e.g., NSCs) are contacted with the one or more FGF activator in any one of the above-described concentrations daily, every other day or every two days to produce glial competent cells. In certain embodiments, the cells (e.g., NSCs) are contacted with the one or more FGF activator in a concentration of about 10 ng/ml daily to produce glial competent cells. In certain embodiments, the cells (e.g., NSCs) are contacted with the one or more FGF activator in a concentration of about 10 ng/ml daily to produce glial competent cells.

In certain embodiments, the cells (e.g., NSCs) are contacted with the one or more EGF-family protein in a concentration of from about 1 ng/ml to 100 ng/ml, from about 1 ng/ml to 20 ng/ml, from about 1 ng/ml to 15 ng/ml, from about 1 ng/ml to 10 ng/ml, from about 1 ng/ml to 5 ng/ml, from about 5 ng/ml to 10 ng/ml, from about 5 ng/ml to 15 ng/ml, from about 15 ng/ml to 25 ng/ml, from about 15 ng/ml to 20 ng/ml, from about 20 ng/ml to 30 ng/ml, from about 30 ng/ml to 40 ng/ml, from about 40 ng/ml to 50 ng/ml, from about 50 ng/ml to 60 ng/ml, from about 60 ng/ml to 70 ng/ml, from about 70 ng/ml to 80 ng/ml, from about 80 ng/ml to 90 ng/ml, or from about 90 ng/ml to 100 ng/ml to produce glial competent cells. In certain embodiments, the cells (e.g., NSCs) are contacted with the one or more EGF-family protein in a concentration of from about from about 5 ng/ml to 15 ng/ml to produce glial competent cells. In certain embodiments, the cells (e.g., NSCs) are contacted with the one or more EGF-family protein in a concentration of about 10 ng/ml to produce glial competent cells. In certain embodiments, the cells (e.g., NSCs) are contacted with the one or more EGF-family protein in any one of the above-described concentrations daily, every other day or every two days to produce glial competent cells. In certain embodiments, the cells (e.g., NSCs) are contacted with the one or more EGF-family protein in a concentration of about 10 ng/ml daily to produce glial competent cells. In certain embodiments, the cells (e.g., NSCs) are contacted with the one or more EGF-family protein in a concentration of about 10 ng/ml daily to produce glial competent cells.

In certain embodiments, a cell population comprising at least about 10% (e.g., about 50%) NSCs (e.g., cells expressing one or more NSC marker) are differentiated into a cell population comprising at least about 10% cells expressing one or more glial competent cell marker by promoting the NFIA signaling in the cells, and exposing the cells to one FGF activator (e.g., FGF2, e.g., 10 ng/mL FGF2), and one EGF-family protein (e.g., EGF, e.g., 10 ng/mL EGF). In certain embodiments, a cell population comprising at least about 10% (e.g., about 50%) NSCs (e.g., cells expressing one or more NSC marker) are differentiated into a cell population comprising at least about 10% cells expressing one or more glial competent cell marker by exposing the cells to one or more NFIA activator (e.g., TFGβ1, e.g., 10 ng/mL TFGβ1) for between about 5 and about 20 days, one FGF activator (e.g., FGF2, e.g., 10 ng/mL FGF2), and one EGF-family protein (e.g., EGF, e.g., 10 ng/mL EGF).

In certain embodiments, the promotion of NFIA signaling leads to an increase in the detectable level of one or more glial competent cell marker (including, but not limited to, CD44 AQP4, SOX2, and/or NECTIN) in a plurality of the cells. In certain embodiments, at least about 10%, about 20%, about 30%, about 40%, about 50%, about 60%, about 70%, about 80%, about 90%, about 95%, about 99% or more) of the cells express a detectable level of CD44 AQP4, SOX2, and/or NECTIN. In certain embodiments, at least about 99% or more of the cells express a detectable level of CD44 and at least about 10% express a detectable level of AQP4.

In certain embodiments, the differentiated cells expressing one or more glial competent cell marker do not express a detectable level of one or more neuronal marker (for example, Tuj1, MAP2, and DCX).

In certain embodiments, the exposure of the NSCs to the one or more NFIA activator is discontinued or decreased for a period of time effective to increase a detectable level of expression of one or more astrocyte marker (including, but not limited to, GFAP, AQP4, CD44, S100b, SOX9, GLT-1, CSRP1, and NFIA) in a plurality of the cells. In certain embodiments, at least about 10%, 20%, 30%, 40%, 50%, 60%, 70%, 80%, 90%, 95%, 99% or more of the cells express a detectable level of the one or more astrocyte marker. In certain embodiments, at least about 50% or more of the cells express a detectable level of the one or more astrocyte marker. In certain embodiments, the exposure of the NSCs to the one or more NFIA activator is discontinued or decreased for a period of time effective to decrease a detectable level of expression of SOX2, NESTIN, or both in a plurality of the cells. In certain embodiments, at least about 10%, 20%, 30%, 40%, 50%, 60%, 70%, 80%, 90%, 95%, 99% or more of the cells do not express a detectable level of SOX2, NESTIN, or both. In certain embodiments, at least about 50% or more of the cells do not express a detectable level of SOX2, NESTIN, or both.

In certain embodiments, said effective period of time is at least about 1 day, at least about 2 days, at least about 3 days, at least about 4 days, at least about 5 days, at least about 6 days, at least about 7 days, at least about 8 days, at least about 9 days, at least about 10 days; or for up to about 1 day, for up to about 2 days, for up to about 3 days, for up to about 4 days, for up to about 5 days, for up to about 6 days, for up to about 7 days, for up to about 8 days, for up to about 9 days, or for up to about 10 days. In certain embodiments, the effective period of time is about 5 days.

In certain embodiments, the expression level (or functional activity) of NFIA is decreased by about 10%, 20%, 30%, 40%, 50%, 60%, 70%, 75%, 80%, 85%, 90%, 95%, 98%, 99%, or 100% after contacting the cells with the one or more NFIA activator for at least, or up to about 2, 3, 4, 5, 6, 7, 8, 9, 10, 11, 12, 13, 14, 15, 16, 17, 18, 19, 20 or more days.

### 5.2.1.2.2. Lengthening G1 Phase of the Cell Cycle

In certain embodiments, the method for inducing differentiation of NSCs to glial competent cells comprises lengthening the G1 phase of the cell cycle of the NSCs. In certain embodiments, lengthening G1 phase of the cell cycle of the NSCs comprises exposing the NSCs to one or more G1 phase lengthening compound. In certain embodiments, the one or more G1 phase lengthening compound is a small molecule compound. In certain embodiments, the small molecule compound is Olomoucine (Olo), which is known to lengthen G1 timing in vitrO³⁶.

In certain embodiments, lengthening G1 phase of the cell cycle of the NSCs comprises increasing expression of FZR1 (also known as APC^{CDH1}). In certain embodiments, increasing expression of FZR1 comprises modifying the NSCs to induce overexpression of FZR1. In certain embodiments, the modified cells express a recombinant FZR1 protein, for example, an FZR1 nucleic acid (e.g., FZR1 cDNA) wherein expression of said FZR1 nucleic acid is operably linked to an inducible promoter. The FZR1 nucleic acid can be delivered into the NSCs using any methods known in the art and methods disclosed in Section 5.2.1.2.1.

In certain embodiments, the one or more G1 phase lengthening compound, one or more FGF activator and/or one or more EGF-family protein are all present in a cell culture medium comprising the NSCs. In certain embodiments, the one or more G1 phase lengthening compound, the one or more FGF activator and the one or more EGF-family protein are added together daily (or every other day or every two days) to a cell culture medium comprising the NSCs (e.g., cells expressing one or more NSC marker, e.g., differentiated cells after contacting a population of stem cells with one or more TGFβ/Activin-Nodal signaling and optionally one or more SMAD inhibitor).

In certain embodiments, the NSCs are further contacted with one or more FGF activator and/or one or more EGF-family protein. In certain embodiments, the NSCs are contacted with the one or more FGF activator and/or the one of more EGF-family protein for at least about 10 days, at least about 11 days, at least about 12 days, at least about 13 days, at least about 14 days, at least about 15 days, at least about 16 days, at least about 17 days, at least about 18 days, at least about 19 days, at least about 20 days or more; or for up to about 12 days, for up to about 13 days, for up to about 14 days, for up to about 15 days, for up to about 16 days, for up to about 17 days, for up to about 18 days, for up to about 19 days, for up to about 20 days or more, to produce glial competent cells. In certain embodiments, the NSCs are contacted with the one or more FGF activator and/or the one of more EGF-family protein for about 12, 13, 14, 15, 16, 17, 18, 19, or 20 days. In certain embodiments, the NSCs are contacted with the one or more FGF activator and/or the one of more EGF-family protein for between about 10 days and about 20 days, e.g., between about 10 days and about 15 days. In certain embodiments, the NSCs are contacted with the one or more FGF activator and/or the one of more EGF-family protein for about 12 days. In certain embodiments, the NSCs are contacted with the one or more FGF activator and/or the one of more EGF-family protein) for about 15 days. In certain embodiments, the cells (e.g., NSCs) are contacted with an effective amount of the one or more G1 phase lengthening compound (e.g., Olo) in a concentration of from about 10 uM to about 500 μM, from about 10 μM to about 400 μM, from about 10 μM to about 300 μM, from about 10 μM to about 200 μM, from about 20 μM to about 500 μM, from about 20 μM to about 400 μM, from about 20 μM to about 300 μM, from about 20 μM to about 200 μM, from about 30 μM to about 500 μM, from about 30 μM to about 400 μM, from about 30 μM to about 300 μM, from about 30 μM to about 200 μM, from about 40 μM to about 500 μM, from about 40 μM to about 400 μM, from about 40 μM to about 300 μM, from about 40 μM to about 200 μM, from about 50 μM to about 500 μM, from about 50 μM to about 400 μM, from about 50 μM to about 300 μM, from about 50 μM to about 200 μM to produce glial competent cells. In certain embodiments, the cells (e.g., NSCs) are contacted with an effective amount of the one or more G1 phase lengthening compound (e.g., Olo) in a concentration of from about 110 μM to about 150 μM to produce glial competent NSCs. In certain embodiments, the cells (e.g., NSCs) are contacted with an effective amount of the one or more G1 phase lengthening compound (e.g., Olo) in a concentration of about 100 μM to produce glial competent cells. In certain embodiments, the one or more G1 phase lengthening compound is Olomoucine.

In certain embodiments, the cells (e.g., NSCs) are contacted with an effective amount of the one or more G1 phase lengthening compound (e.g., Olo) for no more than about 2 days or 48 days, e.g., no more than about 18 hours, no more than about 24 hours, no more than about 36 hours to produce glial competent cells. In certain embodiments, the cells (e.g., NSCs) are contacted with an effective amount of the one or more G1 phase lengthening compound in any one of the above-described concentrations daily, every other day or every two days to produce glial competent cells. In certain embodiments, the cells (e.g., NSCs) are contacted with an effective amount of the one or more G1 phase lengthening compound in a concentration of about 100 μM daily to produce glial competent cells.

Any of the FGF activators and the EGF-family proteins disclosed in Section 5.2.1.2.1 can be used herein.

In certain embodiments, a cell population comprising at least about 10% (e.g., about 50%) NSCs (e.g., cells expressing one or more NSC marker) are differentiated into a cell population comprising at least about 10% (e.g., about 50%) cells expressing one or more glial competent cell marker by lengthening the G1 phase of the cell cycle of the NSCs, and exposing the cells to one FGF activator (e.g., FGF2, e.g., 10 nM FGF2), and one EGF-family protein (e.g., EGF, e.g., 10 nM EGF) for about 12 days. In certain embodiments, a cell population comprising at least about 10% (e.g., about 50%) NSCs (e.g., cells expressing one or more NSC marker) are differentiated into a cell population comprising at least about 10% (e.g., about 50%) cells expressing one glial competent cell marker by exposing the cells to one or more G1 phase lengthening molecule (e.g., Olomoucine, e.g., 100 μM Olomoucine) for no more than about 2 days, and exposing the cells to one FGF activator (e.g., FGF2, e.g., 10 nM FGF2), and one EGF-family protein (e.g., EGF, e.g., 10 nM EGF) for about 12 days.

### 5.2.1.3. In Vitro Differentiation of Glial Competent NSCs to Astrocytes

Glial competent cells can be further differentiated in vitro to astrocytes. The glial competent glial competent cells can be subjected to conditions favoring differentiation of glial competent cells into astrocytes.

In certain embodiments, the method comprises discontinuing, withdrawing, inhibiting and/or decreasing the exposure of the cells to the one or more NIFA activator (e.g., by contacting with an NFIA inhibitor, or decrease in NFIA expression level) after exposing the cells to the one or more NFIA activator for about 2, 3, 4, 5, 6, 7, 8, 9, 10, 11, 12, 13, 14, 15, 16, 17, 18, 19, 20 or more days.

In certain embodiments, contacting with the one or more NFIA activator is discontinued or decreased following an about 5-day or an about 8 day-exposure period. In certain embodiments, the conditions favoring differentiation of glial competent cells into astrocytes comprises exposing the cells to an effective amount of LIF (one or more derivative, analog and/or activator thereof) to increase the detectable level of the one or more astrocyte marker.

In certain embodiments, the cells are contacted to LIF for at least about 1, 2, 3, 4, 5, 6, 7, 8, 9, 10, 11, 12, 13, 14, 15 days or more; or for up to about 1, 2, 3, 4, 5, 6, 7, 8, 9, 10, 11, 12, 13, 14, 15 or more days. In certain embodiments, the cells are exposed to LIF for about 7, 8, 9, or 10 days.

In certain embodiments, the cells are exposed to LIF after or concurrently with the exposure of the cells to the one or more NFIA activator. In certain embodiments, the initial exposure of the cells to LIF is about 1, 2, 3, 4, or 5 days from the initial exposure of the cells to the one or more NFIA activator.

In certain embodiments, the in vitro method for inducing differentiation of stem cells into astrocytes and precursors thereof comprises exposing a population of stem cells with effective amounts of (i) one or more inhibitor of transforming growth factor beta (TGFβ)/Activin-Nodal signaling and/or one or more inhibitor of bone morphogenetic protein (BMP) signaling, (ii) one or more NFIA activator, and (iii) LIF (one or more derivative, analog and/or activator thereof). In certain embodiments, the initial exposure of the cells to the one or more NFIA activator is at least about 8 days from the initial exposure of the cells to the one or more inhibitor of TGFβ/Activin-Nodal signaling and/or one or more inhibitor of BMP signaling. In certain embodiments, the cells are exposed to the one or more NFIA activator for up to about 8 days. In certain embodiments, the initial exposure of the cells to LIF (one or more derivative, analog and/or activator thereof) at least about 2 days or at least about 5 days from the initial exposure of the cells to the one or more NFIA activator.

In certain embodiments, the day whereby the stem cells are contacted with the one or more inhibitor of TGFβ/Activin-Nodal signaling, and/or one or more inhibitor of BMP signaling corresponds to day 0.

In certain embodiments, the method further comprises subjecting said population of differentiated cells to conditions favoring maturation of said cells into a population of astrocytes. In certain embodiments, the conditions favoring maturation comprises culturing the cells in a suitable cell culture medium. In certain embodiments, the suitable cell culture medium comprises a neurobasal (NB) or N2 medium. In certain embodiments, the suitable cell culture medium is an NB medium supplemented with L-Glutamine, and B27 (e.g., from Life Technologies).

N2 supplement is a chemically defined, animal-free, supplement used for expansion of undifferentiated neural stem and progenitor cells in culture. N2 Supplement is intended for use with DMEM/F12 medium. The components of a N2 medium are disclosed in WO2011/149762. In certain embodiments, a N2 medium comprises a DMEM/F12 medium supplemented with glucose, sodium bicarbonate, putrescine, progesterone, sodium selenite, transferrin, and insulin. In certain embodiments, 1 liter of a N2 medium comprises 985 ml dist. H₂O with DMEM/F12 powder, 1.55 g of glucose, 2.00 g of sodium bicarbonate, putrescine (100 uL aliquot of 1.61 g dissolved in 100 mL of distilled water), progesterone (20 uL aliquot of 0.032 g dissolved in 100 mL 100% ethanol), sodium selenite (60 uL aliquot of 0.5 mM solution in distilled water), 100 mg of transferrin, and 25 mg of insulin in 10 mL of 5 mM NaOH.

### 5.2.2 Methods of In Vitro Differentiation of Stem Cells to Astrocytes Using FBS

In certain embodiments, the differentiation of stems cells to astrocytes include two phases: (a) in vitro differentiation of stem cells to NSCs, and (b) in vitro differentiation of NSCs to astrocytes. In certain embodiments, a population of stem cells are in vitro differentiated to a population of NSCs, which are in vitro differentiated to a population of astrocytes. In certain embodiments, the astrocytes are in vitro differentiated from NSCs by inducing astrocyte differentiation. In certain embodiments, inducing astrocyte differentiation is achieved by exposing a population of NSCs (e.g., the NSCs derived from stem cells by inhibition of SMAD signaling) with a fetal bovine serum (FBS).

In certain embodiments, the stem cells are differentiated to NSCs using the methods disclosed in Section 5.2.1.1 herein.

In certain embodiments, the method for inducing differentiation of NSCs to astrocytes comprises exposing the cells to an effective amount of fetal bovine serum (FBS). In certain embodiments, the FBS is in a composition exposed to the cells at a concentration of at least about 0.1%, 0.5%, 1%, 2%, 3%, 4%, 5%, 6%, 7%, 8%, 9%, or 10% FBS. In certain embodiments, the cells are exposed to the FBS for a period of time sufficient to increase a detectable level of expression of one or more astrocyte marker in a plurality of the cells. Non-limiting examples of astrocyte markers include GFAP, AQP4, CD44, S100b, SOX9, NFIA, GLT-1, and CSRP1. In certain embodiments, at least about 10%, 20%, 30%, 40%, 50%, 60%, 70%, 80%, 90%, 95%, 99% or more of the cells express a detectable level of one or more astrocyte marker (e.g., GFAP, AQP4, CD44, S100b, SOX9 GLT-1, CSRP1, and/or NFIA). In certain embodiments, the cells are exposed to the FBS for a period of time sufficient to decrease a detectable level of expression of SOX2, NESTIN, or both in a plurality of the cells. In certain embodiments, at least about 10%, 20%, 30%, 40%, 50%, 60%, 70%, 80%, 90%, 95%, 99% or more of the cells do not express a detectable level of SOX2, NESTIN, or both. In certain embodiments, at least about 50% or more of the cells do not express a detectable level of SOX2, NESTIN, or both.

In certain embodiments, the cells are exposed to a composition comprising FBS that does not comprise EGF and/or FGF2.

In certain embodiments, said sufficient/effective period of time is at least, or for up to, about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 or more days.

### 5.3 In Vitro Differentiation of Stem Cells to Regional Astrocytes

The presently disclosed subject matter also provides methods for differentiation of stem cells to regional astrocytes. In certain embodiments, the differentiation of stem cells to regional astrocytes include three phases: (a) in vitro differentiation of stem cells to regionally patterned progenitors, (b) in vitro differentiation of regionally patterned progenitors to regional glial competent cells, and (c) in vitro differentiation or maturation of regional glial competent cells to regional astrocytes. In certain embodiments, stem cells are in vitro differentiated to regionally pattered precursors, which are in vitro differentiated to regional glial competent cells, which are further differentiated in vitro to regional astrocytes. In certain embodiments, the regionally patterned progenitors are cortical progenitors, the regional glial competent cells are cortical glial competent cells, and the regional astrocytes are cortical astrocytes. In certain embodiments, the regionally patterned progenitors are spinal cord progenitors, the regional glial competent cells are spinal cord glial competent cells, and the regional astrocytes are spinal cord astrocytes.

### 5.3.1. In Vitro Differentiation of Stem Cells to Cortical Astrocytes

In certain embodiments, the methods of in vitro differentiation of stem cells to cortical progenitors comprise contacting the stem cells with one or more SMAD inhibitor (e.g., one or more inhibitor of transforming growth factor beta (TGFβ)/Activin-Nodal signaling, and/or one or more BMP inhibitor), and contacting the cells with one or more inhibitor of Wnt signaling (referred to as “Wnt inhibitor”) to obtain a cell population comprising at least about 10% cells expressing one or more cortical progenitor marker. Non-limiting examples of cortical progenitor markers include FOXG1, SOX2, NESTIN, and TBR2.

Any SMAD inhibitors disclosed in Section 5.2.1.1. can be used in these methods.

Non-limiting examples of Wnt inhibitors include XAV939, tankyrase inhibitors (e.g., those disclosed in Huang et al. Nature 461, 614-620 (2009)), Dickkopf (Dkk) proteins, secreted Frizzled-Related Proteins (sFRPs), IWR (e.g., those disclosed in Chen et al. Nature Chemical Biology 5(2): 100-107 (2009); and Kulak et al., Molecular and Cellular Biology, 4; 35(14):2425-35 (2015)), 2,4-diamino-quinazoline (e.g., disclosed in Chen et al., Bioorganic & medicinal chemistry letters, 1; 19(17):4980-3 (2009)), IWP (e.g., disclosed in Chen et al., Nat Chem Biol. 2009 February; 5(2):100-7), LGK974, C59 (e.g., disclosed in Proffitt et al., Cancer Res. 2013 Jan. 15; 73(2):502-7), Ant1.4Br/Ant 1.4Cl (e.g., disclosed in Morrell et al., PLoS One. 2008 Aug. 13; 3(8):e2930), Niclosamide (e.g., disclosed in Chen et al., Biochemistry. 2009 Nov. 3; 48(43): 10267-74), apicularen and bafilomycin (e.g., disclosed in Cruciat et al., Science. 2010 Jan. 22; 327(5964):459-63), G007-LK and G244-LM (e.g., disclosed in Lau et al., Cancer Res. 2013 May 15; 73(10):3132-44), pyrvinium (e.g., disclosed in Thorne et al., Nat Chem Biol. 2010 November; 6(11):829-36), NSC668036 (e.g., disclosed in Shan et al., Biochemistry. 2005 Nov. 29; 44(47):15495-503), Quercetin (e.g., disclosed in Park et al., Biochem Biophys Res Commun. 2005 Mar. 4; 328(1):227-34), ICG-001 (e.g., disclosed in Emami et al., Proc Natl Acad Sci USA. 2004 Aug. 24; 101(34):12682-7), PKF115-584 (e.g., disclosed in Lepourcelet et al., Cancer Cell. 2004 January; 5(1):91-102), BC2059 (e.g., disclosed in Fiskus et al., Leukemia. 2015 June; 29(6):1267-78), Shizokaol D (e.g., disclosed in Tang et al., PLoS One. 2016 Mar. 24; 11(3):e0152012), Wnt signaling inhibitors described in International Patent Publication No. WO2017/132596, filed Jan. 27, 2017, Wnt inhibitors disclosed in International Publication No. WO/2014/176606, filed Apr. 28, 2014, which are incorporated by reference in their entireties for all purposes, derivatives thereof, and mixtures thereof. In certain embodiments, the one or more Wnt inhibitor comprises XAV939.

In certain embodiments, XAV939 is 3,5,7,8-Tetrahydro-2-[4-(trifluoromethyl)phenyl]-4H-thiopyrano[4,3-d]pyrimidin-4-one, having the chemical formula C14H11F3N2OS. In certain embodiments, XAV939 has the following structure:

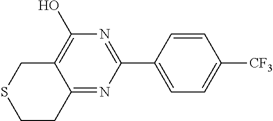 [SMILES: Oc1nc(-c2ccc(C(F)(F)F)cc2)nc2c1CSCC2]

The methods of differentiation of stem cells to cortical progenitors can be any methods disclosed in WO2017/132596, which is incorporated by reference in its entirety.

In certain embodiments, the cells are contacted with the one or more SMAD inhibitor (e.g., TGFβ)/Activin-Nodal inhibitor, and BMP inhibitor) and the one or more Wnt inhibitor for at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20 or more days to obtain a cell population comprising at least about 10% cells expressing one or more cortical progenitor marker. In certain embodiments, the cells are contacted with the one or more SMAD inhibitor (e.g., TGFβ)/Activin-Nodal inhibitor, and BMP inhibitor) and the one or more Wnt inhibitor for and/or up to about 4, up to about 5, up to about 6, up to about 7, up to about 8, up to about 9, up to about 10, up to about 11, up to about 12, up to about 13, up to about 14, up to about 15, up to about 16, up to about 17, up to about 18, up to about 19, up to about 20 or more days to obtain a cell population comprising at least about 10% cells expressing one or more cortical progenitor marker. In certain embodiments, the cells are contacted with one or more SMAD inhibitor (e.g., TGFβ)/Activin-Nodal inhibitor, and BMP inhibitor) and the one or more Wnt inhibitor for about 4, 5, 6, 7, 8, 9, 10, 11, 12, 13, 14, 15, 16, 17, 18, 19, 20 or more days to obtain a cell population comprising at least about 10% cells expressing one or more cortical progenitor marker.

In certain embodiments, the day whereby the cells are contacted with the one or more SMAD inhibitor corresponds to day 0, and the cells are contacted to the inhibitors (i.e., one or more SMAD inhibitor and the one or more Wnt inhibitor) for between days 0 and 5, between days 0 and 6, or between day 0 and day 7.

In certain embodiments, the cells are contacted with the one or more TGFβ/Activin-Nodal inhibitor at a concentration of between about 1 and 20 μM, between about 2 and 18 μM, between about 4 and 16 μM, between about 6 and 14 μM, between about 8 and 12 μM, or about 10 μM. In certain embodiments, the cells are contacted with the one or more TGFβ/Activin-Nodal inhibitor at a concentration of between about 1 and 18 μM, between about 1 and 16 μM, between about 1 and 14 μM, between about 1 and 12 μM, between about 1 and 10 μM, between about 1 and 8 μM, between about 1 and 6 μM. between about 1 and 4 μM, or between about 1 and 2 μM. In certain embodiments, the cells are contacted with the one or more TGFβ/Activin-Nodal inhibitor at a concentration of between about 2 and 20 μM, between about 4 and 20 μM, between about 6 and 20 μM, between about 8 and 20 μM, between about 10 and 20 μM, between about 12 and 20 μM, between about 14 and 20 μM, between about 16 and 20 μM, or between about 18 and 20 μM.

In certain embodiments, the cells are contacted with the one or more BMP inhibitor at a concentration of between about 10 and 500 nM, between about 25 and 475 nM, between about 50 and 450 nM, between about 100 and 400 nM, between about 150 and 350 nM, between about 200 and 300 nM, or about 250 nM or about 100 nM, or about 50 nM. In certain embodiments, the cells are contacted with the one or more BMP inhibitor at a concentration of between about 10 and 475 nM, between about 10 and 450 nM, between about 10 and 400 nM, between about 10 and 350 nM, between about 10 and 300 nM, between about 10 and 250 nM, between about 10 and 200 nM, between about 10 and 150 nM, between about 10 and 100 nM, or between about 10 and 50 nM. In certain embodiments, the cells are contacted with the one or more BMP inhibitor at a concentration of between about 25 and 500 nM, between about 50 and 500 nM, between about 100 and 500 nM, between about 150 and 500 nM, between about 200 and 500 nM, between about 250 and 500 nM, between about 300 and 500 nM, between about 350 and 500 nM, between about 400 and 500 nM, or between about 450 and 500 nM.

In certain embodiments, the cells are contacted with the one or more Wnt inhibitor at a concentration of between about 0.1 and 10 μM, between about 0.5 and 8 μM, between about 1 and 6 μM, between about 2 and 5.5 μM, or about 5 μM, or about 2 μM. or about 1 μM. In certain embodiments, the cells are contacted with the one or more Wnt inhibitor at a concentration of between about 0.1 and 8 μM, between about 0.1 and 6 μM, between about 0.1 and 4 μM, between about 0.1 and 2 μM, between about 0.1 and 1 μM, or between about 0.1 and 0.5 μM. In certain embodiments, the cells are contacted with the one or more Wnt inhibitor at a concentration of between about 0.5 and 10 μM, between about 1 and 10 μM, between about 2 and 0 μM, between about 4 and 10 μM, between about 6 and 10 μM, or between about 8 and 10 μM.

In certain embodiments, the cells are exposed to the one or more Wnt inhibitor concurrently with the one or more SMAD inhibitor. In certain embodiments, the cells are exposed to the one or more Wnt inhibitor for at least about 1, 2, 3, 4, 5, 6, 7, 8, 9, or 10 days or more.

The cortical progenitors can be further differentiated into cortical glial competent cells, e.g., by the methods disclosed in Section 5.2.1.2. The cortical glial competent cells can be further differentiated to cortical astrocytes, e.g., by the methods disclosed in Section 5.2.1.3.

### 5.3.2. In Vitro Differentiation of Stem Cells to Spinal Cord Astrocytes

In certain embodiments, the methods of in vitro differentiation of stem cells to spinal cord progenitors comprise contacting stem cells (e.g., human stem cells) with one or more SMAD inhibitor (e.g., one or more TGFβ/Activin-Nodal inhibitor, and/or one or more BMP inhibitor) one or more activator of retinoic acid (“RA”) signaling (referred to as “RA activator”), and one or more activator of Sonic hedgehog signaling (referred to as “SHH activator”) to obtain a cell population comprising at least about 10% cells expressing one or more spinal cord progenitor marker. Non-limiting examples of RA activators include RA, retinol, retinal, tretinoin, isotretinoin, alitretinoin, etretinate, acitretin, tazarotene, bexarotene, adapalene, those disclosed in International Publication No. WO/2017/112901 filed Dec. 22, 2016).

As used herein, the teen “Sonic hedgehog (SHH or Shh)” refers to a protein that is one of at least three proteins in the mammalian signaling pathway family called hedgehog, another is desert hedgehog (DHH) wile a third is Indian hedgehog (IHH). SHH interacts with at least two transmembrane proteins by interacting with transmembrane molecules Patched (PTC) and Smoothened (SMO). SHH typically binds to PTC which then allows the activation of SMO as a signal transducer. In the absence of SHH, PTC typically inhibits SMO, which in turn activates a transcriptional repressor so transcription of certain genes does not occur. When SHH is present and binds to PTC, PTC cannot interfere with the functioning of SMO. With SMO uninhibited, certain proteins are able to enter the nucleus and act as transcription factors allowing certain genes to be activated (see, Gilbert, 2000 Developmental Biology (Sunderland, Mass.: Sinauer Associates, Inc., Publishers).

Non-limiting examples of SHH activators include, molecules that bind to PTC, molecules that bind to SMO. Non-limiting examples of molecules that bind to PTC include SHH, recombinant SHH (e.g., N-terminal SHH, e.g., SHH C25II, SHH C24II). Non-limiting examples of molecules that bind to SMO include SMO agonists (e.g., purmorphamine). As used herein, the term “Sonic hedgehog (SHH) C25II” refers to a recombinant N-Terminal fragment of a full-length murine sonic hedgehog protein capable of binding to the SHH receptor for activating SHH, one example is R and D Systems catalog number: 464-5H-025/CF.

As used herein, the term “purmorphamine” refers to a purine derivative, such as CAS Number: 483367-10-8, for one example see structure below, that activates the Hedgehog pathway including by targeting Smoothened.

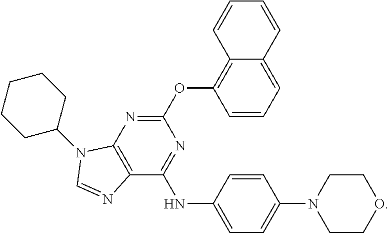 [SMILES: c1ccc2c(Oc3nc(Nc4ccc(N5CCOCC5)cc4)c4ncn(C5CCCCC5)c4n3)cccc2c1]

In certain embodiments, purmorphamine is Stemolecule™ Purmorphamine, Stemgent, Inc. Cambridge, Mass., United States, Any SHH activators disclosed in International Publication No. WO/2013/067362, filed Nov. 2, 2012 can be used in the methods described herein.

Non-limiting examples of spinal cord progenitor markers FOXG1, SOX2, NESTIN, and TBR2.

In certain embodiments, the cells are contacted with the one or more SMAD inhibitor (e.g., one or more TGFβ/Activin-Nodal inhibitor, and/or one or more BMP inhibitor), the one or more RA activator, and the one or more SHH activator for at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20 or more days, and/or up to about 1, up to about 2, up to about 3, up to about 4, up to about 5, up to about 6, up to about 7, up to about 8, up to about 9, up to about 10, up to about 11, up to about 12, up to about 13, up to about 14, up to about 15, up to about 16, up to about 17, up to about 18, up to about 19, up to about 20 or more days to obtain a cell population comprising at least about 10% cells expressing one or more spinal cord progenitor marker. In certain embodiments, the cells are contacted with the one or more SMAD inhibitor, the one or more RA activator, and the one or more SHH activator for about 1, 2, 3, 4, 5, 6, 7, 8, 9, 10, 11, 12, 13, 14, 15, 16, 17, 18, 19, 20 or more days.

In certain embodiments, the day whereby the cells are contacted with the one or more SMAD inhibitor corresponds to day 0, and the cells are contacted to the one or more SMAD inhibitor, the one or more RA activator, and the one or more SHH activator between days 1 and 12.

In certain embodiments, the cells are exposed to the one or more RA activator and the one or more SHH activator concurrently with the one or more SMAD inhibitor. In certain embodiments, the initial exposure of the cells to the one or more RA activator and the one or more SHH activator is about 1, 2, 3, 4, 5 or more days from the initial exposure of the cells to the one or more SMAD inhibitor.

In certain embodiments, the cells are exposed to the one or more RA activator and the one or more SHH activator for at least about 1, 2, 3, 4, 5, 6, 7, 8, 9, 10, 11, 12, 13, 14, 15 days or more.

In certain embodiments, the cells are contacted with the one or more TGFβ/Activin-Nodal inhibitor at a concentration of between about 1 and 20 μM, between about 2 and 18 μM, between about 4 and 16 μM, between about 6 and 14 μM, between about 8 and 12 μM, or about 10 μM. In certain embodiments, the cells are contacted with the one or more TGFβ/Activin-Nodal inhibitor at a concentration of between about 1 and 18 μM, between about 1 and 16 μM, between about 1 and 14 μM, between about 1 and 12 μM, between about 1 and 10 μM, between about 1 and 8 μM, between about 1 and 6 μM. between about 1 and 4 μM, or between about 1 and 2 μM. In certain embodiments, the cells are contacted with the one or more TGFβ/Activin-Nodal inhibitor at a concentration of between about 2 and 20 μM, between about 4 and 20 μM, between about 6 and 20 μM, between about 8 and 20 μM, between about 10 and 20 μM, between about 12 and 20 μM, between about 14 and 20 μM, between about 16 and 20 μM, or between about 18 and 20 μM.

In certain embodiments, the cells are contacted with the one or more BMP inhibitor at a concentration of between about 10 and 500 nM, between about 25 and 475 nM, between about 50 and 450 nM, between about 100 and 400 nM, between about 150 and 350 nM, between about 200 and 300 nM, or about 250 nM or about 100 nM, or about 50 nM. In certain embodiments, the cells are contacted with the one or more BMP inhibitor at a concentration of between about 10 and 475 nM, between about 10 and 450 nM, between about 10 and 400 nM, between about 10 and 350 nM, between about 10 and 300 nM, between about 10 and 250 nM, between about 10 and 200 nM, between about 10 and 150 nM, between about 10 and 100 nM, or between about 10 and 50 nM. In certain embodiments, the cells are contacted with the one or more BMP inhibitor at a concentration of between about 25 and 500 nM, between about 50 and 500 nM, between about 100 and 500 nM, between about 150 and 500 nM, between about 200 and 500 nM, between about 250 and 500 nM, between about 300 and 500 nM, between about 350 and 500 nM, between about 400 and 500 nM, or between about 450 and 500 nM.

In certain embodiments, the cells are contacted with the one or more RA activator (e.g., RA) at a concentration of between about 0.1 and 10 μg/ml, between about 0.1 and 5 μg/ml, between about 0.1 and 4 μg/ml, between about 0.1 and 3 μg/ml, between about 0.1 and 2 μg/ml, between about 0.1 and 1 μg/ml. In certain embodiments, the cells are contacted with the one or more RA activator (e.g., RA) at a concentration of about 1 μg/ml to produce spinal cord progenitors.

The spina cord progenitors can be further differentiated into spinal cord glial competent cells, e.g., by the methods disclosed in Section 5.2.1.2. The spinal cord glial competent cells can be further differentiated to spinal cord astrocytes, e.g., by the methods disclosed in Section 5.2.1.3.

The differentiated astrocytes, or precursors thereof, can be purified after differentiation, e.g., in a cell culture medium. As used herein, the terms “purified,” “purify,” “purification,” “isolated,” “isolate,” and “isolation” refer to the reduction in the amount of at least one contaminant from a sample. For example, a desired cell type is purified by at least 10%, by at least 30%, by at least 50%, by at least 75%, by at least 90%, by at least 95%, by at least 99%, by at least 99.5%, or by at least 99.9% or more, with a corresponding reduction in the amount of undesirable cell types. The term “purify” can refer to the removal of certain cells (e.g., undesirable cells) from a sample. The removal or selection of non-astrocyte cells, or precursors thereof, results in an increase in the percent of desired cells in the sample. In certain embodiments, the cells are purified by sorting a mixed cell population into cells expressing at least one astrocyte marker. In certain embodiments, the cells are purified by sorting a mixed cell population into cells expressing at least one astrocyte marker, e.g., GFAP, AQP4, CD44, S100b, SOX9, GLT-1, CSRP1 and/or NFIA, or combinations thereof.

### 5.4. Compositions Comprising Differentiated Cell Populations

The presently disclosed subject matter further provides compositions comprising a population of differentiated NSCs produced by the in vitro differentiation methods described herewith. Furthermore, the presently disclosed subject matter provides compositions comprising a population of glial competent cells differentiated from the in vitro differentiated NSCs by the methods described herewith. Additionally, the presently disclosed subject matter provides compositions comprising a population of astrocytes differentiated or matured from the in vitro differentiated glial competent cells by the methods described herewith. In certain embodiments, the glial competent cells are astrocyte precursor cells.

In certain embodiments, the presently disclosed subject matter provides compositions comprising a population of in vitro differentiated cells, wherein at least about 50% (e.g., at least about 55%, at least about 60%, at least about 70%, at least about 75%, at least about 80%, at least about 85%, at least about 90%, at least about 95%, or at least about 99%) of the population of cells express one or more NSC marker and wherein less than about 25% (e.g., less than about 20%, less than about 15%, less than about 10%, less than about 5%, less than about 4%, less than about 3%, less than about 2%, less than about 1%, less than about 0.5%, or less than about 0.1%) of the population of cells express one or more stem cell marker.

Furthermore, the presently disclosed subject matter provides compositions comprising a population of in vitro differentiated cells, wherein at least about 50% (e.g., at least about 55%, at least about 60%, at least about 70%, at least about 75%, at least about 80%, at least about 85%, at least about 90%, at least about 95%, or at least about 99%) of the population of cells express one or more glial competent NSC marker or glial competent cell marker, and wherein less than about 25% (e.g., less than about 20%, less than about 15%, less than about 10%, less than about 5%, less than about 4%, less than about 3%, less than about 2%, less than about 1%, less than about 0.5%, or less than about 0.1%) of the population of cells express one or more marker selected from the group consisting of stem cell markers, NSC markers, and neuronal markers.

Furthermore, the presently disclosed subject matter provides compositions comprising a population of in vitro differentiated cells, wherein at least about 50% (e.g., at least about 55%, at least about 60%, at least about 70%, at least about 75%, at least about 80%, at least about 85%, at least about 90%, at least about 95%, or at least about 99%) of the population of cells express one or more astrocyte marker, and wherein less than about 25% (e.g., less than about 20%, less than about 15%, less than about 10%, less than about 5%, less than about 4%, less than about 3%, less than about 2%, less than about 1%, less than about 0.5%, or less than about 0.1%) of the population of cells express one or more marker selected from the group consisting of stem cell markers, NSC markers, neuronal markers, and glial competent cell markers.

Non-limiting examples of stem cell markers include OCT4, NANOG, SOX2, LIN28, SSEA4 and SSEA3.

Non-limiting examples of neural stem cell markers include PAX6, NESTIN, SOX1, SOX2, PLZF, ZO-1, and BRN2. In certain embodiments, the neural stem cell marker is selected from the group consisting of PAX6, SOX1, PLZF, and ZO-1.

Non-limiting examples of glial competent cell markers include CD44, AQP4, SOX2 and NESTIN. In certain embodiments, the glial competent cell marker is selected from the group consisting of CD44 and AQP4.

Non-limiting examples of neuronal markers include Tuj1, MAP2, and DCX.

Non-limiting examples of astrocyte markers include GFAP, AQP4, CD44, S100b, SOX9, NFIA, GLT-1 and CSRP1.

In certain embodiments, the differentiated cell population is derived from a population of human stem cells. The presently disclosed subject matter further provides for compositions comprising such differentiated cell population.

In certain embodiments, the composition comprises a population of from about 1×10⁴ to about 1×10¹⁰, from about 1×10⁴ to about 1×10⁵, from about 1×10⁵ to about 1×10⁹, from about 1×10⁵ to about 1×10⁶, from about 1×10⁵ to about 1×10⁷, from about 1×10⁶ to about 1×10⁷, from about 1×10⁶ to about 1×10⁸, from about 1×10⁷ to about 1×10⁸, from about 1×10⁸ to about 1×10⁹, from about 1×10⁸ to about 1×10¹⁰, or from about 1×10⁹ to about 1×10¹⁰ of the presently disclosed stem-cell-derived glial competent cells or astrocytes are administered to a subject. In certain embodiments, the composition comprises a population from about 1×10⁵ to about 1×10⁷ the presently disclosed glial competent cells or astrocytes. In certain embodiments, the glial competent cells are astrocyte precursor cells In certain embodiments, said composition is frozen. In certain embodiments, said composition may further comprise one or more cryoprotectant, for example, but not limited to, dimethylsulfoxide (DMSO), glycerol, polyethylene glycol, sucrose, trehalose, dextrose, or a combination thereof.

In certain non-limiting embodiments, the composition further comprises a biocompatible scaffold or matrix, for example, a biocompatible three-dimensional scaffold that facilitates tissue regeneration when the cells are implanted or grafted to a subject. In certain non-limiting embodiments, the biocompatible scaffold comprises extracellular matrix material, synthetic polymers, cytokines, collagen, polypeptides or proteins, polysaccharides including fibronectin, laminin, keratin, fibrin, fibrinogen, hyaluronic acid, heparin sulfate, chondroitin sulfate, agarose or gelatin, and/or hydrogel. (See, e.g., U.S. Publication Nos. 2015/0159135, 2011/0296542, 2009/0123433, and 2008/0268019, the contents of each of which are incorporated by reference in their entireties).

In certain embodiments, the composition is a pharmaceutical composition that comprises a pharmaceutically acceptable carrier, excipient, diluent or a combination thereof. The compositions can be used for preventing and/or treating neurodegenerative disorders, as described herein.

The presently disclosed subject matter also provides a device comprising the differentiated cells or the composition comprising thereof, as disclosed herein. Non-limiting examples of devices include syringes, fine glass tubes, stereotactic needles and cannulas.

### 5.5 Methods of Preventing and/or Treating Neurodegenerative Disorders

The in vitro differentiated cells that express one or more astrocyte marker (also referred to as “stem-cell-derived astrocytes”), or precursors thereof, can be used for preventing and/or treating a neurodegenerative disorder. The presently disclosed subject matter provides for methods of preventing and/or treating a neurodegenerative disorder comprising administering an effective amount of the presently disclosed stem-cell-derived astrocytes, and/or precursors thereof, or a composition comprising thereof into a subject suffering from a neurodegenerative disorder. Non-limiting examples of neurodegenerative disorder include Parkinson's disease, Alzheimer's disease, amyotrophic lateral sclerosis (ALS), and Rett syndrome. The presently disclosed subject matter also provides for methods of reducing severity of damage due to neurological injury, for example, ischemia or stroke, comprising administering an effective amount of the presently disclosed stem-cell-derived astrocytes, and/or precursors thereof, or a composition comprising thereof.

The presently disclosed stem-cell-derived astrocytes, and precursors thereof, or a composition comprising thereof can be administered or provided systemically or directly to a subject for treating or preventing a neurodegenerative disorder or reducing damage due to neurological injury. In certain embodiments, the presently disclosed stem-cell-derived astrocytes, and precursors thereof, or a composition comprising thereof are directly injected into an organ of interest (e.g., the central nervous system (CNS)).

The presently disclosed stem-cell-derived astrocytes, and precursors thereof, or a composition comprising thereof can be administered in any physiologically acceptable vehicle. Pharmaceutical compositions comprising the presently disclosed stem-cell-derived cells and a pharmaceutically acceptable carrier are also provided. The presently disclosed stem-cell-derived astrocytes, and precursors thereof, and the pharmaceutical compositions comprising thereof can be administered via localized injection, orthotopic (OT) injection, systemic injection, intravenous injection, or parenteral administration. In certain embodiments, the presently disclosed stem-cell-derived astrocytes, and precursors thereof, are administered to a subject suffering from a neurodegenerative disorder via orthotopic (OT) injection.

The presently disclosed stem-cell-derived astrocytes, and precursors thereof, and the pharmaceutical compositions comprising thereof can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof. Sterile injectable solutions can be prepared by incorporating the compositions of the presently disclosed subject matter, e.g., a composition comprising the presently disclosed stem-cell-derived precursors, in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, alum inurn monostearate and gelatin. According to the presently disclosed subject matter, however, any vehicle, diluent, or additive used would have to be compatible with the presently disclosed stem-cell-derived astrocytes, and precursors thereof.

Viscosity of the compositions, if desired, can be maintained at the selected level using a pharmaceutically acceptable thickening agent. Methylcellulose can be used because it is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

Those skilled in the art will recognize that the components of the compositions should be selected to be chemically inert and will not affect the viability or efficacy of the presently disclosed stem-cell-derived astrocytes, and precursors thereof. This will present no problem to those skilled in chemical and pharmaceutical principles, or problems can be readily avoided by reference to standard texts or by simple experiments (not involving undue experimentation), from this disclosure and the documents cited herein.

One consideration concerning the therapeutic use of the presently disclosed stem-cell-derived astrocytes, and precursors thereof, is the quantity of cells necessary to achieve an optimal effect. An optimal effect includes, but are not limited to, repopulation of central nervous system (CNS) or peripheral nervous system (PNS) regions of a subject suffering from a neurodegenerative disorder, and/or improved function of the subject's CNS and/or PNS.

An “effective amount” (or “therapeutically effective amount”) is an amount sufficient to affect a beneficial or desired clinical result upon treatment. An effective amount can be administered to a subject in one or more doses. In terms of treatment, an effective amount is an amount that is sufficient to palliate, ameliorate, stabilize, reverse or slow the progression of the neurodegenerative disorder, or otherwise reduce the pathological consequences of the neurodegenerative disorder, or reduce severity of damage to due neurological injury. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the cells administered.

In certain embodiments, an effective amount of the presently disclosed stem-cell-derived astrocytes, and precursors thereof, is an amount that is sufficient to repopulate CNS or PNS regions of a subject suffering from a neurodegenerative disorder.

In certain embodiments, an effective amount of the presently disclosed stem-cell-derived astrocytes, and precursors thereof, is an amount that is sufficient to improve the function of the CNS and/or PNS of a subject suffering from a neurodegenerative disorder, or who has experienced a neurological injury, e.g., the improved function can be about 1%, about 5%, about 10%, about 20%, about 30%, about 40%, about 50%, about 60%, about 70%, about 80%, about 90%, about 95%, about 98%, about 99% or about 100% of the function of a normal person's CNS and/or PNS.

The quantity of cells to be administered will vary for the subject being treated. In certain embodiments, from about 1×10⁴ to about 1×10¹⁰, from about 1×10⁴ to about 1×10⁵, from about 1×10⁵ to about 1×10⁹, from about 1×10⁵ to about 1×10⁶, from about 1×10⁵ to about 1×10⁷, from about 1×10⁶ to about 1×10⁷, from about 1×10⁶ to about 1×10⁸, from about 1×10⁷ to about 1×10⁸, from about 1×10⁸ to about 1×10⁹, from about 1×10⁸ to about 1×10¹⁰, or from about 1×10⁹ to about 1×10¹⁰ the presently disclosed stem-cell-derived cells.

### 5.6 Kits

The presently disclosed subject matter provides for kits for inducing differentiation of stem cells. In certain embodiments, the kit comprises one or more of the following: (a) one or more inhibitor of transforming growth factor beta (TGFβ)/Activin-Nodal signaling, (b) one or more inhibitor of BMP signaling, (c) one or more NFIA activator, (d) LIF, one or more derivative, analog, and/or activator thereof, (e) FBS, and (f) instructions for inducing differentiation of the stem cells into a population of differentiated cells that express one or more astrocyte marker, or precursor cells thereof.

The presently discloses subject matter also provides for kits comprising a population of differentiated cells that express one or more astrocyte marker, or precursor cells thereof, wherein the cells are prepared according to the methods described herein. In certain embodiments, the cells are comprised in a pharmaceutical composition.

### 6. EXAMPLES

The presently disclosed subject matter will be better understood by reference to the following Example, which is provided as exemplary of the presently disclosed subject matter, and not by way of limitation.

### 6.1 Example 1: Methods of Preparing Stem Cell-Derived Astrocytes by Modulating NFIA Expression Levels in a Population of Stem Cells

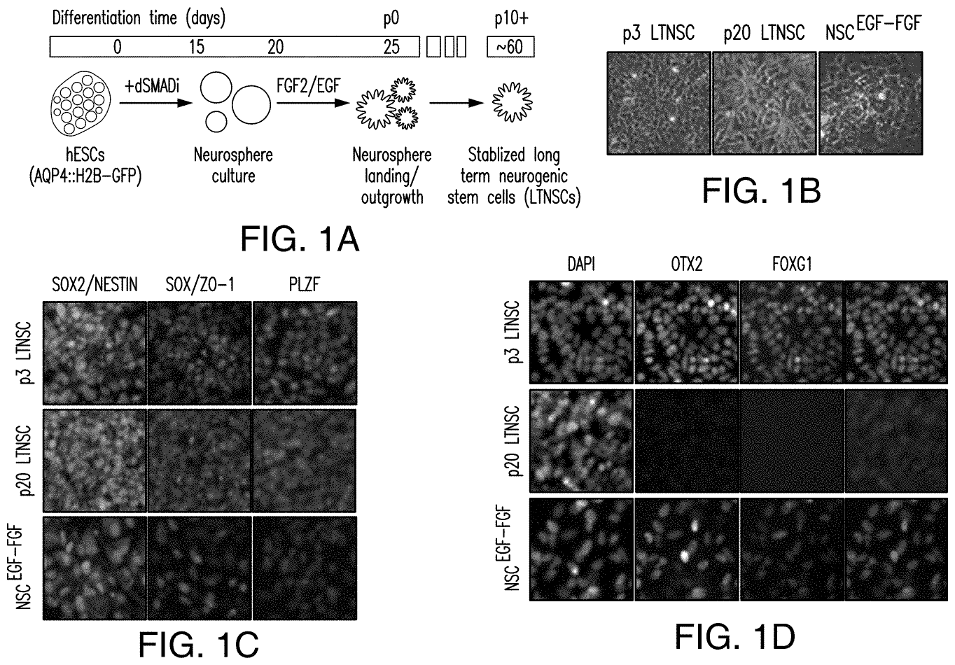

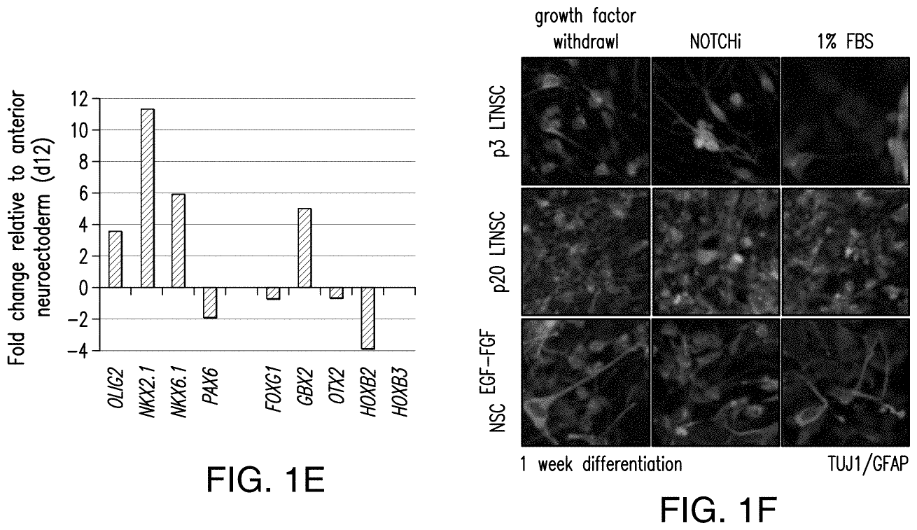

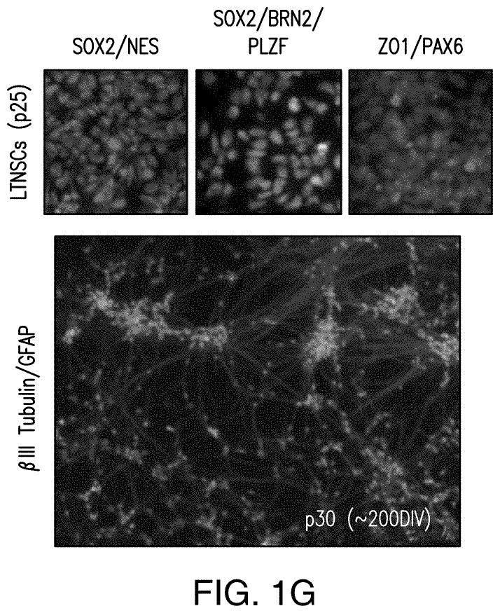

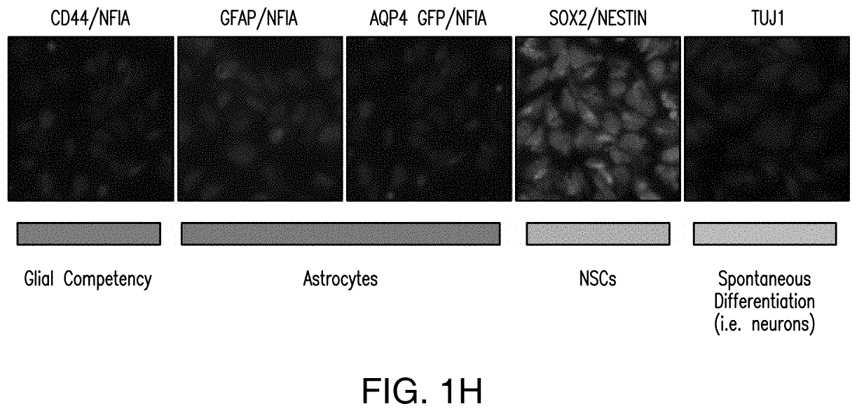

LTNSCs (or LT-hESCNSCs) were derived from human pluripotent stem cells using the dual SMAD inhibition method (SB431542, an inhibitor of TGFβ/Activin-Nodal signaling, and LDN193189, an inhibitor of BMP signaling) (Chambers et al., Nature Biotechnology 27, 275-280 (2009)). Cells were cultured for 12 days as a monolayer and then placed into a neurosphere culture (non-adherent plates) containing 20 ng/ml of FGF2 and 20 ng/ml EGF. Spheres were then landed onto Poly-ornathine, Laminin and Fibronectin coated dishes to allow for outgrowth. Cells were continually passaged for roughly 10 passages before determined to be LTNSCs (FIG. 1A). The morphology of the LTNSCs resembled a very early neuroectoderm (FIG. 1B). LTNSCs expressed several key neural stem cell (NSC) markers such as SOX2, NESTIN and SOX1 but also expressed PLZF and displayed a focal expression of ZO-1 indicating their rosette or early NSC nature (FIG. 1C). LTNSCs lose their regional identity over time as they begin expressing forebrain markers, but become more caudal and ventralized with longer culture times, represented by GBX2 and NKX2.1 expression, respectively (FIGS. 1D and 1E). This population of cells rapidly became neurons despite the differentiation inducing media, even under FBS conditions (FIG. 1F).

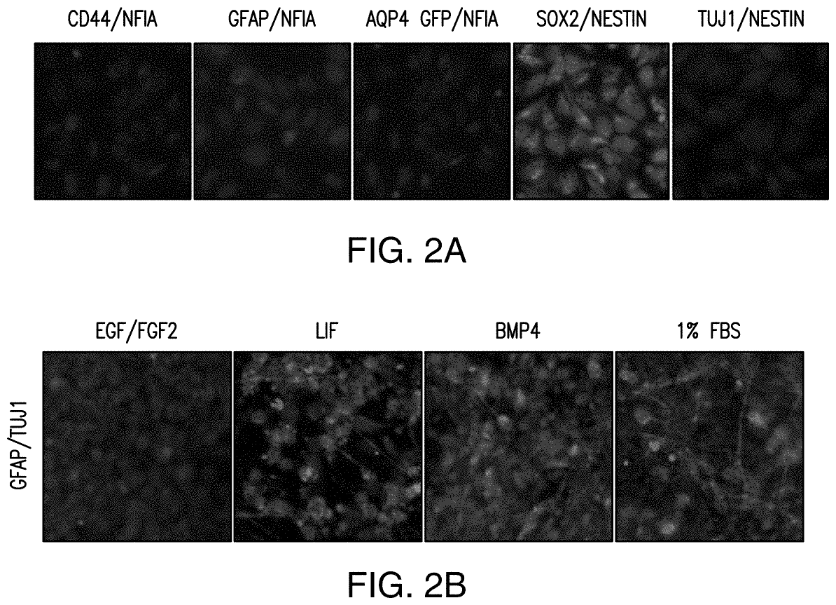

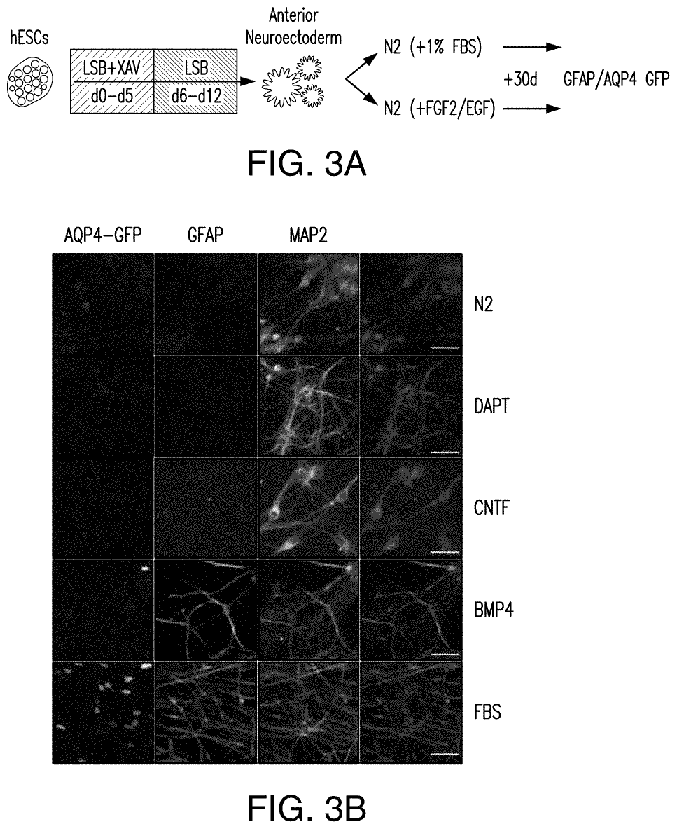

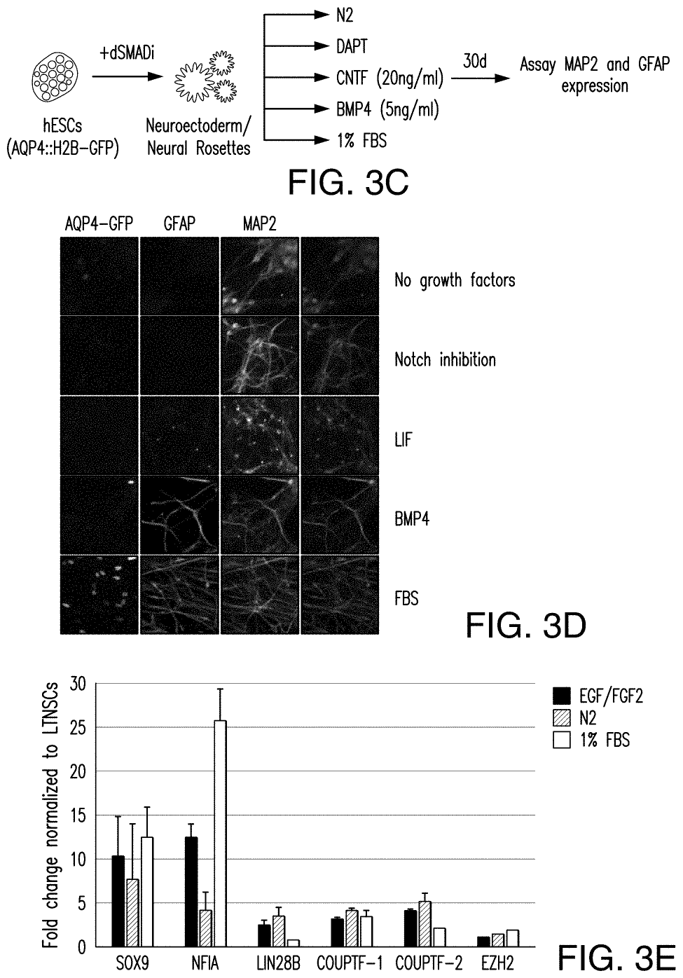

The LTNSC population did not express markers that suggest glial competency (i.e., markers of glial competent cells) such as CD44, GFAP, and AQP4. These cells were also TUJ1 negative implying their homogenous nature of being nearly 100% neural stem cells (FIG. 2A). When LTNSCs were cultured in media containing gliogenic molecules (i.e. LIF and BMP4), these cells became neurons rather than GFAP+ astrocytes (FIG. 2B) in the short term. However, when either neural rosettes or LTNSCs were cultured in various conditions including LIF/CNTF, BMP4 or FBS for longer culture times (roughly 30 days), some cells acquired glial competency in the FBS condition, identified by the expression of AQP4 and GFAP. In the FBS condition, the expression of gliogenic factors was assessed and expression of SOX9 and NFIA was more enriched when cultured with FBS compared to the other treatments (FIG. 3).

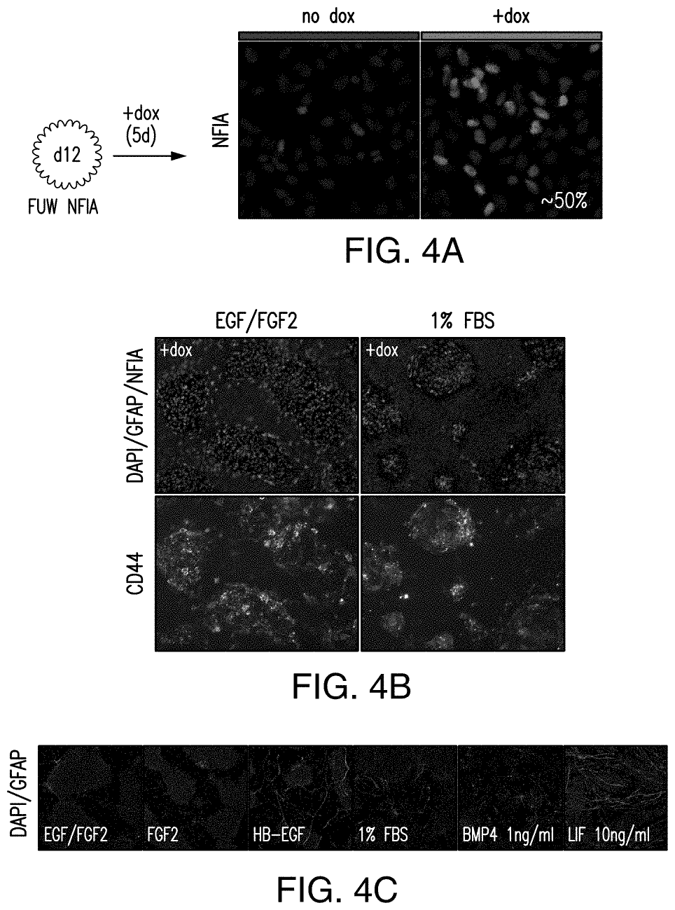

The effect of NFIA expression on the differentiation of stem cells into astrocytes was examined. Overexpression of NFIA was achieved by cloning the NFIA cDNA into a doxycycline inducible vector. Cells were subjected to viral infection with the construct and assessed for the expression of NFIA (FIG. 4A). After 7 days of doxycycline treatment, there was a large morphological change in the cell population where the NFIA induced cells expressed CD44 (a cell surface marker). The expression of GFAP was observed when the cells were cultured in FBS (FIG. 4B). After removing the expression of NFIA, it was determined whether glial competent factors were now able to promote the differentiation of the NFIA exposed cells towards astrocytes. LIF, BMP4, and FBS enhanced the expression of GFAP especially in the absence of FGF2 (FIG. 4C).

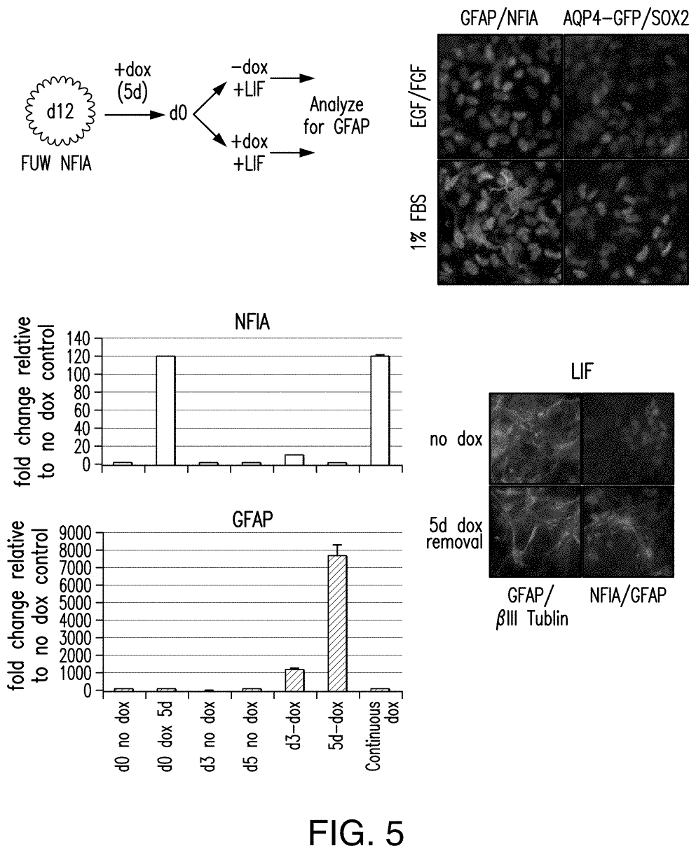

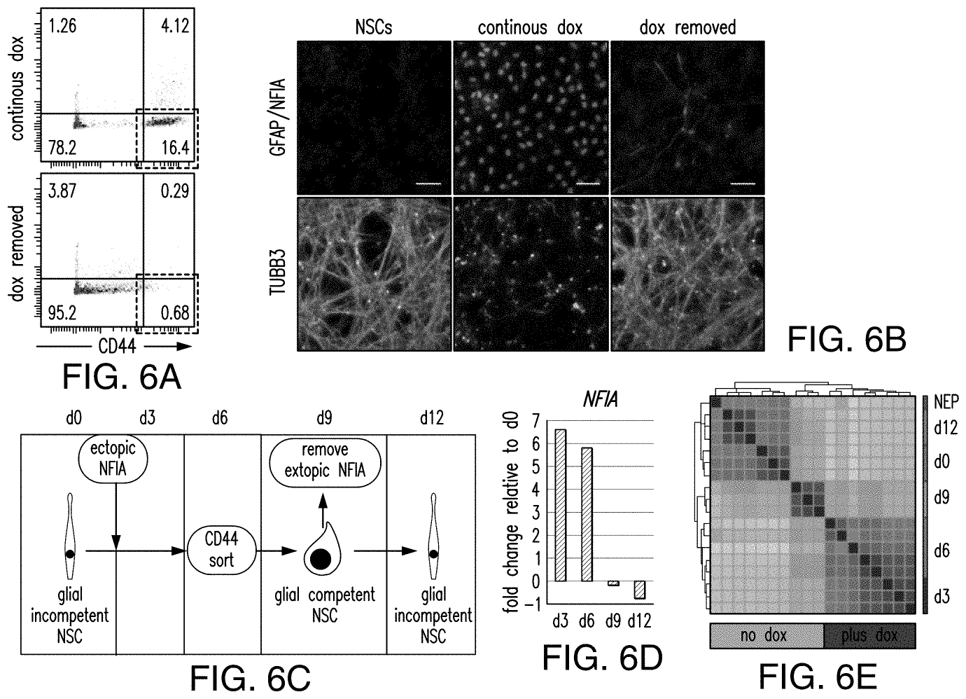

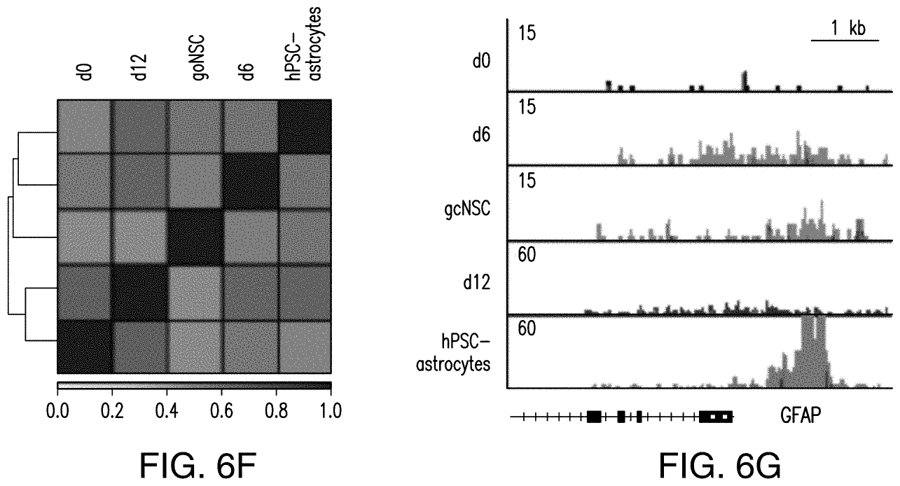

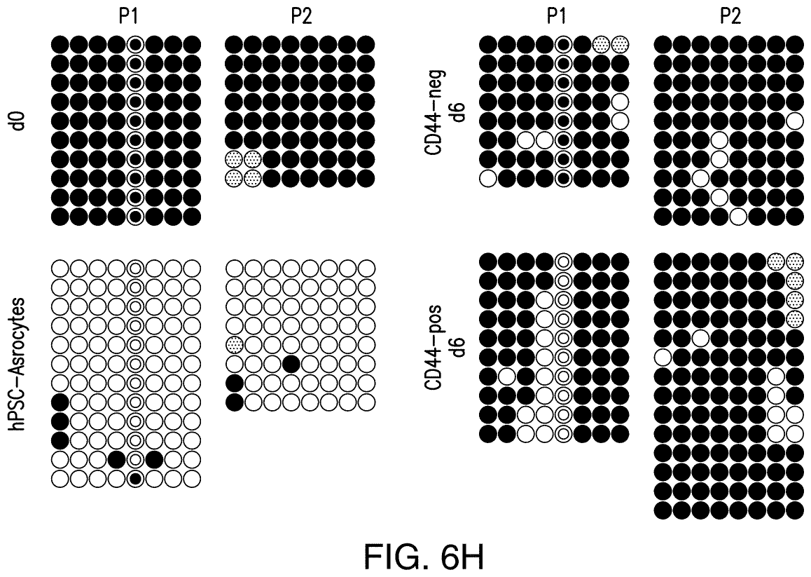

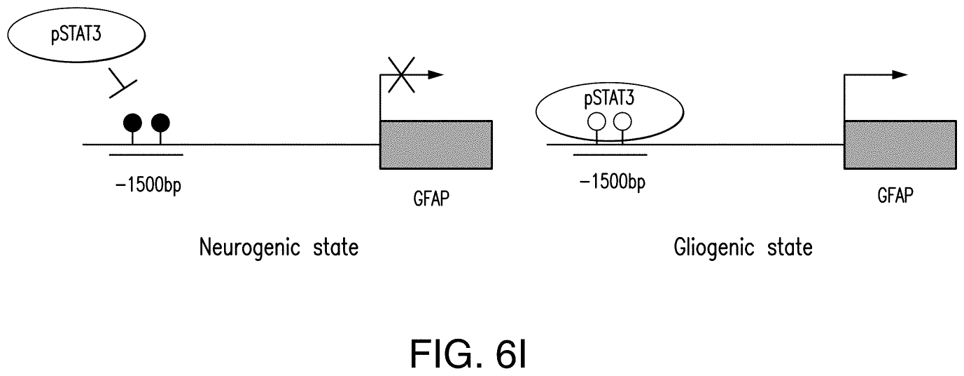

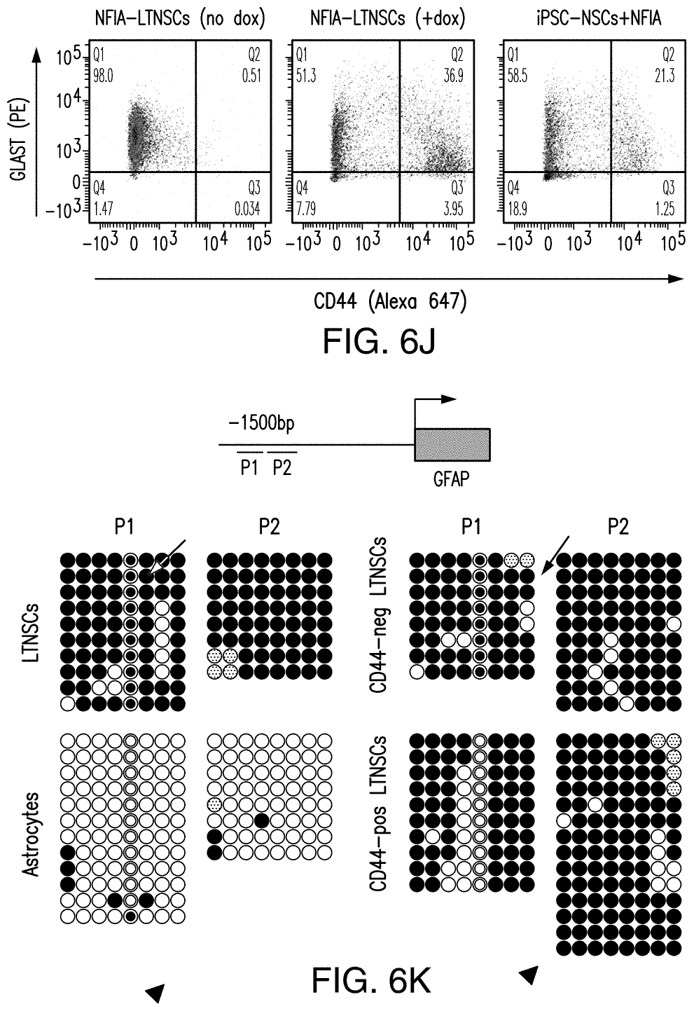

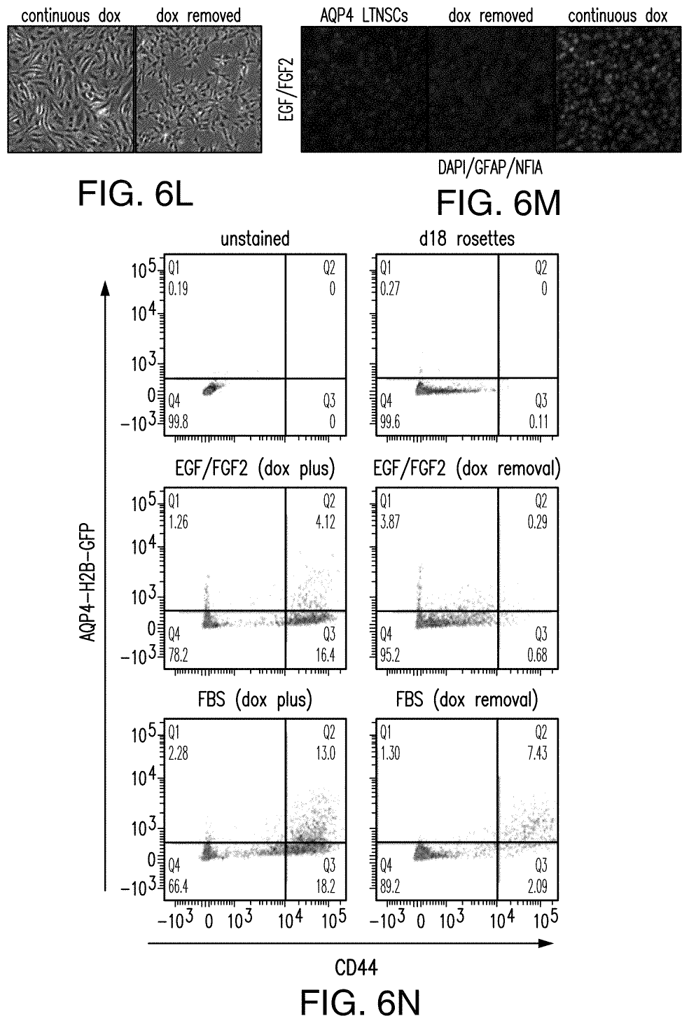

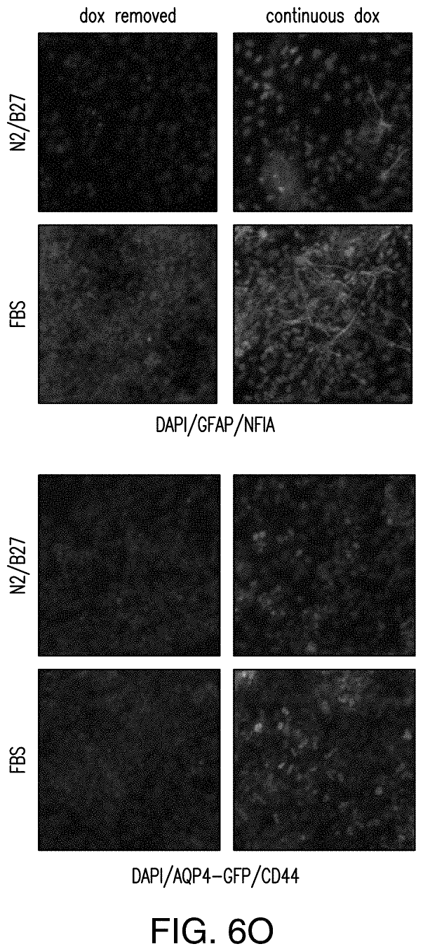

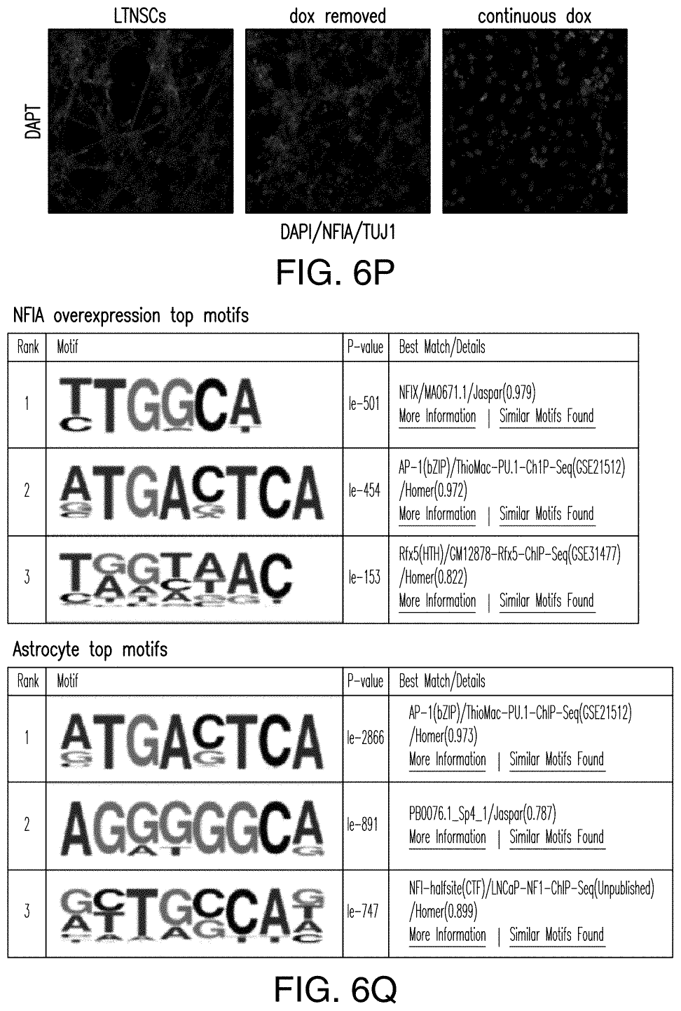

NFIA allows for acquisition of glial competency, but is inhibitory to glial differentiation. Cells induced with NFIA cannot upregulate GFAP (FIG. 5). However, a subsequent decrease of NFIA expression and culture in LIF increased the expression of GFAP. Increasing NFIA expression did induce a corresponding increase in CD44 expression, a marker of glial competent cells. GFAP expression in neurogenic cells is blocked by methylation at the STAT3 CpG site, while this site is demethylated in glial cells, wherein GFAP is expressed. The STAT3 CpG site was demethylated in CD44+ cells induced by NFIA (FIG. 6).

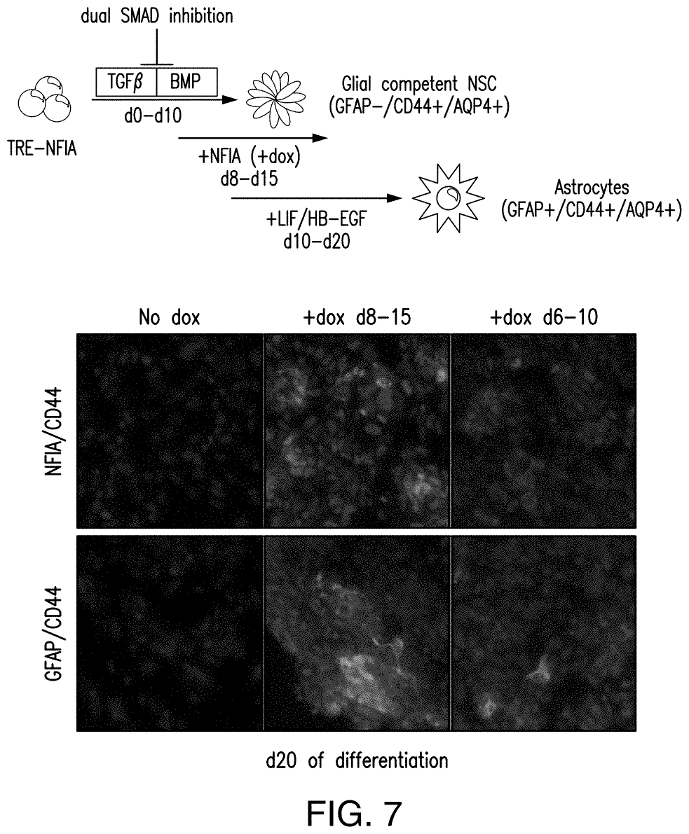

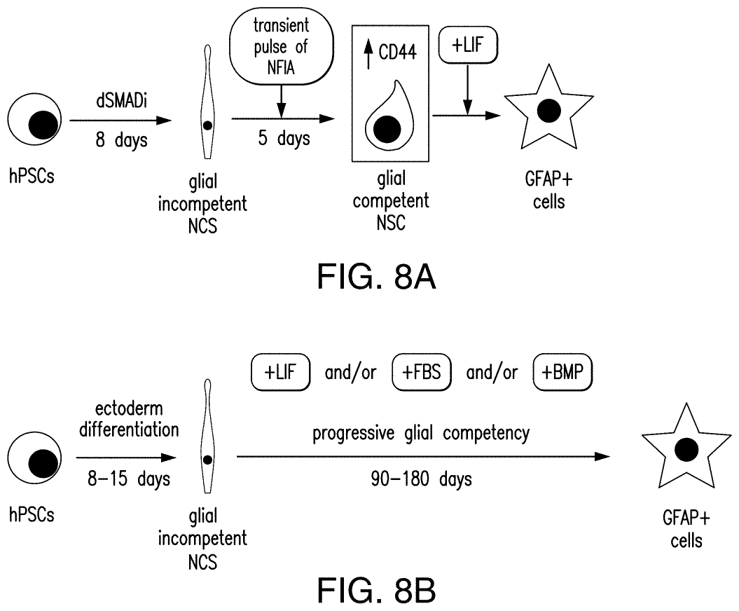

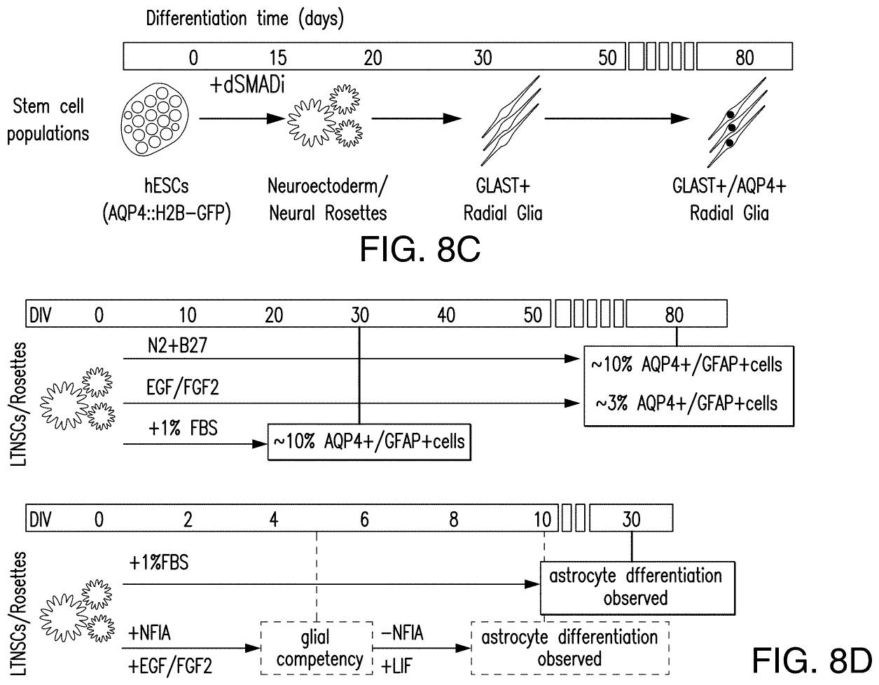

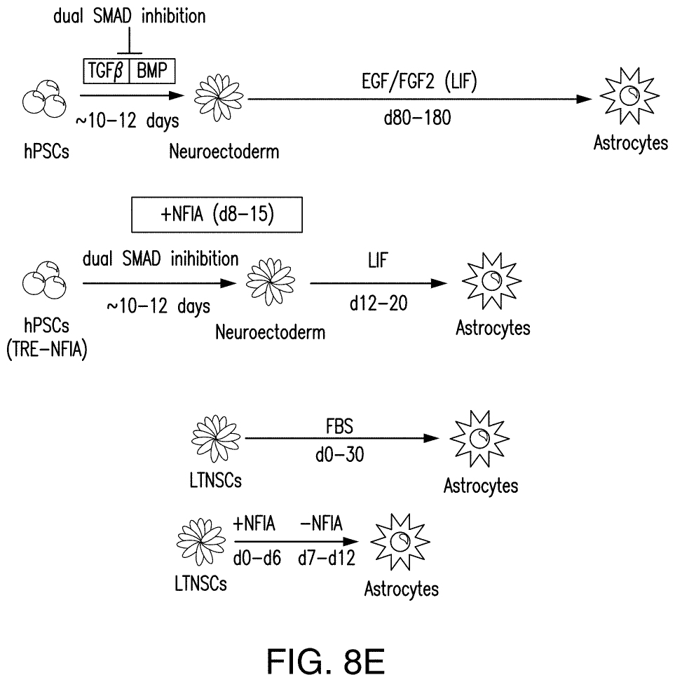

A recombinant NFIA-inducible hESC line under the control of doxycycline was also created. Using dual SMAD inhibition, glial competent NSCs and astrocytes were generated in about 20 days of culture (FIG. 7), thus accelerating the differentiation by 3.5-10 folds compared to other protocols, such as culture in EGF/FGF2. FIG. 8 is a summary of cell culture protocols and timelines for differentiating stem cells into astrocytes.

As described by the present example, increasing the expression level of NFIA in stem cells increased the expression of glial competent cell markers, but differentiation into astrocytes was inhibited. Reducing NFIA expression in the glial competent cells allowed for differentiation of the cells into astrocytes.

### 6.2 Example 2: Wild Type Astrocytes Reduce Cell Death of Spinal Motor Neurons (sMN) and Ocular Motor Neurons (oMN)

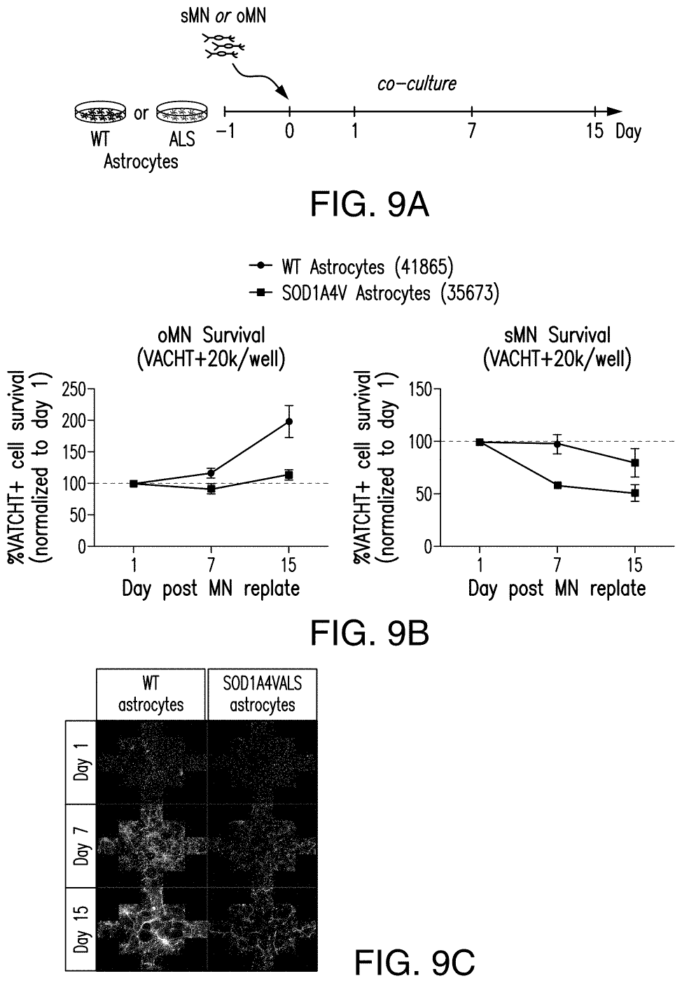

The SOD1 mutation A4V is one cause of amyotrophic lateral sclerosis (ALS) and the resulting cell death of sMN and oMN cells. To examine whether the presence of SOD1A4V mutant astrocytes affect sMN and oMN cell survival, sMN and oMN cells were cultured with astrocytes differentiated as described above from either wild type or wild type SOD1A4V precursors. sMN and oMN were cultured with astrocytes for up to 15 days, and cell survival was determined by measuring VACHT+ cells. Culturing oMN with wild type astrocytes increased survival of the motor neurons compared to culture with SOD1A4V astrocytes, while the wild type astrocytes reduced cell death of sMN compared to SOD1A4V astrocytes (FIG. 9).

### 6.3 Example 3: NFIA Enables Rapid Derivation of Functional Human Astrocytes from Pluripotent Stem Cells by Modulating G1 Cell Cycle Length

Summary

Astrocytes are the most abundant glial cell type in the human brain, and their dysfunction is a driver in the pathogenesis of both neurodevelopmental and neurodegenerative disorders¹. Astrocytes are derived from late neural stem cells (NSCs). During early development, NSCs are fate-restricted to exclusively produce neurons, while at later stages, they undergo a switch from neurogenic to gliogenic competency resulting in progressive production of astrocytes and oligodendrocytes². The molecular nature of the gliogenic switch has remained elusive, and its timing varies dramatically across species from 7 days in the mouse to 6-9 months during human development³. Those species-specific timing differences similarly apply to NSCs derived from human pluripotent stem cells (hPSCs)⁴. The highly protracted timing of acquiring glial competency in hPSCs presents a major roadblock in the quest for deriving human astrocytes for basic and translational applications

Here, it was identified that Nuclear Factor IA (NFIA) as the molecular switch for inducing human glial competency. Transient expression of NFIA for 5 days, in the presence of glial-promoting factors, was sufficient to generate hPSC-derived astrocytes as compared to 3-6 months of differentiation using current protocols. NFIA-induced astrocytes promoted synaptogenesis, exhibited neuroprotective properties, displayed calcium transients in response to appropriate stimuli, and engrafted in the adult brain. Finally, NFIA-induced astrocytes were induced to express features of activated astrocytes. The underlying mechanism of NFIA-induced glial competency involves rapid but reversible chromatin remodeling, GFAP promoter demethylation, and a striking lengthening of the G1 phase in the cell cycle. Genetic or pharmacological manipulation of G1 length partly mimicked NFIA function in glial competency. This study addressed a significant roadblock in hPSC and glial biology by defining key mechanistic features of the gliogenic switch and by enabling the rapid production of human astrocytes for disease modeling and regenerative medicine.

Methods and Materials

### Cell Culture

Human pluripotent stem cells were maintained on irradiated mouse embryonic fibroblasts (Global Stem) in the stem cell maintenance media as previously described (Chambers et al.) containing 10 ng/ml FGF2 (R&D Systems, 233-FB-001MG/CF). Cells were subjected to mycoplasma testing every 2-3 months. Neural stem cells and glial progenitors were maintained on Poly-ornithine (PO), Laminin (Lam) and Fibronectin (FN) coated dishes in NSC media consisting of N2 media with 10 ng/ml FGF2, 10 ng/ml EGF and 1:1000 B27 supplement. Astrocytes were maintained on PO/Lam/FN coated dishes in astrocyte media consisting of N2 media with 10 ng of HB-EGF (R&D Systems, 259-HE).

Derivation of Anterior Forebrain Neuroectoderm from hPSCs

Human pluripotent stem cells (2.5-3.0×10⁵ cells/cm²) were dissociated into single cells and plated onto Matrigel (BD Biosciences) coated dishes in stem cell maintenance media containing 10 μM ROCK inhibitor (Y-27632). The next day, media was changed to a neural induction media (knockout DMEM with 15% KSR, L-glutamine, NEAA, 100 nM LDN193189 (LDN, Stemgent) and 10 μM SB431542 (SB, Tocris)), which represents day 0 of differentiation. The KSR component is gradually reduced and replaced with increasing amounts of N2 media from day 4 to 10 as described previously⁴⁹. To promote the anterior forebrain fate, 2 μM of XAV939 (Stemgent) is added to the neural induction media from day 0 to 5⁵⁰. Between day 10-12 of differentiation, cells were either used to generate a.) rosette-stage NSCs or b.) LTNSCs.

a.) Rosette-stage NSC generation. Differentiated cells were dissociated with Accutase for 30 minutes and cells were passed through a 0.45-micron cell strainer and pelleted. Cells were then resuspended in N2 media containing 10 ng/ml Brain Derived Neurotrophic Factor (BDNF), 10 mM Ascorbic Acid (AA) and 1 ng/ml Sonic Hedgehog (SHH) at a concentration of 4×10⁵ cells/μl. 20 μl droplets are made on dried PO/Lam/FN plates. After the cells have settled, the remaining dish is filled with the N2 media containing BDNF, AA and SHH. By 3-5 days of culture, neural rosette formation should be apparent.

b.) LTNSC generation. Differentiated cells were dissociated using 10% Dispase for 10 minutes. Cells were then separated as clumps and resuspended in N2 media containing 20 ng/ml FGF2 and cultured in sterile, non-TC treated dishes. Cells should form a high number of neurospheres and by 3-5 days neural rosette formation within the spheres should be apparent. Once the neurosphere cultures are pure, they are landed on PO/Lam/FN plates and cultured in N2 with 10 ng/ml FGF2, 10 ng/ml EGF and 1:1000 B27 supplement (NSC media). Rosette-stage NSC outgrowth is observed until confluency and then passaged at high density (roughly a 1:3 passage) over 2-3 months. Cells maintaining a neuroepithelial morphology by passage 10 in NSC media were kept and analyzed for early NSC markers and differentiation potential.

### Spinal Motor Neuron (SMN) Induction Protocol

Human pluripotent stem cells were differentiated toward the spinal cord fate as described previously²³. Briefly, induced pluripotent cells were seeded on matrigel coated dishes and differentiated with LDN, SB and Retinoic Acid (1 μg/ml) for 10-12 days. Cells were dissociated and cultured in N2 media including BDNF, AA and glial derived neurotrophic factor (GDNF) for 14 days.

### Astrocyte Induction Protocol

Typically, regionally patterned neural stem cells are infected with lentiviral particles containing FUW-NFIA and FUW-M2-rtTA and induced with doxycycline (dox) one day after infection. Cells are kept in dox media for a minimum of 5 days and then switched to the astrocyte induction media (N2 media with 10 ng/ml HB-EGF (R&D Systems) and 10 ng/ml Leukemia inhibitory factor (Peprotech)) without dox for a minimum of 5 days. Glial progenitors and astrocytes can be isolated using CD44.

### Immunohistochemistry and CD44 FACS Analysis

For immunohistochemistry, cells were fixed in PBS containing 4% paraformaldehyde (EMS) for 10 minutes and permeabilized using PBS with 0.5% Triton-X for 5 minutes and stored in PBS with 0.2% Tween-20. The blocking solution contained 5% donkey serum in PBS with 0.2% Tween-20. Primary antibodies were diluted in the blocking solution and typically incubated overnight at 4 C. Secondary antibodies conjugated to Alexa 488, Alexa 555 or Alexa 647 (Thermo) were added to the cells and incubated for 30 minutes. Nuclei were identified by staining the cells with 4′,6-diamidino-2-phenylindole (DAPI, Thermo). A list of antibodies used in this study is presented in Table 1.

TABLE 1
List of Antibodies Used

| | | Catalog | Host | Clone | Validated by | |
|---|---|---|---|---|---|---|
| Antigen | Supplier | Number | Species | name | Manufacturer | Assay |
| NFIA | Sigma | HPA006111 | Rabbit | Polyclonal | human | Immunofluorescence |
| | | | | | validated | |
| GFAP | Biolegend | 829401 | Chicken | Polyclonal | human | Immunofluorescence |
| | | | | | validated | |
| GFAP | Dako | Z033429-2 | Rabbit | Polyclonal | human | Immunofluorescence |
| | | | | | validated | |
| GFP | Abcam | ab13970 | Chicken | Polyclonal | yes | Immunofluorescence |
| SLC1A2 | Abcam | ab416 | Rabbit | Polyclonal | human | Immunofluorescence |
| | | | | | validated | |
| SYN1 | Sigma | S193 | Rabbit | Polyclonal | human | Immunofluorescence |
| | | | | | validated | and Western Blot |
| MUNC13.1 | Synaptic | 126103 | Rabbit | Polyclonal | human | Western Blot |
| | Systems | | | | validated | |
| human | Takara | Y40410 | Mouse | unknown | human | Immunofluorescence |
| cytoplasm | | | | | validated | |
| human GFAP | Takara | Y40420 | Mouse | unknown | human | Immunofluorescence |
| | | | | | validated | |
| TUJ1 | R&D | MAB1195 | Mouse | TuJ-1 | human | Immunofluorescence |
| | | | | | validated | |
| CCNA1 | Santa Cruz | sc-271645 | Goat | Polyclonal | human | Western Blot |
| | | | | | validated | |
| CDKN1A | Thermo | MA5-14949 | Mouse | R.229.6 | human | Western Blot |
| | | | | | validated | |
| GAPDH | Fitzgerald | 10R-G109A | Mouse | 6C5 | human | Western Blot |
| | | | | | validated | |
| POU5F1/OCT4 | Cell | 2750S | Rabbit | Polyclonal | human | Immunofluorescence |
| | Signaling | | | | validated | |
| | Technology | | | | | |
| SOX2 | Biolegend | 630802 | Rabbit | Polyclonal | human | Immunofluorescence |
| | | | | | validated | |
| PLZF | R&D | MAB2944 | Mouse | 6318100 | human | Immunofluorescence |
| | | | | | validated | |
| AQP4 | Santa Cruz | sc-9888 | Goat | Polyclonal | tested in | Immunofluorescence |
| | | | | | mouse | |
| MAP2 | Sigma | M1406 | Mouse | AP-20 | human | Immunofluorescence |
| | | | | | validated | |
| NESTIN | Neuromics | M015012 | Mouse | 196908 | human | Immunofluorescence |
| | | | | | validated | |
| OTX2 | Neuromics | GT15095 | Goat | Polyclonal | human | Immunofluorescence |
| | | | | | validated | |
| FOXG1 | Neumcell | NCFAB | Rabbit | Polyclonal | human | Immunofluorescence |
| | | | | | validated | |
| ZO-1 | BD | 610966 | Mouse | 1/Z0-1 | human | Immunofluorescence |
| | Biosciences | | | | validated | |
| CD44 | Cell | 3570 | Mouse | 156-3C11 | human | Immunofluorescence |
| | Signaling | | | | validated | |
| | Technology | | | | | |
| CD44 | Biolegend | 103018 | Mouse | IM7 | human | FACS |
| | | | | | validated | |

For FACS and analysis, cells were dissociated using 0.05% Trypsin and inactivated with serum containing media. Cells were then resuspended in a sort buffer (1×PBS with 1 mM EDTA and 2% FBS) and incubated with a CD44-Alexa 647 antibody (Biolegend) for 30 minutes on ice.

Cells were then Subjected to Sort or Analysis Using the Flowjo Software.

Generation of NFIA and SOX9 inducible constructs and lentiviral production NFIA and SOX9 were cloned from cDNA from hPSC-derived astroglial progenitors (d90). FUW-tetO-GFP (Addgene 30130) was digested with EcoRI to remove the GFP fragment and NFIA or SOX9 was inserted using traditional ligation cloning. Plasmids containing NFIA, SOX9, FUCCI-O or M2-rtTA (Addgene 20342), the psPAX2 (Addgene 12260) packaging vector and the pMD2.G (Addgene 12259) envelope was transfected into 293T cells using X-tremeGene HP (Sigma) in a 1:2:1 molar ratio, respectively. Virus was harvested at 48 and 72 hours post transfection and concentrated using AMICON Ultra-15 Centrifugal Filter Units (Millipore).

### Gene Expression and ATAC-Seq Analysis

RNA-Sequencing:

Raw FASTQ files were trimmed for adapters and aligned to the ENSEMBL GRCh38 genome build using STAR 2.5.0. Matrices were generated from the aligned files using HTSeq⁵¹ and imported into DESeq2⁵² for further analysis using a standard pipeline.

ATAC-Sequencing:

Raw FASTQ files were aligned to the hg19 genome build using Bowtie2⁵³. Comparative analysis of alignment files was performed using the deepTools software package⁵⁴. Motif analysis and peak annotation was performed using the HOMER software⁵⁵ and visualized using the IGV browser⁵⁶. All FASTQ files and supplemental files are uploaded to NCBI GEO under the accession GSE104232.

### Cell Cycle Analysis

Cells were dissociated nuclei were isolated for cell cycle analysis. Propidium Iodine (250 ng/ml) was added to the cells and analyzed by FACS. A minimum of 10,000 events was analyzed per condition. Data acquired was imported and analyzed by the Flowjo software.

Transplantation of NSCs, Glial Precursors and Astrocytes into Adult Cortex

All surgeries were performed according to the NIH guidelines, and were approved by the local Institutional Animal Care and Use Committee (IACUC), the Institutional Biosafety Committee (IBC) and the Embryonic Stem Cell Research Committee (ESCRO). A total of 8 (or 20) [CF 1] NOD-SCID IL2-Rgc null mice (20-35 g; Jackson Laboratory) received cell transplantation. Mice were anesthetized with isoflurane 5% and kipping maintenance flow of₂-3%. A total of 7×10⁴ NFIA-induced astrocytes in 2 l were transplanted through a 5 μl-Hamilton syringe at a rate of 1 l/min by an infusion pump attached to a stereotactic micromanipulator, into the genu of the corpus callosum (coordinates: AP+0.740, ML −1.00, DV −2.30 from bregma). A total of 2×10⁵ LTNSCs/2 l were transplanted into the subcortical gray matter, striatum (coordinates AP+0.500, ML −1.900, DV −3.200 from bregma). A total of 7.5×10⁴ H1 derived-Astrocytes GFP—in 2 l was transplanted into the genu of the corpus callosum (coordinates: AP+0.740, ML −1.00, DV-2.30 from bregma). The mice were sacrificed at 1, 6 and 12 weeks after transplantation for IHC analysis.

### Tissue Processing

Mice were euthanized with overdose of pentobarbital intraperitoneally and transcardially perfused with phosphate buffered saline then paraformaldehyde (PFA) 4%. Brains were removed after gentle dissection and kept in overnight 4% PFA then soaked in 30% sucrose for 2-3 days. Brain coronal sections of 30 μm thick at −20° C. were performed by cryostat after embedding the brain with O.C.T (Sakura Finetek).

### Calcium Imaging

hPSC-derived neural stem cells or astrocytes and primary astrocytes (Sciencell) were plated onto PO/Laminin/Fibronectin coated 0.5 mm black ΔT dishes (Bioptechs) and used for calcium imaging as previously described⁵⁷ between days 60 and 120 from hPSCs. Cultures were incubated with 5 uM of Fura-2 (Thermo) for 30 minutes at 37 C and dishes were mounted to a ΔT Heated Lid w/Perfusion system (Bioptechs). Cultures were perfused with normal Tyrode's solution (pH 7.4) containing 125 mM NaCl, 5 mM KCl, 25 mM Glucose, 25 mM HEPES, ImM MgCl₂, 2 mM CaCl₂ and 0.1% (w/v) bovine serum albumin. Cultures were supplemented with glutamate (100 μM), ATP (30 μM) or KCl (65 mM) for 1 minute and imaged every 30 seconds at 340 and 380 nm at a minimum of 7 positions. Time-lapse images were analyzed using FIJI (ImageJ) by calculating the signal ratio between 380/340 nm.

### Glutamate Excitotoxicity Assay

Cortical neurons were derived by differentiating hPSCs towards the neuroectodermal fate (see above). Neuroectodermal cells were then dissociated and replated to generate neural rosettes and further differentiated into neurons by the treatment with DAPT. Neurons were then replated and assayed for maturation markers or glutamate excitoxicity⁵⁸ with or without astrocytes. For glutamate excitotoxicity studies, 100,000 neurons/cm² were plated on PO/Laminin/Fibronectin dishes in N2 media with BDNF, AA, and GDNF. NFIA-induced astrocytes were added at 150,000 cells/cm² and co-cultured for an additional 5 days. Cells were then treated with 100 or 500 μm (final) of L-glutamate for 1 hour in HBSS and recovered in N2 media with BDNF, AA, and GDNF. Resazurin was added 48 hours after glutamate treatment to determine cell viability.

### Bisulfite Conversion and Sequencing

LTNSCs infected with NFIA were treated with dox for 5 days and sorted for CD44. Cells were isolated and bisulfite conversion was performed using the EZ DNA Methylation-Direct™ Kit (Zymo) as described by manufacturer. Primers to the regions ofDNMT1 STAT3 binding site was described previously⁵⁹. The DNMT1 promoter region was amplified using ZymoTaq Premix (Zymo) and cloned into the TOPO Zero Blunt vector (Invitrogen). A minimum of 10 colonies were sent for sequencing per condition.

Results

To date, the study of human astrocyte biology has been challenging due to their limited availability and regional heterogeneity⁵. Understanding early developmental events in human astrocyte biology is particularly difficult as there is currently no robust in vitro system to interrogate their role in development and disease. Human PSCs represent an ideal model system to gain insights into early human development and to provide access to defined cell types of the human body. A unique trait of directed hPSC differentiation is that the cells will first initiate an embryonic developmental program before eventually transitioning to the production of later, adult-like cells⁴. Directed differentiation of hPSCs into NSCs results in a long neurogenic phase followed by a late gliogenic switch, mimicking the time-line of human development. Previous studies report the need to culture hPSC-derived NSCs for up to 24 weeks before obtaining large populations of functional astrocytes upon differentiation^{6,7}. Following such extended culture, the gliogenic switch occurs spontaneously, but the molecular mechanism underlying the switch remains unclear⁸.

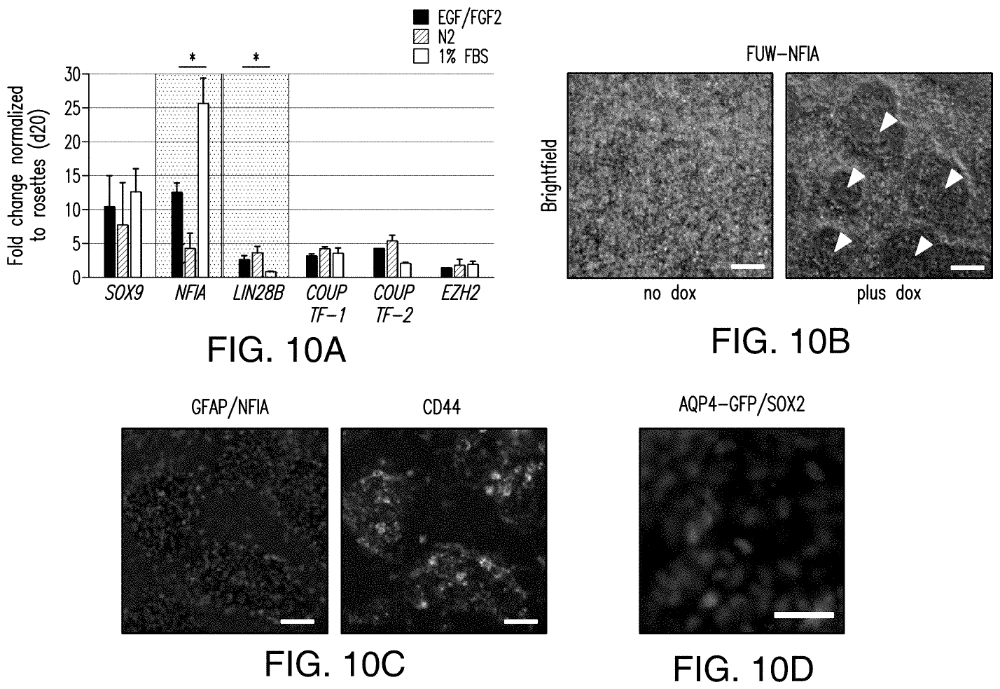

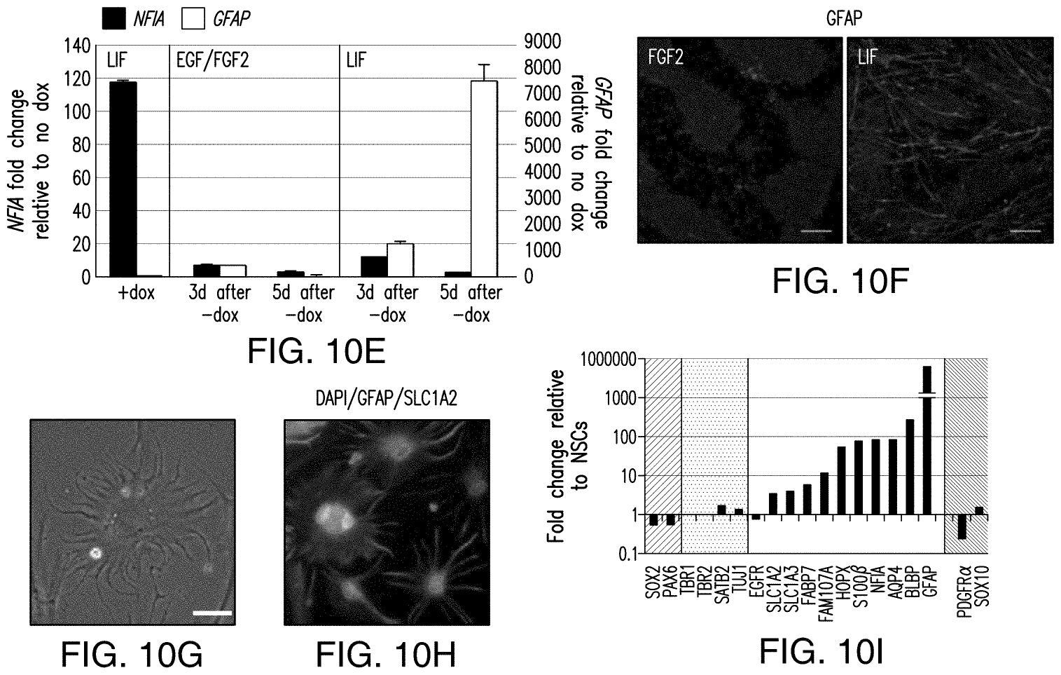

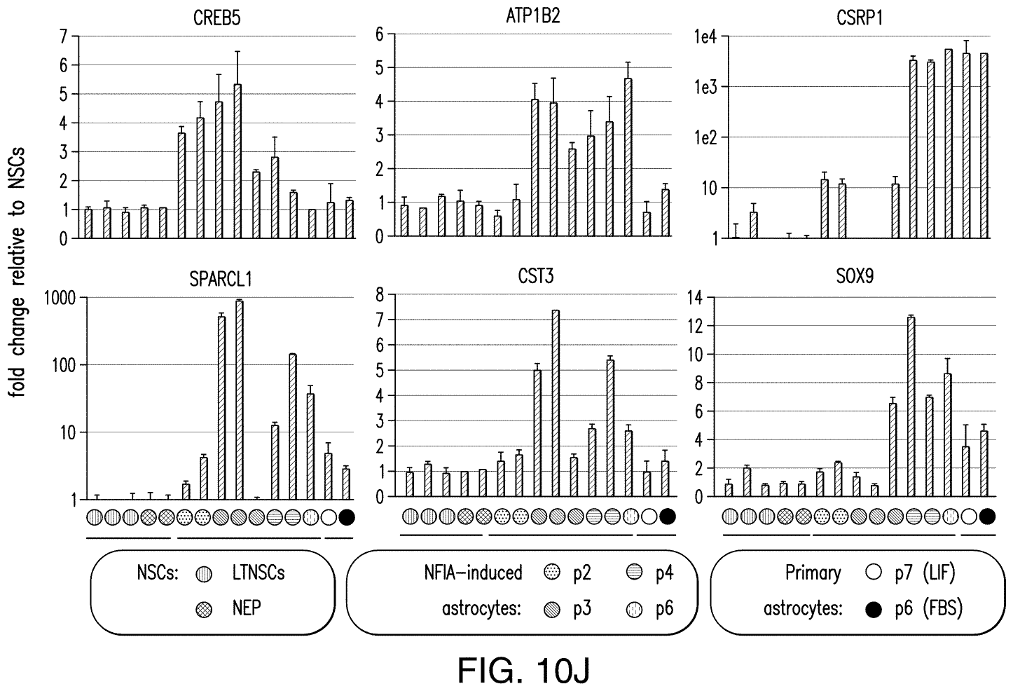

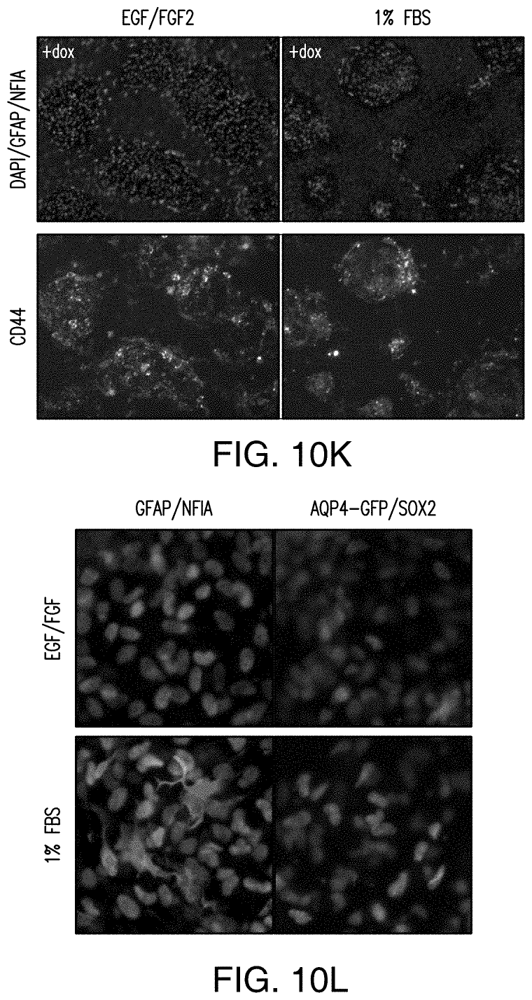

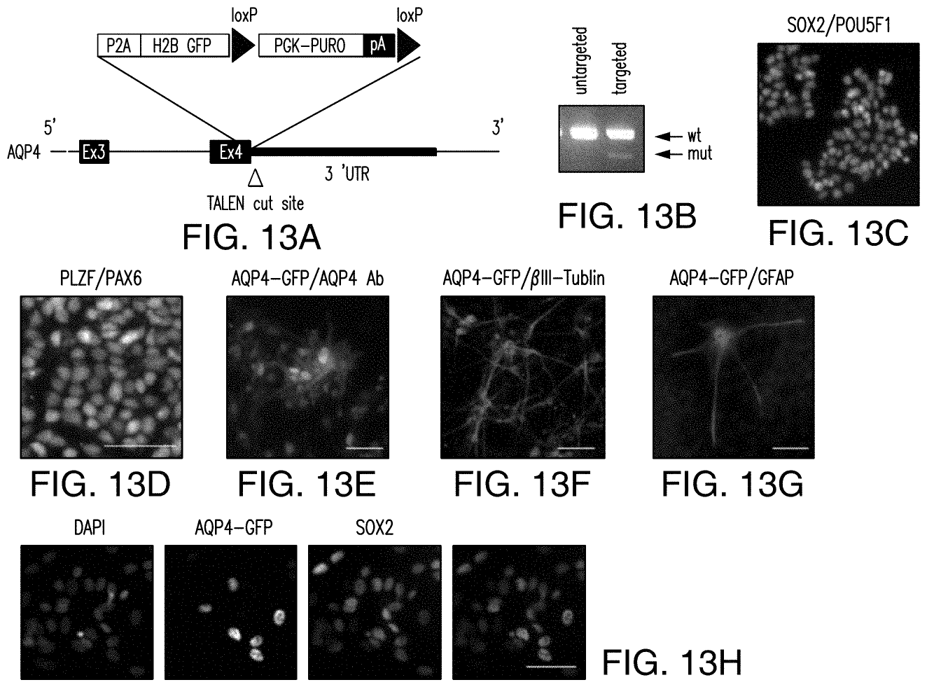

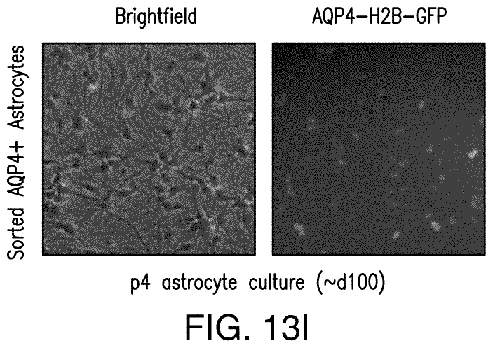

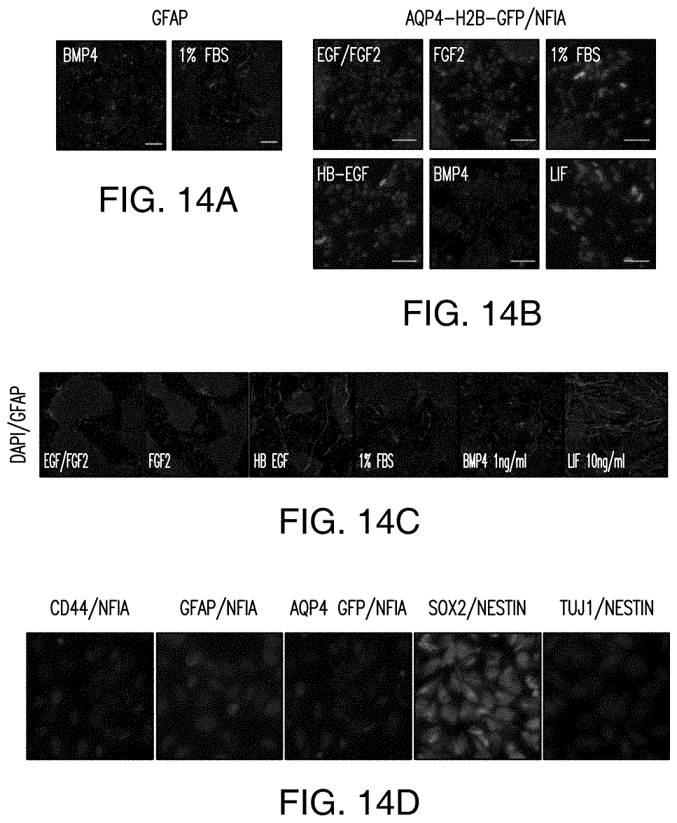

To precisely monitor when astrocytes develop during hPSC differentiation, a knock-in reporter line targeting the aquaporin-4 (AQP4) locus with a nuclear green fluorescent protein (H2B-GFP) was generated (FIGS. 13A-13H). Previous strategies for generating astrocytes from hPSCs include the exposure of factors such as LIF, CNTF, BMP, or serum to NSCs to trigger glial differentiation^{13,14}. The onset of glial differentiation was moderately accelerated in NSCs treated with serum (FIGS. 3A-3B) and correlated with significant changes in the expression of NFIA and LIN28B (FIG. 10A), factors previously implicated in glial fate acquistion^{15,16,17,18,19}. To directly test whether either gene impacts glial competency or differentiation, a homogeneous and stable neurogenic NSC population termed lt-hESNSCs²¹ (LTNSCs) was used that does not spontaneously undergo the gliogenic switch upon long-term culture (FIGS. 1A-1H). While knockdown of LIN28B did not show any obvious effect, overexpression of NFIA profoundly altered LTNSC morphology (FIG. 10B) and correlated with expression of NFIA protein and CD44²², a marker of glial competency, though it did not result in GFAP positive cells (FIG. 10C). Interestingly, a subset of the NFIA-expressing cells activated the AQP4-H2B-GFP reporter (FIG. 10D). Based on these results, it was hypothesized that the overexpression of NFIA triggers glial competency but blocks glial differentiation. A time course study was performed in which LTNSCs were cultured in the presence (dox+) or absence (dox−) of forced NFIA expression. After 5 days, cells were switched to (dox−) either in a glial promoting condition (+LIF) or in NSC maintenance medium (+EGF/FGF2). The removal of doxycycline led to a gradual decline in NFIA expression for both conditions, but GFAP expression was strongly induced only in the (+LIF) group and only after NFIA was sufficiently downregulated (FIG. 10E). Interestingly, continued expression of NFIA (dox+) prevented LTNSCs from expressing GFAP even in the presence of LIF (FIG. 10E (dox+)). Taken together, glial competency can be achieved in 5 days by transient expression of NFIA compared to 90-180 days using current protocols (FIGS. 8A-8B).

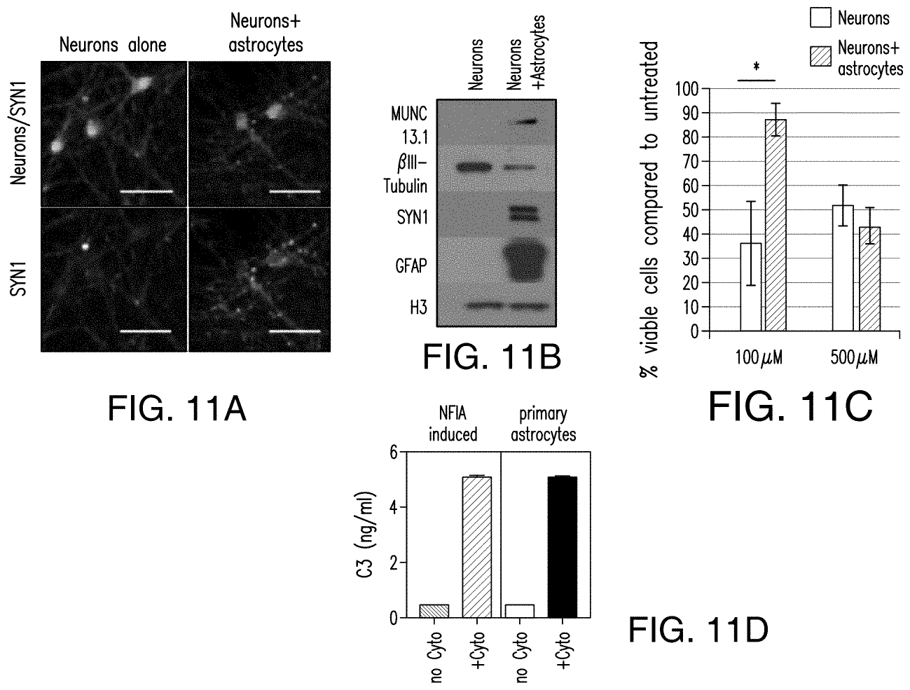

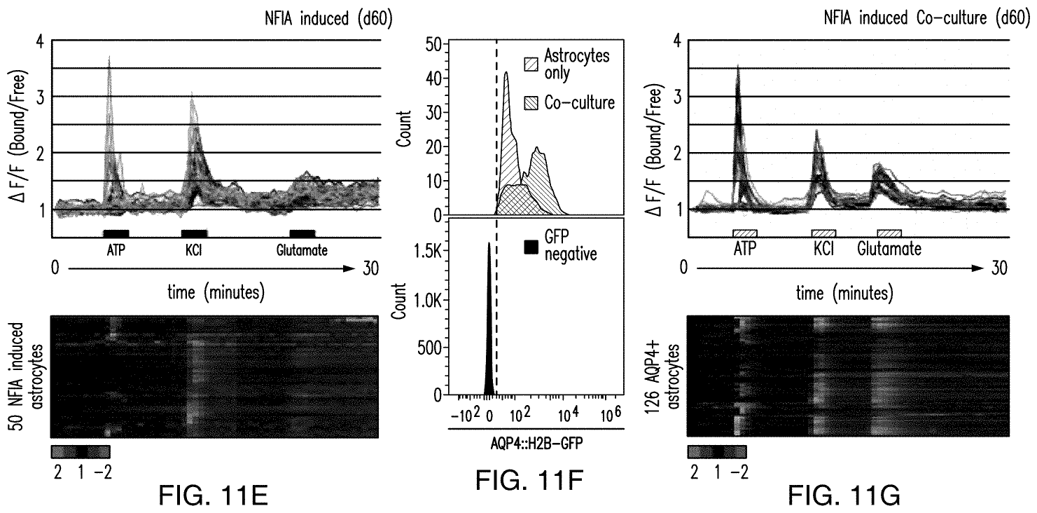

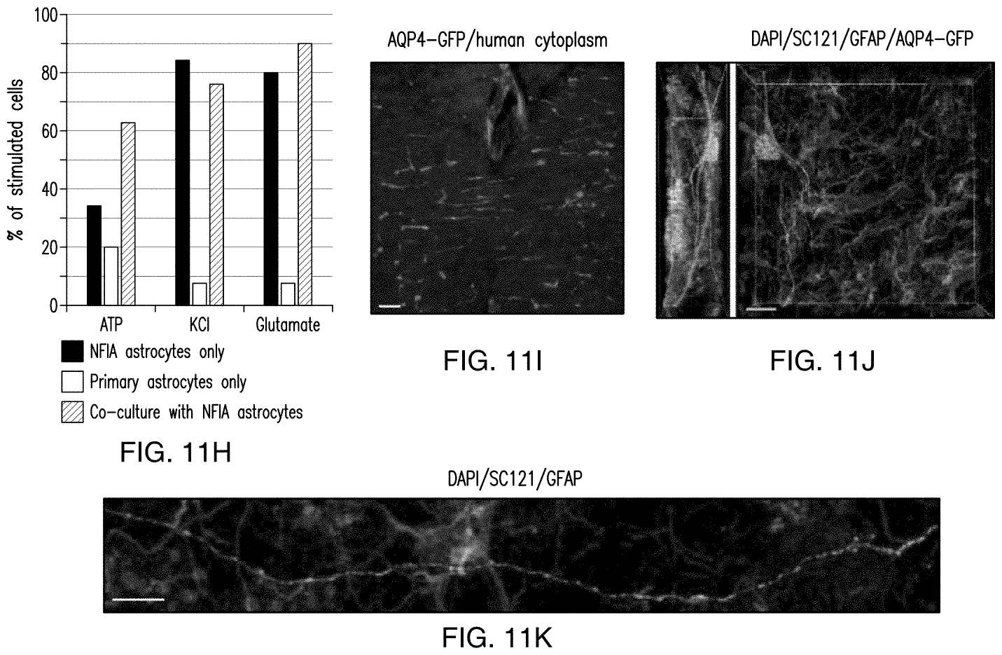

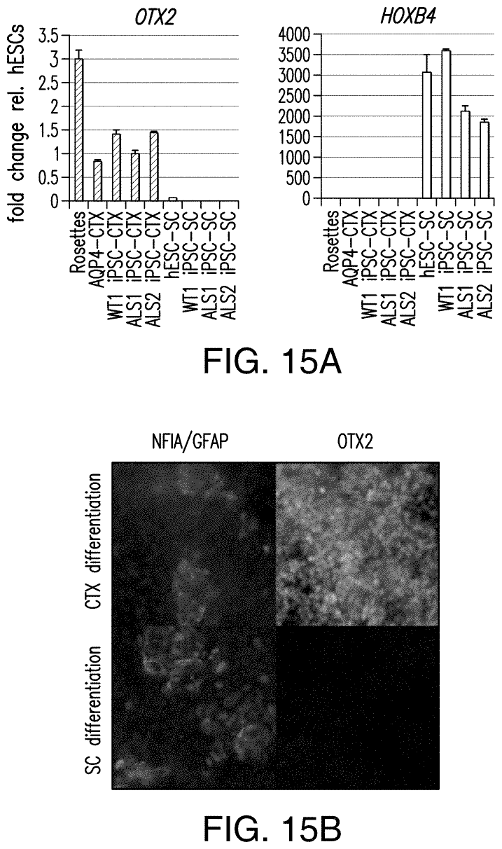

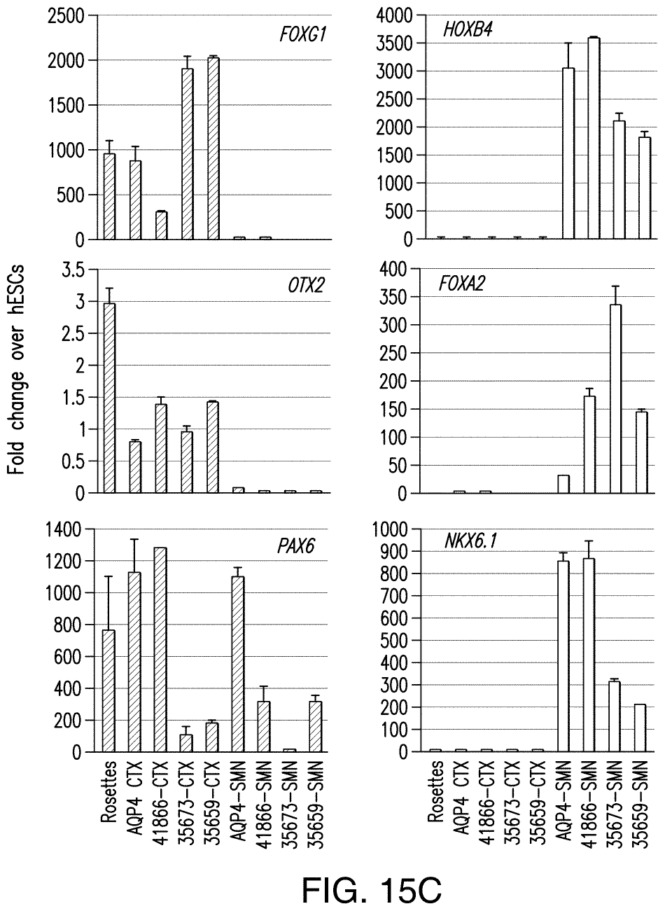

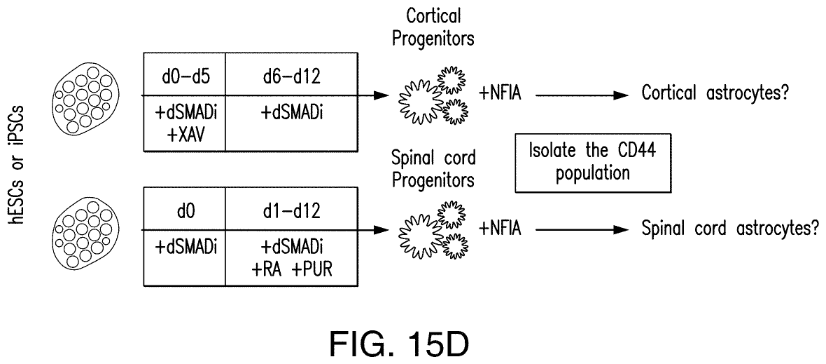

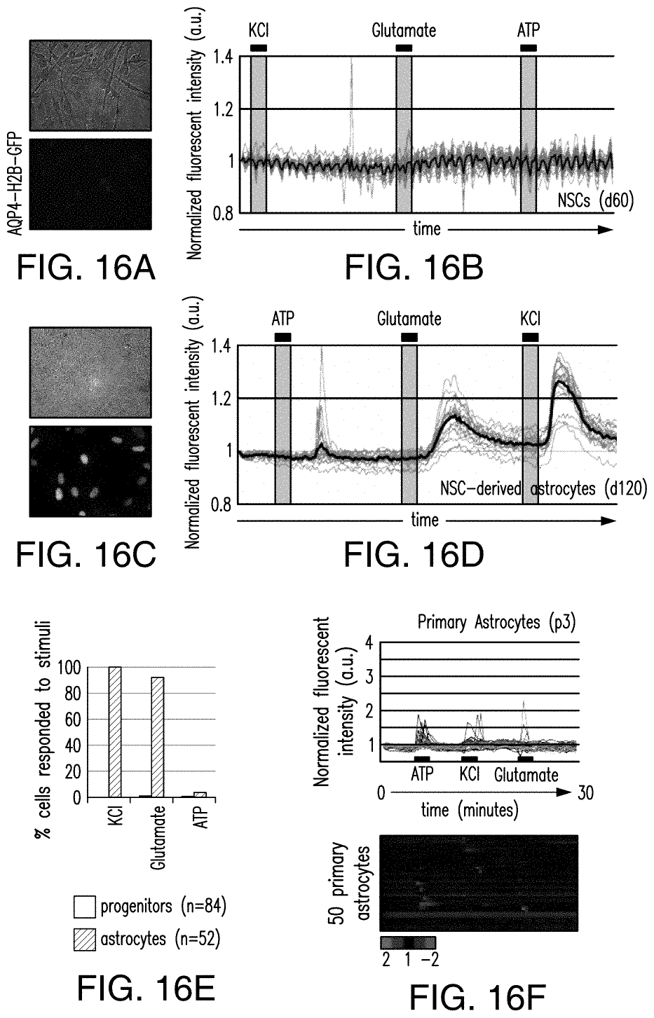

Next, it was analyzed whether other glial differentiation factors such as BMP4 or FBS promoted astrocyte differentiation of LTNSCs even more efficiently than LIF after dox removal. However, LIF was most efficient in generating GFAP+ cells and activating the AQP4-H2B-GFP reporter (FIG. 1F, FIG. 5). Upon further culture, the cells derived from NFIA-induced NSCs resembled astrocytes with complex morphologies (FIG. 1G) and expressed GFAP and SLC1A2 (FIG. 10H) as well as a panel of genes specifically associated with astrocyte identity (FIG. 10I). It was then examined the developmental stages of NFIA-induced astrocytes in comparison to primary human astrocytes⁵. NFIA-induced NSCs move through a fetal-like astrocyte program marked by the expression of CREB5, SPARCL1, A TPIB2 and CST3, towards an adult-like pattern marked by the expression of CSRP1 and SOX9, upon prolonged culture (FIG. 10J). Finally, patterning strategies was combined for establishing NSCs of distinct regional identities^{20,23} with NFIA expression to generate region-specific astrocytes of forebrain or spinal cord identity (FIGS. 15A-15B). The functionality of NFIA-induced astrocytes was then examined. Astrocytes play critical roles during CNS development including neuronal maturation²⁴, maintenance of metabolic homeostasis, and regulation of inflammation in the nervous system, among others²⁵. It was observed that immature neurons co-cultured in the presence of NFIA-induced astrocytes showed evidence of accelerated maturation by the punctate expression of SYN1 (synapsin-I) (FIG. 11A) and the increased expression of SYN1 and active zone marker MUNC13.1²⁶ (FIG. 11B). NFIA-induced astrocytes also promoted neuronal viability when subjected to glutamate excitotoxicity (FIG. 11C)²⁷. Importantly, upon cytokine treatment, reactive traits were readily trigger in NFIA-induced astrocytes such as complement (C3) secretion¹¹ (FIG. 11D). Astrocytes can also be stimulated to elicit calcium transients²⁸ in response to specific stimuli. Commercially available primary astrocytes isolated from human fetal brains (19-23 pcw) displayed morphologies similar to NFIA-induced astrocytes; however, very few cells responded to the stimuli (FIG. 16F). In contrast, NFIA-induced astrocytes responded robustly to KCl and ATP (FIG. 11E). When in co-culture with hPSC-derived neurons, NFIA-induced astrocytes showed increased AQP4-H2B-GFP signal (FIG. 11F) and the level of response to ATP increased by 2-fold (FIGS. 11G-11H). In addition, the magnitude of the glutamate response was enhanced, suggesting a synergistic interaction between the two cell types in driving both glial and neuronal maturation.

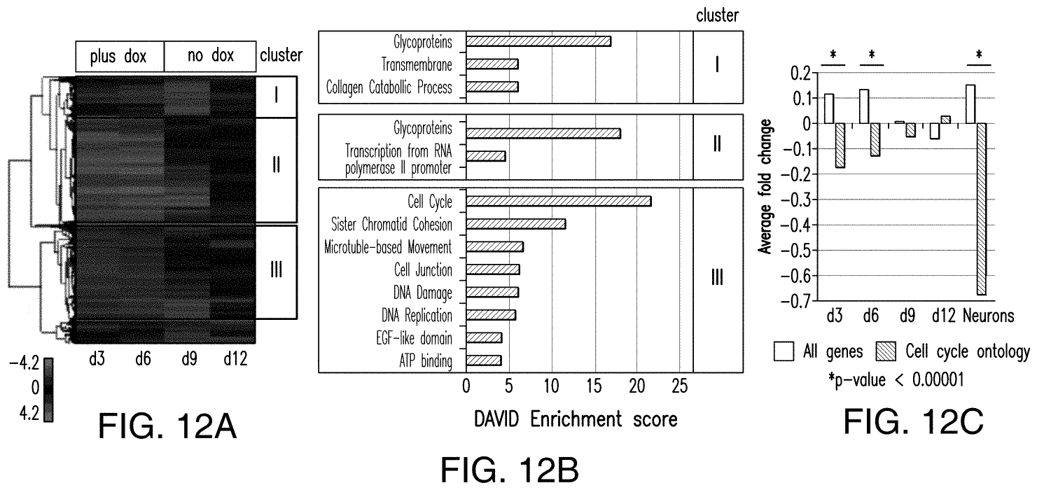

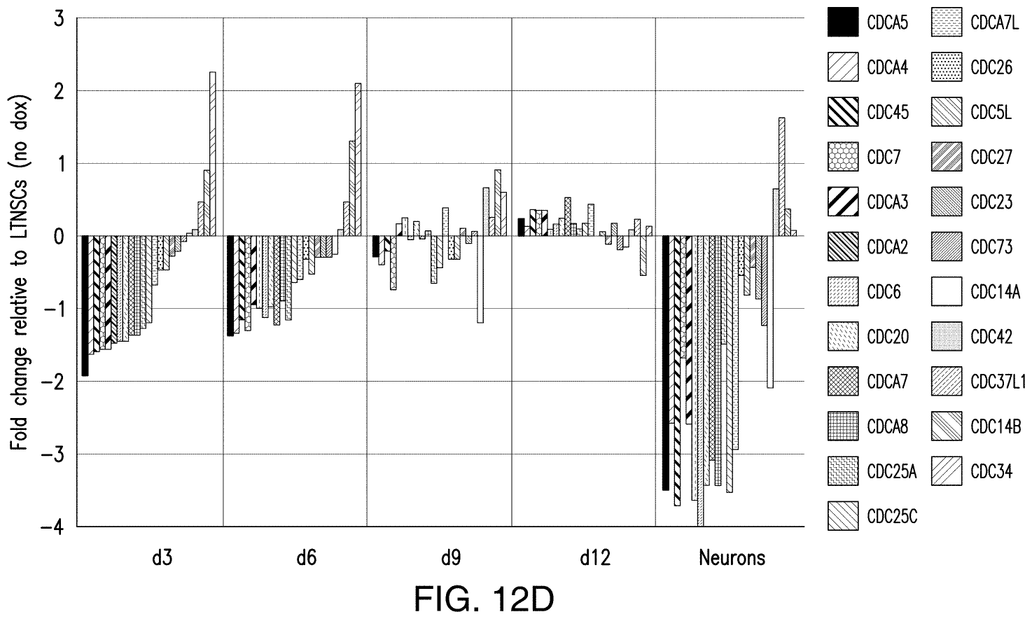

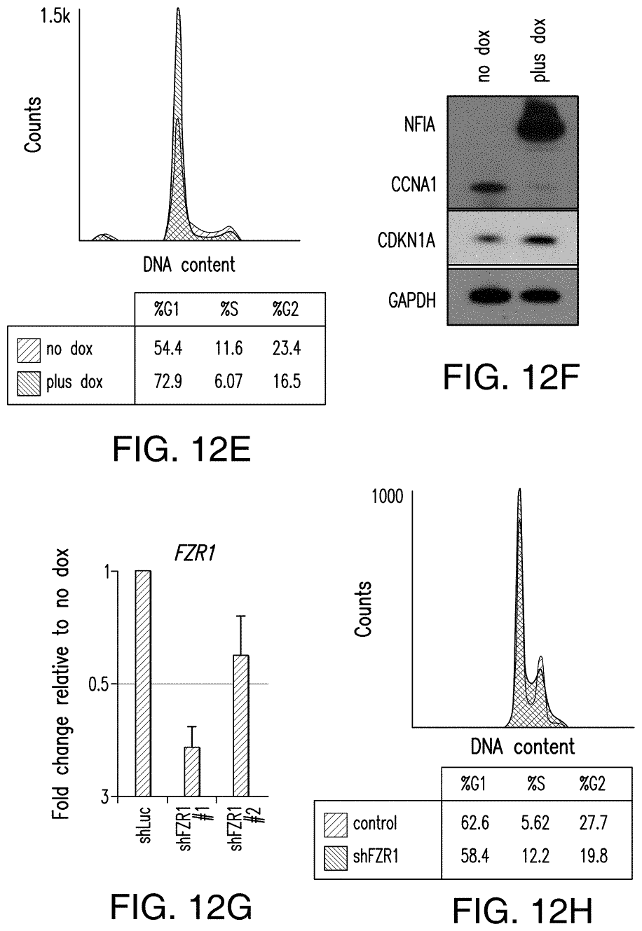

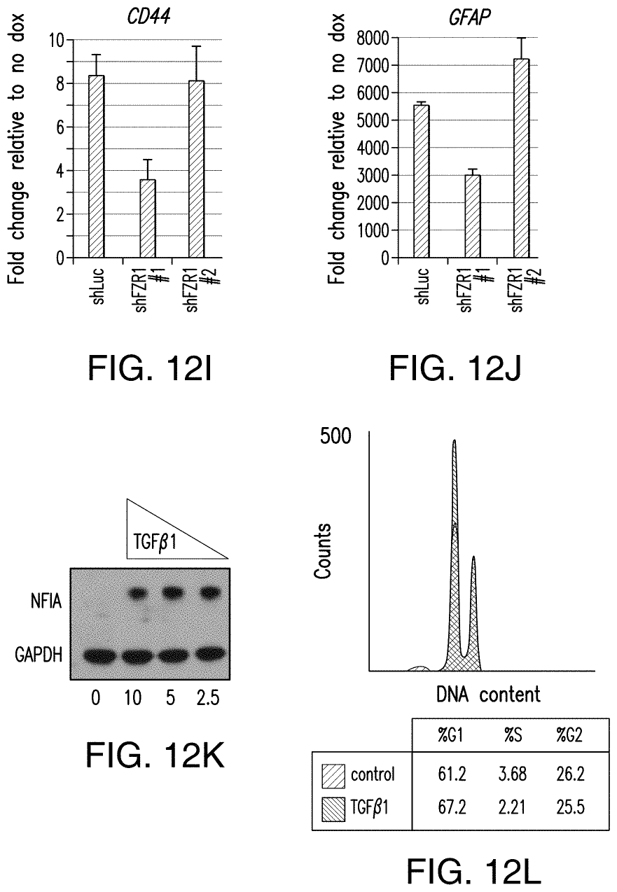

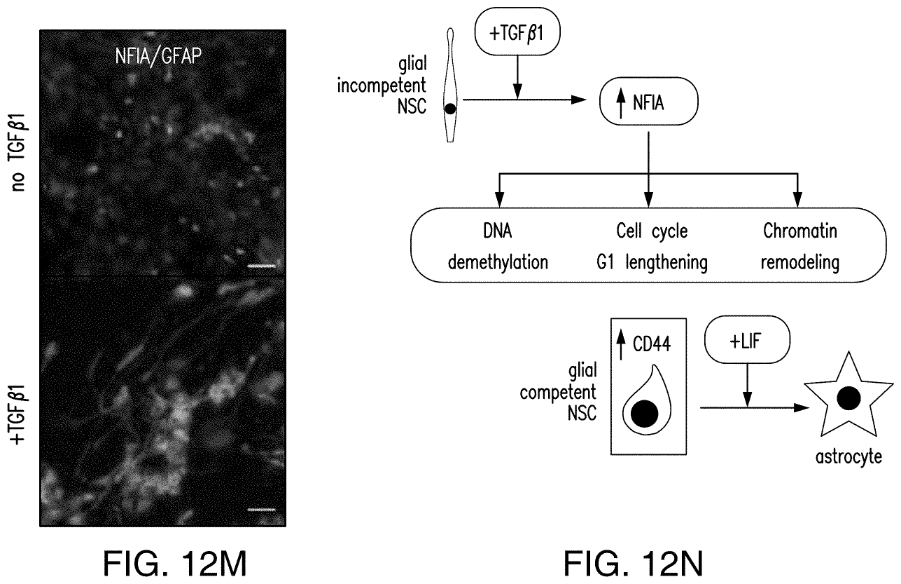

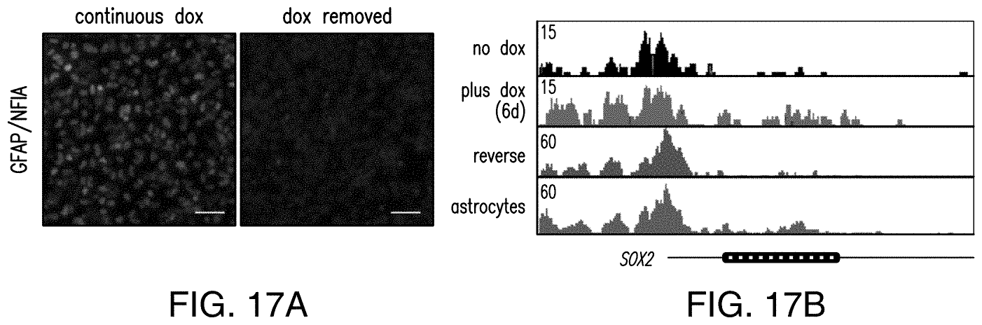

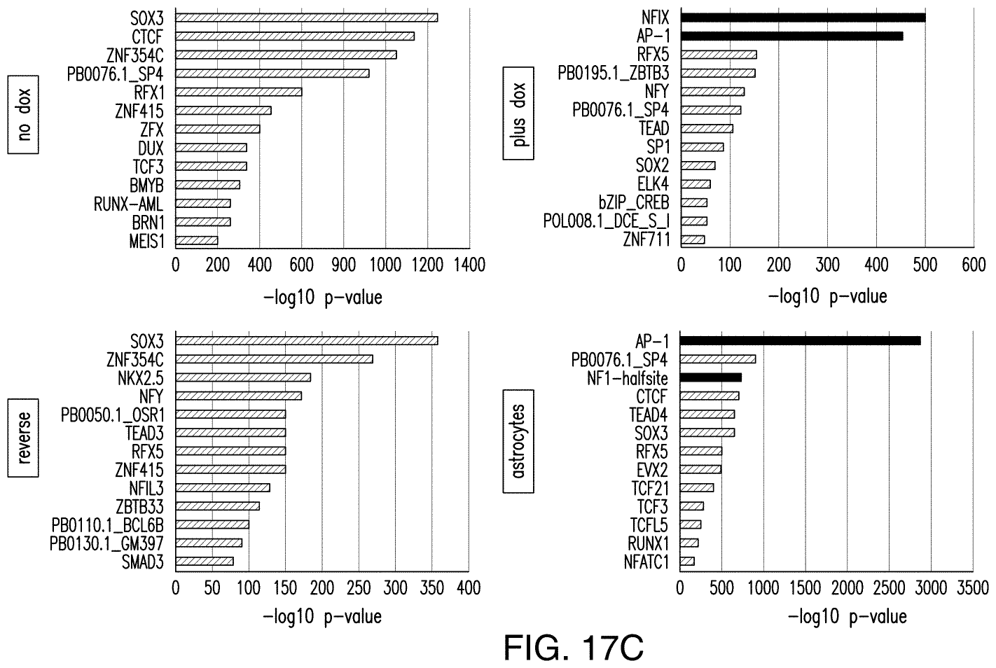

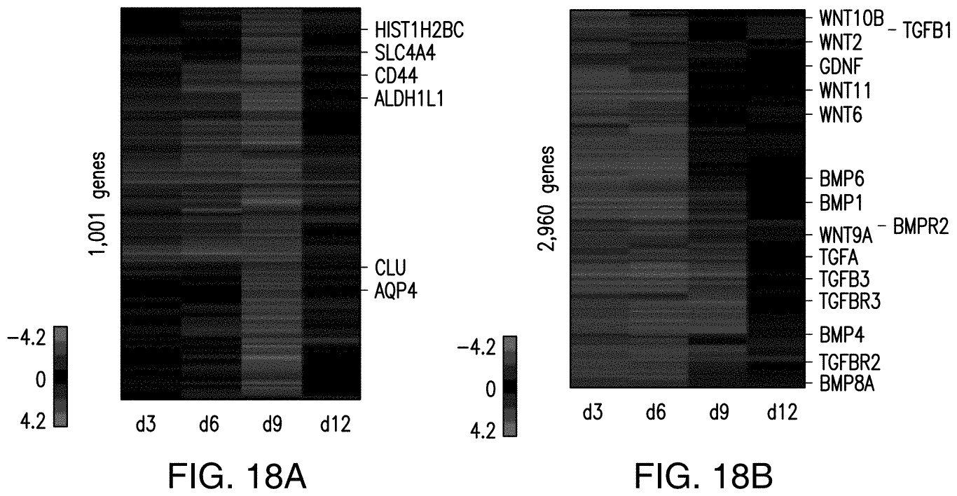

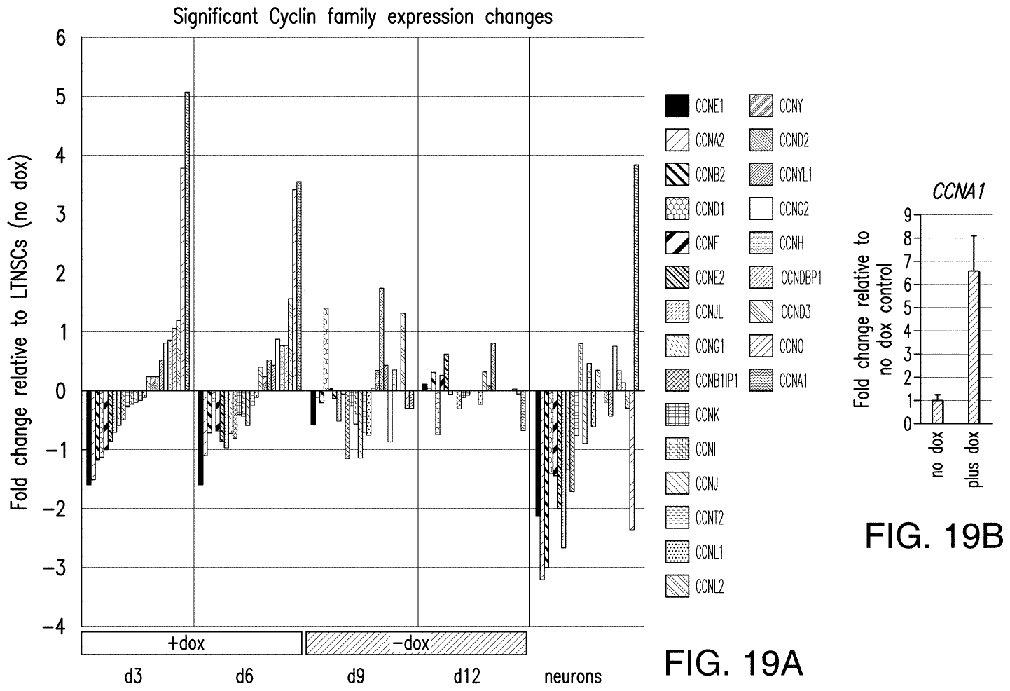

Finally, NFIA-induced glial competent NSCs and astrocytes were transplanted in the adult mouse cortex and corpus callosum. At 2 weeks post-transplantation, extensive migration of the glial progenitors from the graft core along white matter tracts was observed (FIG. 11I). The grafted cells maintained expression of AQP4-H2B-GFP as well as GFAP and displayed morphological features characteristic of human astrocytes by 6 weeks post-transplantation (FIG. 11J), such as long complex morphologies spanning multiple cortical regions²⁹. The above in vitro and in vivo data demonstrate that transient NFIA expression is a powerful strategy to generate functional, region-specific human astrocytes on demand and at unprecedented speed and efficiency. Furthermore, this results strongly suggest that NFIA represents the previously elusive molecular switch for triggering human glial competency. As a first step in exploring NFIA mechanism of action, it was returned to the initial observation that transient NFIA expression triggered glial competency but did not induce endogenous NFIA expression (refer to FIG. 10E). Once doxycycline was removed, cells progressively lost glial competency, including CD44 expression (FIG. 6A), and returned to a neurogenic state (FIG. 6B). A time course analysis of ectopic NFIA expression was performed followed by sorting for CD44 positive cells and replating in the absence of dox (dox-) (FIG. 6C). It was confirmed that NFIA expression was lost after 3 days of culture without doxycycline (d9) (FIG. 6D). It was also observed three major clusters among the samples throughout the time course (FIG. 6E). Of these, neuroepithelial stage NSCs, LTNSCs (dO), and samples reverted to dox-clustered together, supporting the notion that the NFIA pulse could not maintain glial competency upon NFIA withdrawal. Importantly, NFIA expression for 6 days induced a chromatin accessibility landscape similar to that of hPSC-derived astrocytes (d200) or glial competent NSCs (d80) (FIG. 6F). There was a clear shift in the enrichment of transcription factor binding motifs, where SOX and ZNF354C motifs were enriched in the dO (dox-) and the d12 (reverse) conditions, and AP-1, NFIX and NFI half site motifs were highly enriched in the d6 (dox+) and astrocyte conditions (FIGS. 17A-17C). Surprisingly, the GFAP promoter did not show differential accessibility (FIG. 6G, bottom), though the previous experiments showed robust induction of GFAP in the presence of LIF after only a short pulse of NFIA but not upon LIF treatment alone. However, upon bisulfite sequencing, one CpG in the GFAP promoter consistently showed a loss of methylation in NFIA-induced CD44+ cells (FIG. 6H) and was similarly unmethylated in astrocytes but not in LTNSCs or other rosette-stage NSCs. Importantly, the CpG matches a STAT3 binding site³¹ that is predicted to inhibit STAT3 binding when methylated. These data suggest that NFIA exerts multiple modes of inducing glial competency including the regulation of chromatin accessibility and DNA demethylation. It was next focused on the differential gene expression programs induced by NFIA overexpression and after NFIA withdrawal in LTNSCs using RNA sequencing and DAVID functional annotation analysis software³². Hierarchical clustering of all differentially expressed genes during the time course displayed three major clusters with distinct temporal profiles (FIG. 12A, labeled I, II and III). Cluster I (1,001 genes) includes genes associated with glial differentiation such as CD44 and AQP4. Additional genes associated with astrocyte identity—ALDHIL1, SLC4A4 and CLU⁵—were also detected in this cluster (FIG. 18A). Cluster II (2,960 genes) is comprised of genes directly affected by NFIA expression and which are rapidly lost upon NFIA reduction. This includes several WNT, TGFβ and BMP family members as well as trophic factors such as glial derived neurotrophic factor (GDNF) (FIG. 18B). Cluster III (2,593 genes) encompasses genes downregulated upon expression of NFIA. Remarkably, cluster III genes were specifically enriched for cell cycle related processes such as cell division, chromosome segregation, DNA repair and replication (FIG. 12B). Interestingly, NFIA triggered a negative regulation of cell cycle specific genes (FIG. 12C), which was reversible after NFIA removal (FIG. 12D, FIG. 19A).

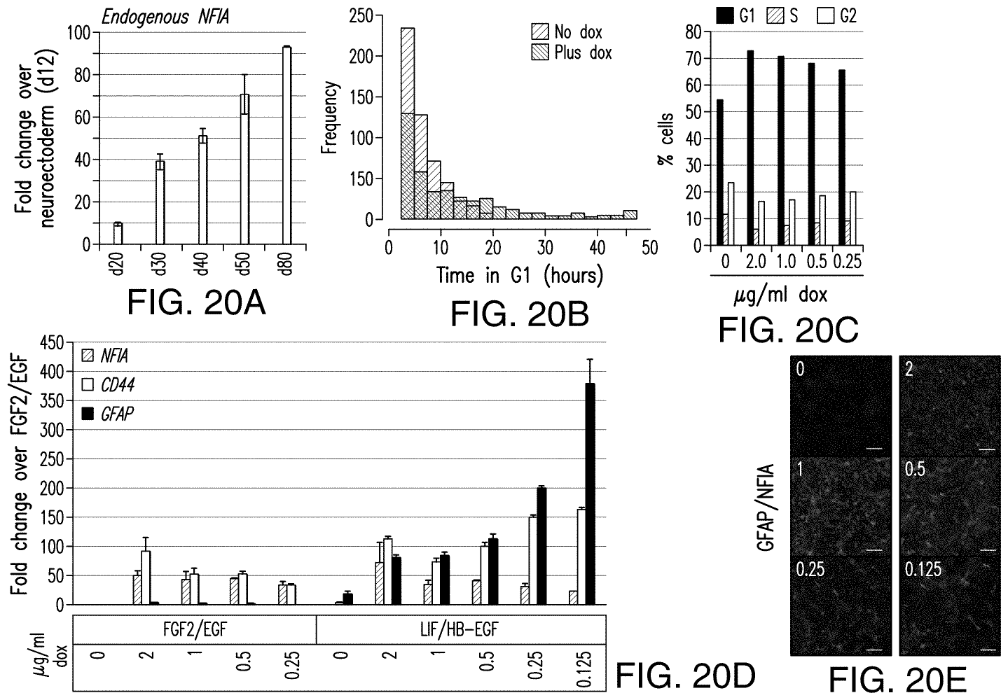

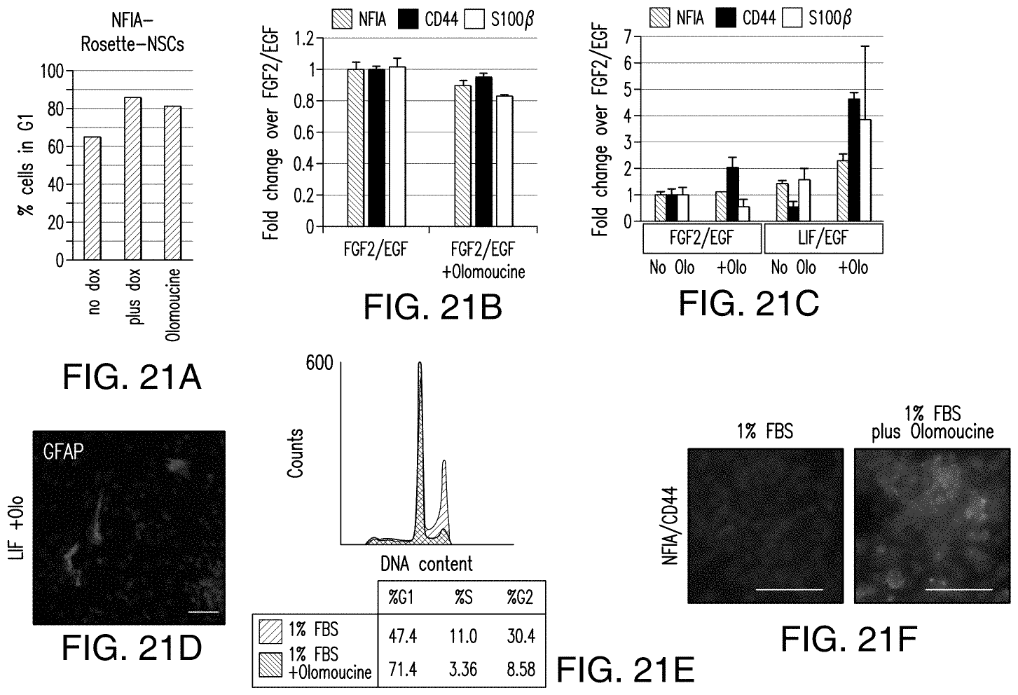

It was next investigated whether NFIA causes functional changes in cell cycle progression which could be key for the acquisition of glial competency. Interestingly, it was observed that a large proportion of cells accumulated in G1 following NFIA expression in LTNSCs (FIG. 12E). Although expression of CCNA1 was upregulated with NFIA (FIG. 19B), it was observed a striking decrease in CCNA1 protein and marked increase in CDKN1A (p21) (FIG. 12F), additional evidence that NFIA is maintaining NSCs in the G1 phase. Progressive lengthening of G1 has been previously reported as a characteristic feature of the developing rodent cortex³⁴ from early embryonic (neurogenic) to later fetal (gliogenic) stages—a transition during which hPSC-derived NSCs show progressive endogenous expression of NFIA (FIG. 20A). To directly measure cell cycle lengthening, FUCCI-O vector was used to determine the time cells spent in G1³⁵. Time-lapse analysis demonstrated that a large proportion of cells showed lengthening of the G1 phase in the (dox+) condition compared to uninduced cells (FIG. 20B). It was next investigated the reason continuous NFIA overexpression blocks astrocyte differentiation is due to a complete G1 arrest preventing cell cycle progression, and whether reducing the levels of NFIA would result in a more moderate G1 length compatible with glial fate acquisition. Indeed, when NFIA levels were titrated by varying dox-concentrations, the percentage of cells in G1 gradually decreased correlating with reduced dox levels, and GFAP expression was induced only at lower dox concentrations in the presence of LIF (FIGS. 20D 20E). To examine whether pharmacological modulation of the cell cycle alone in the absence of NFIA expression is sufficient to trigger the gliogenic switch, cells were treated with Olomoucine (Olo), a small molecule known to lengthen G1 timing in vitrO³⁶. Treatment of LTNSCs with Olo triggered an increase in the percentage of cells in G1 (FIG. 21A) but did not immediately increase the expression of glial competency genes (FIG. 21B). However, when Olo-treated LTNSCs were either maintained or induced to differentiate for an additional 12 days, an increased expression of NFIA, CD44 and S100 were detected, and remarkably detected GFAP positive cells (FIGS. 21C, 21D). Conversely, it was tested for the necessity of G1 lengthening during NFIA-mediated induction of glial competency by knocking down FZR1(CDH1), which previous studies have shown leads to a shortened G1 cell cycle phase^{37,38}. shRNA constructs targeting FZR1 showed efficient knockdown of the transcript (FIG. 12G), did not impact levels of NFIA expression, and decreased the percentage of cells in G1 (FIG. 12H). Indeed, the knockdown of FZR1 partially prevented NFIA-mediated induction of CD44 and GFAP expression (FIGS. 121, 12J). Finally, it was explored the identity of candidate upstream activators of NFIA. Transforming growth factor beta (TGFβ) family signaling has been implicated in timing of cell fate decisions in the spinal cord³⁹ and is known to modulate G1 arrest in a variety of proliferating cell lines⁴⁰. TGFβ1 treatment of neurogenic NSCs induced the expression of NFIA (FIG. 12K) and enriched for cells in the G1 phase of the cell cycle (FIG. 12L). Neurogenic NSCs were treated with or without TGFβ followed by culture in LIF-containing medium for 2 weeks, which resulted in the appearance of GFAP+ cells (FIG. 12M). These results indicate that TGFβ1-mediated induction of NFIA and concomitant G1 lengthening are sufficient to trigger precocious gliogenesis. However, the resulting levels of NFIA expression, speed and efficiency did not match the results obtained with ectopic NFIA expression, suggesting that further investigation into additional factors may be required to fully substitute for forced NFIA expression. While it has become routine to model neurodevelopmental or neurodegenerative diseases^{41,42} with hPSC-derived neurons, the use of hPSC-derived astrocytes in such studies has remained very limited^{6,43}. This is due, at least in part, to the extremely protracted onset of the gliogenic switch in humans as compared to rodent cells which is recapitulated in vitro, and which makes such studies highly laborious, costly, and inefficient. This work presents a simple and effective strategy based on the use of a single factor to drive glial competency and astrocyte differentiation (FIG. 12N). The disclosed ability to combine overexpression of NFIA with patterning early stage NSCs enables the rapid derivation of region-specific astrocytes and will be of particular interest in studying the contribution of astrocytes to disorders affecting particular brain regions such as in Parkinson's disease, Alzheimer's disease, RTT-syndrome, ALS, or hepatic encephalopathy. Such region-specific astrocytes can be further harnessed for the study of distinct trophic support, as reported for primary astrocytes derived from discrete brain regions⁴⁴.

The potential concern in using NFIA to dramatically fast-forward human neural development is whether the resulting cell types match bona fide in vivo derived astrocytes or may represent an artefactual in vitro cell type. The inventors demonstrated robust functional features of NFIA-induced astrocytes including calcium responses to relevant stimuli such as ATP, KCl or glutamate that not only match but exceed the performance of commercially available primary human fetal astrocytes. Therefore, the NFIA-protocol yields astrocyte populations highly relevant for human molecular, physiological and disease-related studies. Contrary to the role of NFIA overexpression in promoting competency for astrocyte differentiation, NFIA, at least when expressed at high levels, prevents further differentiation into astrocytes unless it is downregulated. It is possible that this finding explains the low efficiency of astrocyte induction in past ectopic expression studies in the chick spinal cord¹⁶, and that NFIA levels would need to be carefully titrated or followed by NFIA withdrawal to achieve optimal results. NFIA null mutant mice show a near complete loss of GFAP expression in the adult brain⁴⁵. A similar phenotype is observed in NFIB mutant mice⁴⁶. The likely redundancy of NFIA and NFIB in vivo may explain why single mutant mice do not exhibit an even more severe early developmental glial specification phenotype². Future studies may also address whether NFIB can functionally substitute for NFIA in the gain-of-function studies in hPSCs to trigger glial competency. Another intriguing finding is that transient overexpression of NFIA is not sufficient to activate an irreversible, endogenous glial competency program. NFIA-induced NSCs that subsequently lose NFIA expression in the absence of any STAT or BMP signal activators revert transcriptionally and epigenetically back to their early neurogenic state. These findings indicate that NFIA may act in concert with other factors such as SOX9 during in vivo development to stabilize the gliogenic program.

The mechanistic studies demonstrate that NFIA can rapidly trigger a chromatin state similar to that of astrocytes. Similarly, the gene expression data reveal that NFIA induces transcription of a broad set of genes related to glial specification. Upon the release of NFIA overexpression several genes associated with astrocyte identity remain upregulated for at least an additional 3 days suggesting that NFIA may open the chromatin landscape at these particular genes to poise them for activation in response to extrinsic factors. Nuclear Factor 1B (NFIB), a homolog of NFIA, has been shown to drive metastasis in small lung cancers by increasing chromatin accessibility to allow activation of a metastatic program⁴⁷. In agreement with the role of NFIB, it was found the NFI-motif to be highly enriched in the accessible chromatin with NFIA expression. Other highly enriched motifs such as the AP-1/JunB will need further study to investigate their potential role in astrocyte differentiation or in maintaining the glial competent state.

A particularly exciting finding was the role of NFIA as a negative cell cycle regulator triggering a prolonged G1 phase. It was demonstrated that pharmacological or genetic modulation of the G1 phase can affect the expression of glial progenitor markers. The role of the cell cycle in modulating cell fate decisions in undifferentiated hPSC populations has been described previously⁴⁸ where hESCs skew their differentiation potential depending on the phase of the cell cycle. These data demonstrate that NFIA expression inhibits the cell cycle progression by extending the G1 phase, and that high NFIA levels may trigger G1 arrest. Withdrawal of NFIA would allow cells to re-enter the cell cycle and thereby create a G1 cell cycle length compatible with glial differentiation. Further exploration of the interaction among TGF, NFIA and other factors in establishing the coordinated, progressive modulation of the cell cycle that promotes the glial competent state during in vivo development will be particularly interesting.

### REFERENCES

1 Liddelow, S. A. et al. Neurotoxic reactive astrocytes are induced by activated microglia. Nature 541, 481-487, doi:10.1038/nature21029 (2017).
2 Molofsky, A. V. et al. Astrocytes and disease: a neurodevelopmental perspective. Genes Dev 26, 891-907, doi:10.1101/gad.188326.112 (2012).
3 Sauvageot, C. M. & Stiles, C. D. Molecular mechanisms controlling cortical gliogenesis. Current opinion in neurobiology 12, 244-249 (2002).
4 Studer, L., Vera, E. & Cornacchia, D. Programming and Reprogramming Cellular Age in the Era of Induced Pluripotency. Cell Stem Cell 16, 591-600, doi:10.1016/j.stem.2015.05.004 (2015).
5 Zhang, Y. et al. Purification and Characterization of Progenitor and Mature Human Astrocytes Reveals Transcriptional and Functional Differences with Mouse. Neuron 89, 37-53, doi:10.1016/j.neuron.2015.11.013 (2016).
6 Krencik, R. et al. Dysregulation of astrocyte extracellular signaling in Costello syndrome. Sci Transl Med 7, 286ra266, doi:10.1126/scitranslmed.aaa5645 (2015).
7 Tao, Y. & Zhang, S. C. Neural Subtype Specification from Human Pluripotent Stem Cells. Cell Stem Cell 19, 573-586, doi:10.1016/j.stem.2016.10.015 (2016).
8 Chandrasekaran, A., Avci, H. X., Leist, M., Kobolak, J. & Dinnyes, A. Astrocyte Differentiation of Human Pluripotent Stem Cells: New Tools for Neurological Disorder Research. Front Cell Neurosci 10, 215, doi:10.3389/fncel.2016.00215 (2016).
9 Cahoy, J. D. et al. A transcriptome database for astrocytes, neurons, and oligodendrocytes: a new resource for understanding brain development and function. J Neurosci 28, 264-278, doi:10.1523/JNEUROSCI. 4178-07.2008 (2008).
Papadopoulos, M. C. & Verkman, A. S. Aquaporin water channels in the nervous system. Nat Rev Neurosci 14, 265-277, doi:10.1038/nrn3468 (2013).
11 Liddelow, S. A. & Barres, B. A. Reactive Astrocytes: Production, Function, and Therapeutic Potential. Immunity 46, 957-967, doi:10.1016/j.immuni.2017.06.006 (2017).
12 Levitt, P. & Rakic, P. Immunoperoxidase localization of glial fibrillary acidic protein in radial glial cells and astrocytes of the developing rhesus monkey brain. J Comp Neurol 193, 815-840, doi:10.1002/cne.901930316 (1980).
13 Santos, R. et al. Differentiation of Inflammation-Responsive Astrocytes from Glial Progenitors Generated from Human Induced Pluripotent Stem Cells. Stem Cell Reports 8, 1757-1769, doi:10.1016/j.stemcr.2017.05.011 (2017).
14 Krencik, R., Weick, J. P., Liu, Y., Zhang, Z. J. & Zhang, S. C. Specification of transplantable astroglial subtypes from human pluripotent stem cells. Nat Biotechnol 29, 528-534, doi:10.1038/nbt.1877 (2011).
15 Stolt, C. C. et al. The Sox9 transcription factor determines glial fate choice in the developing spinal cord. Genes Dev 17, 1677-1689, doi:10.1101/gad.259003 (2003).
16 Deneen, B. et al. The transcription factor NFIA controls the onset of gliogenesis in the developing spinal cord. Neuron 52, 953-968, doi:10.1016/j.neuron.2006.11.019 (2006).
17 Patterson, M. et al. let-7 miRNAs can act through notch to regulate human gliogenesis. Stem Cell Reports 3, 758-773, doi:10.1016/j.stemcr.2014.08.015 (2014).
18 Naka, H., Nakamura, S., Shimazaki, T. & Okano, H. Requirement for COUP-TFI and II in the temporal specification of neural stem cells in CNS development. Nat Neurosci 11, 1014-1023, doi:10.1038/nn.2168 (2008).
19 Hirabayashi, Y. et al. Polycomb limits the neurogenic competence of neural precursor cells to promote astrogenic fate transition. Neuron 63, 600-613, doi:10.1016/j.neuron.2009.08.021 (2009).
Elkabetz, Y. et al. Human ES cell-derived neural rosettes reveal a functionally distinct early neural stem cell stage. Genes Dev 22, 152-165, doi:10.1101/gad.1616208 (2008).
21 Koch, P., Opitz, T., Steinbeck, J. A., Ladewig, J. & Brustle, O. A rosette-type, self-renewing human ES cell-derived neural stem cell with potential for in vitro instruction and synaptic integration. Proc Natl Acad Sci USA 106, 3225-3230, doi:10.1073/pnas.0808387106 (2009).
22 Liu, Y. et al. CD44 expression identifies astrocyte-restricted precursor cells. Dev Biol 276, 31-46, doi:10.1016/j.ydbio.2004.08.018 (2004).
23 Calder, E. L. et al. Retinoic Acid-Mediated Regulation of GLI3 Enables Efficient Motoneuron Derivation from Human ESCs in the Absence of Extrinsic SHH Activation. J Neurosci 35, 11462-11481, doi:10.1523/JNEUROSCI.3046-14.2015 (2015).
24 Ullian, E. M., Sapperstein, S. K., Christopherson, K. S. & Barres, B. A. Control of synapse number by glia. Science 291, 657-661, doi:10.1126/science.291.5504.657 (2001).
25 Sofroniew, M. V. Astrocyte barriers to neurotoxic inflammation. Nat Rev Neurosci 16, 249-263, doi:10.1038/nrn3898 (2015).
26 Betz, A. et al. Munc13-1 is a presynaptic phorbol ester receptor that enhances neurotransmitter release. Neuron 21, 123-136 (1998).
27 Sofroniew, M. V. & Vinters, H. V. Astrocytes: biology and pathology. Acta Neuropathol 119, 7-35, doi:10.1007/s00401-009-0619-8 (2010).
28 Cornell-Bell, A. H., Finkbeiner, S. M., Cooper, M. S. & Smith, S. J. Glutamate induces calcium waves in cultured astrocytes: long-range glial signaling. Science 247, 470-473 (1990).
29 Oberheim, N. A. et al. Uniquely hominid features of adult human astrocytes. J Neurosci 29, 3276-3287, doi:10.1523/JNEUROSCI.4707-08.2009 (2009).
30 Fan, G. et al. DNA methylation controls the timing of astrogliogenesis through regulation of JAK-STAT signaling. Development 132, 3345-3356, doi:10.1242/dev.01912 (2005).
31 Rajan, P. & McKay, R. D. Multiple routes to astrocytic differentiation in the CNS. J Neurosci 18, 3620-3629 (1998).
32 Huang da, W., Sherman, B. T. & Lempicki, R. A. Systematic and integrative analysis of large gene lists using DAVID bioinformatics resources. Nat Protoc 4, 44-57, doi:10.1038/nprot.2008.211 (2009).
33 Kang, P. et al. Sox9 and NFIA coordinate a transcriptional regulatory cascade during the initiation of gliogenesis. Neuron 74, 79-94, doi:10.1016/j.neuron.2012.01.024 (2012).
34 Takahashi, T., Nowakowski, R. S. & Caviness, V. S., Jr. The cell cycle of the pseudostratified ventricular epithelium of the embryonic murine cerebral wall. J Neurosci 15, 6046-6057 (1995).
Sakaue-Sawano, A. et al. Visualizing spatiotemporal dynamics of multicellular cell-cycle progression. Cell 132, 487-498, doi:10.1016/j.cell.2007.12.033 (2008).
36 Calegari, F. & Huttner, W. B. An inhibition of cyclin-dependent kinases that lengthens, but does not arrest, neuroepithelial cell cycle induces premature neurogenesis. J Cell Sci 116, 4947-4955, doi:10.1242/jcs.00825 (2003).
37 Vodermaier, H. C. APC/C and SCF: controlling each other and the cell cycle. Curr Biol 14, R787-796, doi:10.1016/j.cub.2004.09.020 (2004).
38 Sigl, R. et al. Loss of the mammalian APC/C activator FZR1 shortens G1 and lengthens S phase but has little effect on exit from mitosis. J Cell Sci 122, 4208-4217, doi:10.1242/jcs.054197 (2009).
39 Garcia-Campmany, L. & Marti, E. The TGFbeta intracellular effector Smad3 regulates neuronal differentiation and cell fate specification in the developing spinal cord. Development 134, 65-75, doi:10.1242/dev.02702 (2007).
40 Zhang, Y., Alexander, P. B. & Wang, X. F. TGF-beta Family Signaling in the Control of Cell Proliferation and Survival. Cold Spring Harb Perspect Biol 9, doi:10.1101/cshperspect.a022145 (2017).
41 Zeltner, N. & Studer, L. Pluripotent stem cell-based disease modeling: current hurdles and future promise. Curr Opin Cell Biol 37, 102-110, doi:10.1016/j.ceb.2015.10.008 (2015).
42 Sances, S. et al. Modeling ALS with motor neurons derived from human induced pluripotent stem cells. Nat Neurosci 19, 542-553, doi:10.1038/nn.4273 (2016).
43 Williams, E. C. et al. Mutant astrocytes differentiated from Rett syndrome patients-specific iPSCs have adverse effects on wild-type neurons. Hum Mol Genet 23, 2968-2980, doi:10.1093/hmg/ddu008 (2014).
44 Le Roux, P. D. & Reh, T. A. Astroglia demonstrate regional differences in their ability to maintain primary dendritic outgrowth from mouse cortical neurons in vitro. J Neurobiol 27, 97-112, doi:10.1002/neu.480270110 (1995).
45 das Neves, L. et al. Disruption of the murine nuclear factor I-A gene (Nfia) results in perinatal lethality, hydrocephalus, and agenesis of the corpus callosum. Proc Natl Acad Sci U SA 96, 11946-11951 (1999).
46 Steele-Perkins, G. et al. The transcription factor gene Nfib is essential for both lung maturation and brain development. Molecular and cellular biology 25, 685-698, doi:10.1128/MCB.25.2.685-698.2005 (2005).
47 Denny, S. K. et al. Nfib Promotes Metastasis through a Widespread Increase in Chromatin Accessibility. Cell 166, 328-342, doi:10.1016/j.cell.2016.05.052 (2016).
48 Pauklin, S. & Vallier, L. The cell-cycle state of stem cells determines cell fate propensity. Cell 155, 135-147, doi:10.1016/j.cell.2013.08.031 (2013).
49 Chambers, S. M. et al. Highly efficient neural conversion of human ES and iPS cells by dual inhibition of SMAD signaling. Nat Biotechnol 27, 275-280, doi:10.1038/nbt. 1529 (2009).
50 Maroof, A. M. et al. Directed differentiation and functional maturation of cortical interneurons from human embryonic stem cells. Cell Stem Cell 12, 559-572, doi:10.1016/j.stem.2013.04.008 (2013).
51 Anders, S., Pyl, P. T. & Huber, W. HTSeq—a Python framework to work with high-throughput sequencing data. Bioinformatics 31, 166-169, doi:10.1093/bioinformatics/btu638 (2015).
52 Love, M. I., Huber, W. & Anders, S. Moderated estimation of fold change and dispersion for RNA-seq data with DESeq2. Genome Biol 15, 550, doi:10.1186/s13059-014-0550-8 (2014).
53 Langmead, B. & Salzberg, S. L. Fast gapped-read alignment with Bowtie 2. Nat Methods 9, 357-359, doi:10.1038/nmeth.1923 (2012).
54 Ramirez, F. et al. deepTools2: a next generation web server for deep-sequencing data analysis. Nucleic Acids Res 44, W160-165, doi:10.1093/nar/gkw257 (2016).
55 Heinz, S. et al. Simple combinations of lineage-determining transcription factors prime cis-regulatory elements required for macrophage and B cell identities. Mol Cell 38, 576-589, doi:10.1016/j.molcel.2010.05.004 (2010).
56 Robinson, J. T. et al. Integrative genomics viewer. Nat Biotechnol 29, 24-26, doi:10.1038/nbt.1754 (2011).
57 Steinbeck, J. A. et al. Functional Connectivity under Optogenetic Control Allows Modeling of Human Neuromuscular Disease. Cell Stem Cell 18, 134-143, doi:10.1016/j.stem.2015.10.002 (2016).
58 Chiricozzi, E. et al. Group IIA secretory phospholipase A2 (GIIA) mediates apoptotic death during NMDA receptor activation in rat primary cortical neurons. J Neurochem 112, 1574-1583, doi:10.1111/j.1471-4159.2010.06567.x (2010).
59 Cheng, P. Y. et al. Interplay between SIN3A and STAT3 mediates chromatin conformational changes and GFAP expression during cellular differentiation. PLoS One 6, e22018, doi:10.1371/journal.pone.0022018 (2011).

Although the presently disclosed subject matter and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, and composition of matter, means, methods and steps described in the specification. As one of ordinary skill in the art will readily appreciate from the disclosure of the presently disclosed subject matter, processes, machines, manufacture, compositions of matter, means, methods, or steps, presently existing or later to be developed that perform substantially the same function or achieve substantially the same result as the corresponding embodiments described herein may be utilized according to the presently disclosed subject matter. Accordingly, the appended claims are intended to include within their scope such processes, machines, manufacture, compositions of matter, means, methods, or steps.

Patents, patent applications, publications, product descriptions and protocols are cited throughout this application the disclosures of which are incorporated herein by reference in their entireties for all purposes.

What is claimed is:

1. An in vitro method for differentiating pluripotent stem cells, comprising promoting NFIA signaling in a population of cells expressing one or more neural stem cell marker to obtain a cell population comprising at least about 10% differentiated cells expressing one or more glial competent cell marker.

2. The method of claim 1, wherein the cells expressing one or more neural stem cell marker are obtained by a method comprising exposing a population of stem cells to an effective amount of one or more inhibitor of SMAD signaling.

3. The method of claim 2, wherein the initial promotion of NFIA signaling is at least about 8 days from the initial exposure of the stem cells to the one or more inhibitor of SMAD signaling.

4. The method of claim 1, wherein a detectable level of the one or more glial competent cell marker is present at least about 5 days from the initial promotion of NFIA signaling in the cells.

5. The method of claim 1, wherein the level of expression of functional NFIA activity is decreased in the plurality of cells after the presence of a detectable level of the one or more glial competent cell marker.

6. The method of claim 5, wherein the level of expression of functional NFIA activity is decreased by at least about 90%.

7. The method of claim 1, wherein the differentiated cells do not express a detectable level of one or more neuronal marker.

8. The method of claim 7, wherein the one or more neuronal marker is selected from the group consisting of Tuj1, MAP2, and DCX.

9. The method of claim 1, wherein said promoting NFIA signaling comprises (a) exposing the cells to one or more activator of NFIA or (b) increasing expression of NFIA.

10. The method of claim 9, wherein the one or more activator of NFIA comprises (a) NFIA protein exogenously exposed to the stem cells, (b) a recombinant NFIA protein expressed by the stem cells, or (c) an upstream activator of NFIA.

11. The method of claim 10, wherein the upstream activator of NFIA is TGFβ1.

12. The method of claim 9, wherein said increasing expression of NFIA comprises modifying the NSCs to induce overexpression of NFIA.

13. The method of claim 12, the overexpressed NFIA is a recombinant NFIA protein that is expressed by a NFIA nucleic acid.

14. The method of claim 13, wherein the NFIA nucleic acid is operably linked to an inducible promoter.

15. An in vitro method for differentiating stem cells, comprising exposing a population of stem cells to one or more inhibitor of SMAD signaling, and exposing the cells to fetal bovine serum, to obtain a cell population comprising at least about 10% differentiated cells expressing one or more astrocyte marker.

16. The method of claim 15, wherein the stem cells are differentiated to said cell population at least about 30 days from the initial exposure of the cells to the fetal bovine serum.

17. An in vitro method for differentiating pluripotent stem cells, comprising lengthening G1 phase of the cell cycle of a population of cells expressing one or more neural stem cell marker to obtain a cell population comprising at least about 10% differentiated cells expressing one or more glial competent cell marker.

18. The method of claim 1, wherein the neural stem cell marker is selected from the group consisting of (a) PAX6, NESTIN, SOX1, SOX2, PLZF, ZO-1, and BRN2, or (b) PAX6, SOX1, PLZF, and ZO-1.

19. An in vitro method for differentiating stem cells, comprising exposing a population of stem cells to one or more inhibitor of SMAD signaling, and lengthening G1 phase of the cell cycle of the cells, to obtain a cell population comprising at least about 10% differentiated cells expressing one or more glial competent cell marker.

20. The method of claim 19, wherein the initial lengthening of the G1 phase is at least about 8 days from the initial exposure of the cells to the one or more inhibitor of SMAD signaling.

21. The method of claim 19, wherein a detectable level of the one or more glial competent cell marker is present at least about 10 days from the initial lengthening of the G1 phase.

22. The method of claim 19, wherein said lengthening the G1 phase comprises (a) exposing the cells to one or more G1 phase lengthening compound, or (b) increasing expression of FZR1.

23. The method of claim 22, wherein the one or more G1 lengthening compound comprises a small molecule compound.

24. The method of claim 23, wherein the small molecule compound comprises Olomoucine (Olo).

25. The method of claim 1, wherein the one or more glial competent cell marker is selected from the group consisting of CD44, AQP4, SOX2 and NESTIN.

26. The method of claim 1, wherein the cells expressing one or more glial competent cell marker are cortical glial competent cells or spinal glial competent cells.

27. The method of claim 1, further comprising subjecting the cell population comprising at least about 10% cells expressing one or more glial competent cell marker to conditions suitable to promote differentiation of the cells into cells expressing one or more astrocyte marker.

28. The method of claim 27, wherein the conditions comprise exposing the cell population to leukemia inhibitory factor (LIF), one or more derivative thereof, one or more analog thereof, and/or one or more activator thereof.

29. The method of claim 28, wherein the initial exposure of the cells to LIF, one or more derivative thereof, one or more analog thereof, and/or one or more activator thereof is at least about 10 days from the initial exposure of the stem cells to the one or more inhibitor of SMAD signaling.

30. The method of claim 27, wherein the one or more astrocyte marker is selected from the group consisting of GFAP, AQP4, CD44, S100b, SOX9, NFIA, GLT-1, and CSRP1.

31. The method of claim 30, wherein the one or more astrocyte marker comprises GFAP.

32. The method of claim 27, wherein cells expressing one or more astrocyte marker are cortical astrocytes or spinal cord astrocytes.

33. The method of claim 2, wherein the one or more inhibitor of SMAD signaling comprises one or more inhibitor of transforming growth factor beta (TGFβ)/Activin-Nodal signaling, and one or more inhibitor of bone morphogenetic protein (BMP) signaling.

34. The method of claim 33, wherein the one or more inhibitor of TGFβ/Activin-Nodal signaling comprises a compound selected from the group consisting of SB431542, derivatives thereof, and mixtures thereof.

35. The method of claim 33, wherein the one or more inhibitor of bone morphogenetic protein (BMP) signaling comprises a compound selected from the group consisting of LDN193189, derivatives thereof, and mixtures thereof.

36. An in vitro method for differentiating stem cells, comprising exposing a population of stem cells to an effective amount of one or more inhibitor of SMAD signaling, and exposing the cells to an effective amount of one or more activator of retinoic acid (RA) signaling (“RA activator”) and an effective amount of one or more activator of Sonic hedgehog (SHH) signaling (“SHH activator”), to obtain a cell population comprising at least about 10% differentiated cells expressing one or more spinal cord progenitor marker.

37. The method of claim 36, wherein the population of stem cells are initially exposed to one or more RA activator and one or more SHH activator at least one day from the initial exposure of the cells to the one or more inhibitor of SMAD signaling.

38. The method of claim 36, wherein a detectable level of the one or more spinal cord progenitor marker is present at least about 12 days from the initial exposure of the cells to the one or more RA activator and one or more SHH activator.

39. The method of claim 36, wherein the one or more spinal cord progenitor marker is selected from the group consisting of HOXB4, ISL1, and NKX6.1.

40. The method of claim 1, wherein the stem cells are human stem cells.

41. The method of claim 1, wherein the stem cells are pluripotent stem cells.

42. The method of claim 41, wherein the pluripotent stem cells are selected from the group consisting of embryonic stem cells, induced pluripotent stem cells, and combinations thereof.

43. The method of claim 1, wherein the stem cells are selected from the group consisting of embryonic stem cells, induced pluripotent stem cells, human parthenogenetic stem cells, primordial germ cell-like pluripotent stem cells, epiblast stem cells, F-class pluripotent stem cells, and combinations thereof.

44. A population of in vitro differentiated cells expressing at least about 10% one or more glial competent cell marker and/or one or more astrocyte marker, wherein said differentiated cell population is derived from the method of claim 1.

45. A composition comprising the population of claim 44.

46. A method of treating a neurodegenerative disorder, or reducing damage from a neurological injury, in a subject, comprising administering an effective amount of the population of claim 44 to a subject in need thereof.

47. A kit for inducing differentiation of stem cells, comprising one or more of:
(a) one or more inhibitor of transforming growth factor beta (TGFβ)/Activin-Nodal signaling,
(b) one or more inhibitor of BMP signaling;
(c) one or more activator of NFIA;
(d) LIF, one or more derivative thereof, one or more analog thereof, and/or a one or more activator thereof; and
(e) instructions for inducing differentiation of the stem cells into a cell population comprising at least about 10% differentiated cells expressing one or more glial competent cell marker and/or one or more astrocyte marker.

48. A composition comprising a population of in vitro differentiated cells, wherein at least about 50% of the population of cells express one or more NSC marker and wherein less than about 25% of the population of cells express one or more stem cell marker.

49. A composition comprising a population of in vitro differentiated cells, wherein at least about 50% of the population of cells express one or more glial competent NSC marker or glial competent cell marker, and wherein less than about 25% of the population of cells express one or more marker selected from the group consisting of stem cell markers, NSC markers, and neuronal markers.

50. A composition comprising a population of in vitro differentiated cells, wherein at least about 50% of the population of cells express one or more astrocyte marker, and wherein less than about 25% of the population of cells express one or more marker selected from the group consisting of stem cell markers, NSC markers, neuronal markers, and glial competent cell markers/glial competent NSC markers.
